(19) United States
(12) Patent Application Publication
(10) Pub. No.: US 2003/0165495 A1
(43) Pub. Date: Sep. 4, 2003

(54) Nucleic acids and polypeptides

(76) Inventors: John P. Carulli, Southborough, MA (US); Alexander V. Lukashin, Charlestown, MA (US); Daniel R. Kilburn, Lexington, MA (US); Punam Mathur, Cambridge, MA (US)

Correspondence Address: FISH & NEAVE, 1251 AVENUE OF THE AMERICAS, 50TH FLOOR, NEW YORK, NY, 10020-1105, US

(21) Appl. No.: 10/312,495

(22) PCT Filed: Jun. 22, 2001
(86) PCT No.: PCT/US01/20038

(51) Int. Cl. A61K 39/395; C07H 21/04; C12N 5/06; C12P 21/02; C07K 16/42; C07K 16/00

(52) U.S. Cl. 424/130.1; 530/387.1; 435/69.1; 435/320.1; 435/326; 530/387.2; 536/23.53

(57) ABSTRACT

An isolated polynucleotide encoding a novel immunoglobulin superfamily member named GP286 is provided. GP286 has a predicted single membrane spanning domain and one immunoglobulin (Ig) domain in the extracellular portion of the protein. The protein structure and tissue distribution of GP286 indicate that it plays a role in cell-cell recognition, binding, signaling and adhesion events in the immune system. Provided by the invention are isolated GP286 related polynucleotides and polypeptides, vectors, and host cells comprising any of the above, antibodies directed to GP286, cells which produce such antibodies, and related diagnostic and therapeutic methods.

### RELATED APPLICATIONS

The present application claims priority from U.S. Provisional Application No. 60/283,813, filed Apr. 13, 2001, and from U.S. Provisional Application No. 60/213,630, filed Jun. 23, 2000, each of the disclosures of which is incorporated herein by reference.

### FIELD OF THE INVENTION

The present invention relates generally to the field of molecular biology. More particularly, this invention relates to members of the immunoglobulin superfamily.

### BACKGROUND OF THE INVENTION

Mechanisms through which the cells of an organism communicate with each other and obtain information and stimuli from their environment include cell membrane receptor molecules expressed on the cell surface. Cell surface receptors are a first essential link for translating an extracellular signal into a cellular physiological response. Many such receptors have been identified, characterized, and sometimes classified into major receptor superfamilies based on conserved structural motifs and biological functions such as signal transduction features. A superfamily is broadly defined as a group of proteins that share a certain degree of sequence homology, usually at least 15%. The conserved sequences shared by superfamily members often contribute to the formation of compact tertiary structures referred to as domains, and often the entire sequence of a domain characteristic of a particular superfamily is encoded by a single exon (see, e.g., Abbas et al., CELLULAR AND MOLECULAR IMMUNOLOGY, W. B. Saunders Co., Philadelphia, Pa. 1997). Members of a superfamily are likely derived from a common precursor gene by divergent evolution, and multidomain proteins may belong to more than one superfamily. Examples of protein superfamiies include the ligand-gated ion channel receptor superfamily, the voltage-dependent ion channel receptor superfamily, the receptor tyrosine kinase superfamily, the receptor protein tyrosine phosphatase superfamily, the G protein-coupled receptor superfamily, and the immunoglobulin (Ig) superfamily.

The immunoglobulin (Ig) superfamily encompasses a number of structurally related proteins with a wide variety of functions. The Ig superfamily molecules mediate recognition, adhesion and binding in the immune system (see, e.g., Abbas et al., supra), as well as other functions outside of the immune system. Originally named for immunoglobulin heavy and light chains, members of the Ig superfamily share partial amino acid sequence homology and structural features with Ig, such as the so-called “Ig domains” Ig domains are three-dimensional globular structures having about 70 to 110 amino acid residues and an internal Cys-Cys disulfide bond. These domains contain two layers of P-pleated sheet, each layer composed of three to five antiparallel strands of five to ten amino acid residues. Ig domains are classified as V-like or C-like on the basis of closest homology to either the Ig V or C domains. For a general review, see, e.g., Abbas et al., supra.

Most identified members of the Ig superfamily are integral plasma membrane proteins with one or more Ig domains in the extracellular portions and widely divergent cytoplasmic tails, usually with no intrinsic enzymatic activity. There are exceptions to these generalizations. One recurrent characteristic of the Ig superfamily members is that interactions between Ig domains on different polypeptide chains (of the same or different amino acid sequences) are essential for the biological activities of the molecules. Heterophilic interactions can also occur between Ig domains on entirely distinct molecules expressed on the surfaces of different cells. Such interactions provide adhesive forces that stabilize cell-cell binding.

Many members of the Ig superfamily are cell surface or soluble molecules that mediate cell recognition, adhesion and binding functions in the vertebrate immune system. Two prominent cell types that produce Ig superfamily molecules are B and T lymphocytes. Exemplary Ig superfamily member proteins of importance in the immune system include antibodies, T cell receptors, Class I and II major histo-compatibility complex (MHC) molecules, CD2, CD3, CD4, CD5, CD8, CD28, CD20 (B1), CD32 (FcgRII), CD44, CD54 (ICAM-1), CD80 (B7-1), CD86 (B7-2), CD90 (Thy-1), CD102 (ICAM-2), CD106 (VCAM-1), CD121 (IL-1R), CD152 (CTLA-4), p-IgR, NCAM, and CD140 (PDGFR) (Abbas et al., supra).

B lymphocytes are responsible for humoral immunity. Within B cells, a genetic recombination of genes encoding immunoglobulin heavy chains and immunoglobulin light chains occurs. Ig heavy and light chains are composed of regions derived from separate genes. Both heavy and light chains have variable (V), constant (C), and joining (J) segments which are joined in-frame by genetic recombination during B cell maturation (Abbas, et al. 1997). In addition heavy chains have an additional segment called the diversity (D) segment (Abbas, et al. 1997). The genetic rearrangement allows the expression of functional molecules called Ig or antibodies. Due to a large number of genes that may be used to create the heavy and light chains during genetic recombination, a plethora of antibodies may be produced that have specificity for a vast array of antigens.

B cells produce Ig in both membrane-bound forms and secreted forms. A B cell is immunologically activated when antigen binds to a membrane-bound antibody on the surface of the B cell, and under conditions wherein necessary soluble factors are present that “help” B cells produce antibodies in secreted form.

T lymphocytes are responsible for cell-mediated immunity. A T cell is immunologically activated when it interacts with antigen presenting cells through the T cell receptor, as well as through a number of co-receptors and adhesion molecules. The T cell receptor is a multiprotein complex that consists of variable αβ or γδ receptors associated with invariant CD3 γ, δ, ε, and ζ proteins. T cell receptors specifically recognize MHC-antibody complexes on antigen presenting cells (Cantrell et al, 1995. pp. 151-163 in T Cell Receptors, Bell, J. I., M. J. Owen and E. Simpson, Eds. Oxford University Press, New York). Other T cell surface molecules contributing to MHC recognition include CD4 and CD8, while molecules such as CD28, CTLA4, CD5 and CD44 recognize molecules other than MHC on the surface of antigen producing cells.

Ig superfamily members continue to be identified in immune and non-immune tissues, for instance, in the nervous system. Based on their conserved structural motifs, these novel Ig superfamily members are thought to perform cell recognition, binding and adhesion functions as well. Novel Ig superfamily members will be suitable targets for immune, auto-immune and immunological disease therapies because Ig superfamily members act in conjunction with other Ig superfamily members in the immune system and in non-immune systems.

### SUMMARY OF THE INVENTION

The present invention is based, at least in part, on the discovery of a gene encoding a heretofore unknown Ig superfamily member, termed GP286. (Unless indicated otherwise, the name in lower case, gp286, refers to the new nucleic acids of the invention, whereas the name in uppercase, GP286, refers to the new polypeptides of the present invention. Further, the terms “gp286” and “GP286” refer to both splice variants of the invention. See infra.)

The human gp286 cDNA described below (SEQ ID NO: 1) has a 558 base pair open reading frame that encodes an 186 amino acid protein (SEQ ID NO:2). The human gp286 gene is located on chromosome 19 and encodes a GP286 protein predicted to have a single membrane-spanning domain and one immunoglobulin (Ig) domain in the N-terminal, extracellular portion of the protein. gp286 mRNA is enriched in T lymphocytes, and is detectable in various immune system tissues such as peripheral blood leukocytes, lymph nodes, spleen, thymus and tonsil. Furthermore, gp286 mRNA is detectable at low levels in some non-immune tissues, which is likely due to infiltration of T cells into these non-immune tissues. The protein structure and tissue distribution of GP286 indicates a role for GP286 in T cell interactions with other cells or the extracellular environment during an immune response. GP286a appears to be a splice variant of GP286. In contrast to GP286, GP286a has a 834 base pair open reading frame (SEQ ID NO: 14) that encodes a 278 amino acid protein (SEQ ID NO: 15). Like GP286, the GP286a protein is predicted to have a single membrane-spanning domain and one extracellular Ig domain.

Novel Ig superfamily members localized to particular cell types, such as GP286, which is expressed at high levels in T cells, will be useful cell and tissue markers for diagnostic purposes and in forensic science. Tissue-specific Ig superfamily members will also be suitable therapeutic targets for treating abnormal conditions, disorders and/or diseases related to improper cell-cell adhesion and signaling in the tissue, particularly during tissue development or during tissue regeneration, e.g., after tissue damage or trauma. For instance, GP286 and GP286a are useful in mediating cell adhesion of T cells to extracellular matrix molecules and to other cells.

The invention features a polynucleotide which includes a nucleotide sequence that encodes a protein which comprises an amino acid sequence that is at least 80% (85%, 95% or 98%) identical to the amino acid sequence of SEQ ID NO:2, or to the Ig domain of SEQ ID NO:2. In some embodiments, a polynucleotide has the sequence of SEQ ID NO:1, or a fragment thereof having at least 17 nucleic acid units (e.g., nucleotides). Examples of such fragments are SEQ ID NOs:3, 4, 5, 16, 20, 22, 26, 28 and 32.

In some embodiments, the invention features a polynucleotide which includes a nucleotide sequence that encodes a protein which comprises an amino acid sequence that is at least 80% (85%, 95% or 98%) identical to the amino acid sequence of SEQ ID NO:15 or to the Ig domain of SEQ ID NO:15. In some embodiments, a polynucleotide has the sequence of SEQ ID NO: 14, or a fragment thereof having at least 17 nucleic acid units (e.g., nucleotides). Examples of such fragments are SEQ ID NOs:18, 24 and 30.

In some embodiments, a polynucleotide comprises the sequence of SEQ ID NO: 13, or a fragment thereof having at least 17 nucleic acid units. Examples of such fragments are SEQ ID NOs: 3, 4, 5, 16, 18, 20, 22, 24, 26, 28, 30 and 32.

The invention provides a polynucleotide which encodes a naturally occurring allelic variant of a polypeptide comprising the amino acid sequence of any one of SEQ ID NOs:2 or 15, wherein the nucleic acid hybridizes to any one of SEQ ID NOs:1, 13 or 14 under stringent conditions.

The invention also provides isolated polynucleotides that encode select portions of GP286. As will be further discussed herein below, these “nucleic acid fragments” can be used, for example, to express specific portions of the GP286, either alone or as elements of a fusion protein. A nucleic acid fragment may also be used as a region-specific nucleic probe.

Also provided by the invention is an isolated GP286 protein comprising an amino acid sequence that is at least 80% (85%, 95% or 98%) identical to an Ig domain encoded by the amino acid sequence of either of SEQ ID NOs:2 or 15.

The invention also provides an isolated GP286 protein encoded by a polynucleotide having a sequence which is at least about 65%, preferably 75%, 85%, or 95% identical to either of SEQ ID NOs:1 or 14, and an isolated protein encoded by a polynucleotide having a sequence which hybridizes under stringent conditions to a nucleic acid having the sequence of either of SEQ ID NOs:1 or 14.

The invention provides gp286 polynucleotides that specifically detect gp286 nucleic acids relative to nucleic acids encoding other members of the Ig superfamily. The invention also provides a nucleic acid construct, e.g., a recombinant vector such as an expression vector, comprising gp286 polynucleotides of the invention.

The present invention also provides a diagnostic assay for identifying the presence or absence of a genetic lesion or mutation characterized by at least one of: (i) aberrant modification or mutation of a gene encoding a GP286 protein; (ii) mis-regulation of a gene encoding a GP286 protein; and (iii) aberrant post-translational modification of a GP286 protein, wherein a wild-type form of the gene encodes a protein with a GP286 biological activity.

Host cells containing such nucleic acid constructs are also provided, as is a method for producing a GP286 polypeptide by culturing, in a suitable medium, a host cell of the invention containing a recombinant expression construct such that a GP286 polypeptide is produced.

Isolated or recombinant GP286 proteins and polypeptides are provided by the invention. Preferred GP286 proteins and polypeptides possess at least one of the following (overlapping) biological activities possessed by naturally occurring human GP286: (1) the ability to interact with (e.g., bind to) a ligand (e.g., a protein receptor, a polysaccharide, etc.) that naturally binds to GP286 protein; (2) the ability to bind to an auto-antibody to naturally occuring human GP286 or an antibody raised against naturally occurring human GP286; (3) the ability to participate in a T lymphocyte-mediated function (e.g., a signal transduction function in immune development); and (4) the ability to mediate cell-cell interactions such as recognition, binding and/or adhesion.

The GP286 proteins or biologically active portions thereof can be operably linked to a non-GP286 polypeptide (e.g., heterologous amino acid sequences, such as sequences that facilitate protein stability, detection, purification, or in vivo delivery to target cells) to form GP286 fusion proteins. The invention further features antibodies (e.g., polyclonal or monoclonal antibodies), including humanized and fully human antibodies, that specifically bind to GP286 proteins or portions thereof.

The above-described gp286-related isolated polynucleotides, GP286 proteins or biologically active portions thereof, antibodies or fusion proteins, can be incorporated into pharmaceutical compositions, which optionally include pharmaceutically acceptable carriers.

Such compositions are useful in therapeutic methods for ameliorating disorders, conditions and diseases in a subject associated with abnormal GP286 cellular localization, expression and/or activity. The present invention thus provides methods of treatment comprising the step of administering a gp286-related compound or composition of the invention. Such methods will be useful, for example, for treating abnormal conditions affecting T cells, and disorders or diseases which correlate with cell recognition, binding, signaling and adhesion functions in the developing or adult immune and/or central nervous system.

The invention provides a method for modulating GP286 activity. In this method, a target cell is contacted with an agent that modulates (e.g., inhibits or stimulates) GP286 activity or expression such that the GP286 activity or expression is altered. In some embodiments, the agent is an antibody that specifically binds to GP286. In some embodiments, the agent modulates the GP286 activity or expression by modulating transcription of a gp286 gene, splicing of gp286 RNA, or translation of a gp286 mRNA. In other embodiments, the agent is a nucleic acid having a sequence that is antisense to the coding strand of the gp286 mRNA or the gp286 gene. In other embodiments, the agent can be a GP286 protein, a nucleic acid encoding a GP286 protein, or an antagonist or agonist of the GP286 protein such as a peptide, a peptidomimetic, or other small molecules.

The present invention provides a method for detecting the presence of a gp286 polynucleotide, a GP286 protein or its activity in a biological sample (e.g., a fluid or tissue sample derived from a patient) by contacting the sample with an agent capable of detecting an indicator of the presence of gp286 polynucleotide sequences, GP286 protein or its activity.

The invention also provides a method for identifying a compound that binds to a GP286 protein. In another aspect, the invention provides a method for identifying a compound that modulates the biological activity of a GP286 protein, comprising measuring a biological activity or expression of the protein in the presence and absence of a test compound and identifying those compounds which alter the activity of the protein. Combinatorial libraries can be used as sources of candidate compounds in these methods.

The invention provides a non-human animal (e.g., a mammal such as a mouse, rat, guinea pig, sheep, goat, horse or cow) at least some cells of which comprise a polynucleotide of this invention. Such an animal can be chimeric where only some of its somatic and/or germ cells carry the polynucleotide. Such an animal can alternatively be transgenic where all of its somatic and germ cells carry the polynucleotide.

The invention also provides a non-human animal whose endogenous ortholog of the gp286 gene is disrupted by gene targeting (i.e., “knocked out”). Cells containing a gp286 polynucleotide, biological samples such as tissues and fluids and GP286-related products derived from these and the above-mentioned animals are also within the scope of this invention.

The invention provides a computer readable means of storing the nucleic acid and amino acid sequences of the instant invention. The records of the computer readable means can be accessed for reading and display of sequences and for comparison, alignment and ordering of the sequences of the invention to other sequences.

Other features and advantages of the invention will be apparent from the following detailed description, drawings, and from the claims.

### BRIEF DESCRIPTION OF THE DRAWINGS

FIGS. 1A-1B The nucleotide and deduced amino acid sequences of GP286. The immunoglobulin (Ig) domain in the extracellular portion is underlined and the transmembrane domain is boxed. The cDNA is 973 bases long. See SEQ ID NOs:1 and 2.

FIGS. 2A-2B The nucleotide and deduced amino acid sequences of GP286a. The immunoglobulin (Ig) domain in the extracellular portion is underlined and the transmembrane domain is boxed. The exon-intron boundaries are denoted by a backslash (/). The cDNA is 1065 bases long. See SEQ ID NOs: 14 and 15.

FIG. 3 Expression of GP286 in human immune system tissues as determined by reverse transcription polymerase chain reaction (RT-PCR). RT-PCR was performed as described in the text. BM=bone marrow, PBL=peripheral blood lymphocytes, Li=liver, LN=lymph nodes, Sp=spleen, Thy=thymus, -=no template control, Genomic=genomic DNA control lane.

FIG. 4 Expression of GP286 RNA in human tissues as determined by RT-PCR. B=brain, H=heart, K=kidney, Li=liver, Lg=lung, Pan=pancreas, Pl=placenta, SkM=skeletal muscle, NTC=no template control, LN=lymph nodes, II-23=Mouse T Cell Line II-23, G=genomic DNA control lane.

FIG. 5 Expression of GP286 RNA in human cell lines by Northern blotting. Lane 1: G-361 (melanoma); Lane 2: A549 (lung carcinoma); Lane 3: SW480 (colorectal adenocarcinoma); Lane 4: Raji (Burkitt's lymphoma); Lane 5: MOLT-4 (lymphoblastic leukemia); Lane 6: K-562 (chronic myelogenous leukemia); Lane 7: HeLa (cervical adenocarcinoma); and Lane 8: HL-60 (promyelocytic leukemia).

FIGS. 6A-6H The nucleotide sequence of human genomic gp286, with gp286 exons underlined. The methionine codon is double-underlined and the stop codon is thick-underlined. See SEQ ID NO:13.

FIGS. 7A-7H The nucleotide sequence of human genomic gp286, with gp286a exons highlighted and underlined. The initiating methionine codon is double-underlined and the stop codon is thick-underlined. See SEQ ID NO:13.

FIG. 8 Homology between GP286 (SEQ ID NO:2, top sequence) and mouse T cell receptor beta chain (SEQ ID NO:6, bottom sequence). Vertical lines show identity and dots indicate gaps.

### DETAILED DESCRIPTION OF THE INVENTION

The present invention is based, at least in part, on the discovery of a novel human gene encoding a heretofore unknown protein, GP286. This gene, gp286, was identified by computational analysis of (“mining”) the published nucleic acid sequences of the human genome. The gp286 gene is alternatively spliced and normally resides on human chromosome 19. One transcript, gp286, contains 4 exons, while gp286a contains 5 exons. Exons 2 and 3 are the same in both the gp286 and gp286a transcripts and exon 1 is similar; however, gp286 comprises exon 4 while gp286a comprises exons 4a (which contains part of exon 4) and exon 5. The gp286 mRNA transcribed from this gene is approximately 971 bases in length, has an open reading frame of 558 base pairs, and encodes a protein predicted to be 186 amino acid residues. The gp286a mRNA transcribed from this gene is approximately 1065 bases in length, has an open reading frame of 834 base pairs, and encodes a protein predicted to be 278 amino acid residues. The novel GP286 proteins are predominantly expressed in the immune system.

Definitions

As used herein, “nucleic acid” (also “polynucleotide”) includes DNA molecules (e.g., cDNA or genomic or synthetic DNA) and RNA molecules (e.g., mRNA). The term also is intended to include analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. See, e.g., Baner et al, Curr. Opin. Biotechnol. 12:11-15 (2001); Escude et al., Proc. Natl. Acad. Sci. USA 14;96(19):10603-7 (1999); Nilsson et al., Science 265(5181):2085-8 (1994); Praseuth et al., Biochim. Biophys. Acta. 1489(1):181-206 (1999); Fox, Curr. Med Chem. 7(1):17-37 (2000); Kochetkova et al., Methods Mol. Biol. 130:189-201 (2000); Chan et al., J. Mol. Med 75(4):267-82 (1997).

As used herein, an “isolated nucleic acid” (also “isolated polynucleotide”) is one which is separated from other nucleic acid molecules that are present in the natural source of the nucleic acid. Specifically excluded are isolated, non-recombinant native chromosomes and fragments thereof that are larger than 500 kilobases. Preferably, an “isolated” nucleic acid is substantially free of sequences that naturally flank that nucleic acid in the genome of the organism from which the nucleic acid is derived. For example, a preferred isolated gp286 nucleic acid is flanked by less than about 10 kb, 5 kb, 4 kb, 3 kb, 2 kb, 1 kb, 0.5 kb or 0.1 kb of nucleotide sequences that naturally flank the nucleic acid in the genomic DNA of the cell from which the isolated nucleic acid is derived.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence (i.e., a sequence that is not naturally adjacent to this endogenous nucleic acid sequence) is placed adjacent to the endogenous nucleic acid sequence, such that the expression of this endogenous nucleic acid sequence is altered. By way of example, a non-native promoter sequence can be substituted (e.g., by homologous recombination) for the native promoter of a gp286 gene in the genome of a human cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous gp286-coding sequence is considered “isolated” if it contains an insertion, deletion or a point mutation introduced artificially, e.g., by human intervention.

An “isolated nucleic acid” also includes a nucleic acid integrated into a host cell chromosome at a heterologous site, a nucleic acid construct present as an episome and a nucleic acid construct integrated into a host cell chromosome.

Moreover, an “isolated nucleic acid” can be substantially free of other cellular material, or substantially free of culture medium when produced by recombinant techniques, or substantially free of chemical precursors or other chemicals when chemically synthesized.

A polynucleotide of the invention is considered “full-length” if it is able to encode a full-length GP286 protein.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence.

As used herein, the term “microarray” (also “nucleic acid microarray”) refers to a substrate-bound plurality of nucleic acids, hybridization to each of the bound nucleic acids being separately detectable. The substrate can be solid or porous, planar or non-planar, unitary or distributed, or in any other configuration.

As so defined, the term “microarray” includes all the devices so called or similarly called in Schena (ed.), DNA Microarrays: A Practical Approach (Practical Approach Series), Oxford University Press (1999) (ISBN: 0199637768); Nature Genet. 21(1)(suppl): 1-60 (1999); and Schena (ed.), Microarray Biochip: Tools and Technology, Eaton Publishing Company/BioTechniques Books Division (2000) (ISBN: 1881299376); Brenner et al., Proc. Natl. Acad. Sci. USA 97(4):1665-1670 (2000). The disclosures of all of these references are incorporated herein by reference in their entireties.

As used herein with respect to nucleic acid hybridization, the term “probe” (also “nucleic acid probe” or “hybridization probe”) refers to an isolated nucleic acid of known sequence that is, or is intended to be, detectably labeled. As used herein with respect to a nucleic acid microarray, the term “probe” (or equivalently “nucleic acid probe” or “hybridization probe”) refers to the isolated nucleic acid that is, or is intended to be, bound to the substrate. In either such context, the term “target” refers to a nucleic acid intended to be bound to a probe by sequence complementarity.

Unless otherwise indicated, a “nucleic acid comprising SEQ ID NO:X” refers to a nucleic acid, at least a portion of which has either (i) the sequence of SEQ ID NO:X, or (ii) a sequence complementary to SEQ ID NO:X The choice between the two is dictated by the context. For instance, if the nucleic acid is used as a probe, the choice between the two is dictated by the requirement that the probe be complementary to the desired target.

For purposes herein, “high stringency conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated that hybridization at 65° C. will occur at different rates depending on a number of factors (discussed infra), including the length and percent identity of the sequences which are hybridizing.

For microarray-based hybridization, standard “high stringency conditions” are defined as hybridization in 50% formamide, 5×SSC, 0.2 μg/μl poly(dA), 0.2 μg/μl human cotl DNA, and 0.5% SDS, in a humid oven at 42° C. overnight, followed by successive washes of the microarray in 1×SSC, 0.2% SDS at 55° C. for 5 minutes, and then 0.1×SSC, 0.2% SDS, at 55° C. for 20 minutes. For microarray-based hybridization, “moderate stringency conditions”, suitable for cross-hybridization to mRNA encoding structurally- and functionally-related proteins, are defined to be the same as those for high stringency conditions but with reduction in temperature for hybridization and washing to room temperature (approximately 25° C.).

As used herein, the terms “protein,” “polypeptide,” and “peptide” are used interchangeably to refer to a naturally-occurring or synthetic polymer of amino acids, irrespective of length, where amino acids here include naturally-occurring amino acids, naturally-occurring amino acid structural variants, and synthetic non-naturally occurring analogs that are capable of participating in peptide bonds. The terms “protein”, “polypeptide”, and “peptide” explicitly permit post-translational and post-synthetic modifications, such as N- or C-terminal amino acid cleavage reactions and glycosylation.

The term “oligopeptide” herein denotes a protein, polypeptide, or peptide having 25 or fewer amino acid residues.

A protein, polypeptide, peptide or oligopeptide is considered “isolated” when it is encoded by an isolated polynucleotide; when it exists in a purity not found in nature, where purity can be adjudged with respect to the presence of other cellular material; and/or when it includes amino acid analogs or derivatives not found in nature or linkages other than standard peptide bonds. As thus defined, “isolated” does not necessarily require that the protein, polypeptide, peptide or oligopeptide so described has been physically removed from its native environment.

A protein, polypeptide, peptide or oligopeptide is considered “purified” herein when it is present at a concentration of at least 65% (e.g., at least 75%, 85% or 95%), as measured on a mass basis with respect to total protein in a composition. It is considered “substantially purified” when the concentration is at least 85%.

As used herein, “orthologs” are separate occurrences of the same gene in different species of organisms. The separate occurrences have similar or identical amino acid sequences, where the degree of sequence similarity depends in part on the evolutionary distance of the species from a common ancestor having the same gene.

As used herein, the term “paralogs” indicates separate occurrences of a gene in one species of organism. The separate occurrences have similar or identical amino acid sequences, where the degree of sequence similarity depends in part on the evolutionary distance of these separate occurrences from the gene duplication event giving rise to the occurrences.

The term “homologs” (also “homologues”) encompasses “orthologs” and “paralogs.”

“Homologous” amino acid sequences include those amino acid sequences which contain conservative amino acid substitutions and which polypeptides have substantially the same binding and/or activity. A homologous amino acid sequence does not, however, include the amino acid sequence encoding other known Ig superfamily members. Homology (percent identity) can be determined by, for example, the GAP program (Wisconsin Sequence Analysis Package, Version 8 for Unix, Genetics Computer Group, University Research Park, Madison Wis.), using the default settings, which uses the algorithm of Smith and Waterman (Adv. Appl. Math., 2:482489 (1981), which is incorporated herein by reference in its entirety).

As used herein, the term “antibody” refers to a full antibody (consisting of two heavy chains and two light chains) or a fragment thereof. Such fragments include, but are not limited to, those produced by digestion with various proteases, those produced by chemical cleavage and/or chemical dissociation, and those produced recombinantly, so long as the fragment remains capable of specific binding to an antigen. Among these fragments are Fab, Fab′, F(ab′)₂, and single chain Fv (scFv) fragments.

Within the scope of the term “antibody” are also antibodies that have been modified in sequence, but remain capable of specific binding to an antigen. Example of modified antibodies are interspecies chimeric and humanized antibodies; antibody fusions; and heteromeric antibody complexes, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513), the disclosure of which is incorporated herein by reference in its entirety).

“Specific binding” refers to the ability of two molecules to bind to each other in preference to binding to other molecules in the environment.

Typically, “specific binding” discriminates over adventitious binding in a reaction by at least two-fold, more typically by at least 10-fold, often at least 100-fold. Typically, the affinity or avidity of a specific binding reaction is at least about 10₋₇ M (e.g., at least about 10₋₈M or 10₋₉ M).

By the term “region” is meant a physically contiguous portion of the primary structure of a biomolecule. In the case of proteins, a region is defined by a contiguous portion of the amino acid sequence of that protein.

The term “domain” refers to a structure of a biomolecule that contributes to a known or suspected function of the biomolecule. Domains may be co-extensive with regions or portions thereof; domains may also include distinct, non-contiguous regions of a biomolecule. Examples of GP286 protein domains include, but are not limited to, an extracellular domain (i.e., N-terminal), a transmembrane domain, and a cytoplasmic domain (i.e., C-terminal). The extracellular domain can be further subdivided into, e.g., an Ig domain and a signal sequence domain.

As used herein, the term “compound” means any molecule, including, but not limited to, small molecule, peptide, protein, sugar, nucleotide, nucleic acid, lipid, etc., and such a compound can be natural or synthetic.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Standard reference works setting forth the general principles of recombinant DNA technology known to those of skill in the art include Ausubel et al., CURRENT PROTOCOLS IN MOLECULAR BIOLOGY, John Wiley & Sons, New York (1998 and Supplements to 2001); Sambrook et al., Molecular Cloning: A Laboratory Manual, 2d Ed. Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991). Standard reference works setting forth the general principles of immunology known to those of skill in the art include: Harlow and Lane, Antibodies: A Laboratory Manual Cold Spring Harbor Laboratory Press, Cold Spring Harbor, N.Y. (1990); Harlow and Lane, Antibodies: A Laboratory Manual, 2d Ed. Cold Spring Harbor Laboratory Press, Cold Spring Harbor, N.Y. (1999); Roitt et al., IMMUNOLOGY, 3d Ed., Mosby-Year Book Europe Limited, London (1993).

Standard reference works setting forth the general principles of medical physiology and pharmacology known to those of skill in the art include: Harrison's PRINCIPLES OF INTERNAL MEDICINE, 14^{th} Ed., (Anthony S. Fauci et al., editors), McGraw-Hill Companies, Inc., 1998;

GP286 Related Nucleic Acids

In a first series of nucleic acid embodiments, the invention provides isolated polynucleotides that encode the entirety of the GP286 protein. As discussed above, the “full-length” polynucleotides of the present invention can be used, inter alia, to express full length GP286 protein. The full-length polynucleotides can also be used as nucleic acid probes; used as probes, the isolated polynucleotides of these embodiments will hybridize to gp286.

In some embodiments, the invention provides an isolated polynucleotide comprising (i) the nucleotide sequence of SEQ ID NOs: 1, 13 or 14, (ii) a degenerate variant of the nucleotide sequence of SEQ ID NOs: 1, 13 or 14, or (iii) the complement of (i) or (ii). SEQ ID NOs:1 and 14 present an entire cDNA sequence of gp286 and gp286a, respectively, including the 5′ untranslated (UT) region and 3′ UT region. SEQ ID NO: 13 presents the genomic DNA sequence of gp286, including the 5′ and 3′ non-transcribed regions.

In other embodiments, the invention provides an isolated polynucleotide comprising (i) the nucleotide sequence of SEQ ID NOs:3, 4, 5, 16, 18, 20, 22, 24, 26, 28, 30 or 32, (ii) a degenerate variant of the nucleotide sequence of SEQ ID NO:3, or (iii) the complement (i) or (ii).

SEQ ID NO:3 presents an RT-PCR fragment of gp286 amplified from human thymus mRNA using CR2-037 (SEQ ID NO:7) and CR2-038 (SEQ ID NO:8) primers; SEQ ID NO:4 represents a 5′ RACE product resulting from the amplification of human thymus cDNA using the CR2-146 primer (SEQ ID NO:9) and the AP-1 primer (SEQ ID NO:11); SEQ ID NO:5 represents a 3′ RACE product resulting from amplification of human thymus cDNA with the CR2-148 primer (SEQ ID NO: 10) and the AP-1 primer; SEQ ID NO: 16 represents the nucleic acid sequence encoding the extracellular potion of GP286; SEQ ID NO:18 represents the nucleic acid sequence encoding the extracellular portion of GP286a; SEQ ID NO:20 represents the nucleic acid sequence encoding the Ig-like domain of GP286; SEQ ID NO:22 represents the nucleic acid sequence encoding the intracellular domain of GP286; SEQ ID NO:24 represents the nucleic acid sequence encoding the intracellular domain of GP286a; SEQ ID NO:26 represents the nucleic acid sequence encoding the extracellular domain of GP286 without the amino acids N-terminal of the Ig-like domain; SEQ ID NO:28 represents the nucleic acid sequence encoding GP286 not including the portion encoding amino acids N-terminal of the Ig-like domain; SEQ ID NO: 30 represents the nucleic acid sequence encoding GP286a not including the portion encoding the amino acids N-terminal of the Ig-like domain; and SEQ ID NO: 32 represents the nucleic acid sequence encoding the transmembrane domain of GP286.

In some embodiments, the invention provides an isolated polynucleotide comprising (i) a nucleotide sequence that encodes a polypeptide with the amino acid sequence of SEQ ID NOs:2 or 15, or (ii) the complement of a nucleotide sequence that encodes a polypeptide with the amino acid sequence of SEQ ID Nos:2 or 15. SEQ ID NO:2 provides the amino acid sequence of GP286 encoded by the cDNA of SEQ ID NO: 1. SEQ ID NO: 15 provides the amino acid 0.25 sequence of GP286a encoded by the cDNA of SEQ ID NO: 14.

In some embodiments, the invention provides an isolated polynucleotide having a nucleotide sequence that (i) encodes a polypeptide having the sequence of SEQ ID NOs:2 or 15, (ii) encodes a polypeptide having the sequence of SEQ ID NOs:2 or 15 with conservative amino acid substitutions, or (iii) that is the complement of (i) or (ii).

Nucleic Acid Encoding Portions of GP286

In a second series of nucleic acid embodiments, the invention provides isolated polynucleotides that encode select portions of gp286. As will be further discussed herein below, these nucleic acid molecules can be used, inter alia, to express specific portions of the GP286. These nucleic acid molecules can also be used, inter alia, as region-specific nucleic acid probes.

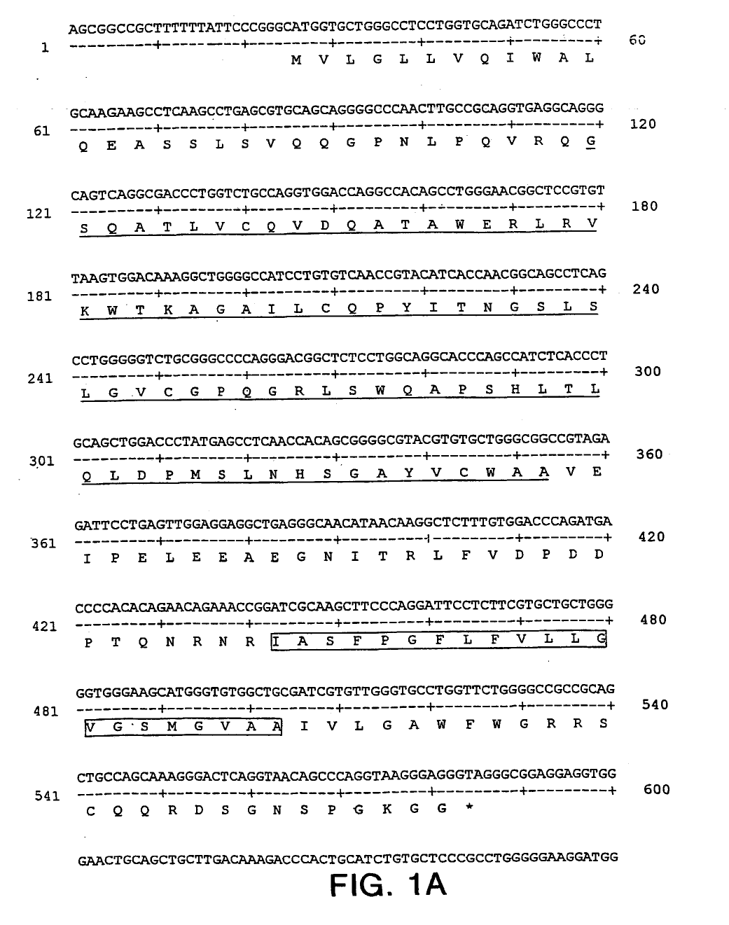

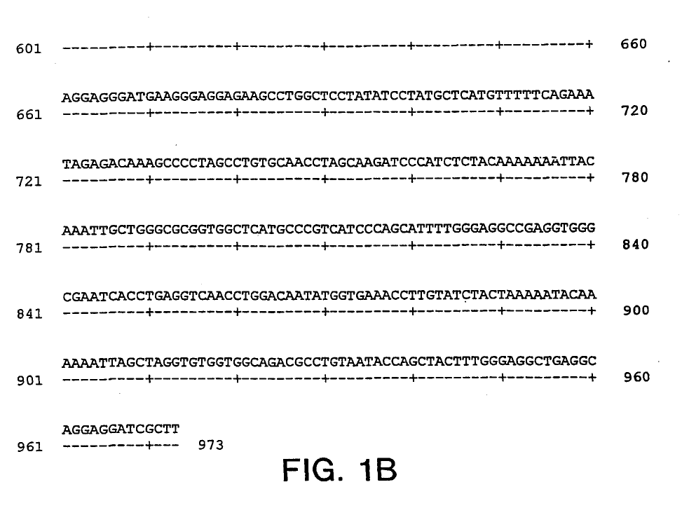

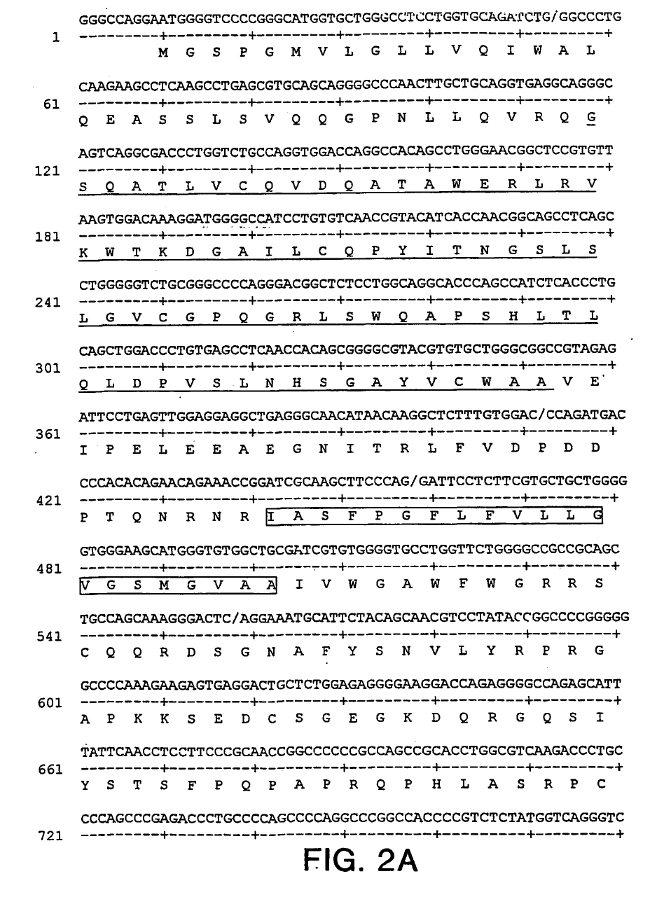

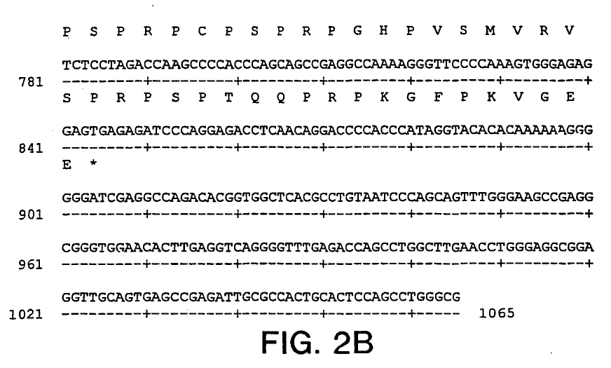

In some embodiments, the isolated polynucleotide encodes, or the complement of which encodes, a polypeptide having, in at least one copy, the Ig domain characteristic of the N-terminal extracellular portion of GP286. Specifically, the extracellular Ig domain (SEQ ID NO:21) is encoded by nucleotides 119-354 of the gp286 cDNA sequence of SEQ ID NO:1 (see FIG. 1) and nucleotides 118-353 of SEQ ID NO: 14 (see FIG. 2).

Preferably, the isolated polynucleotides (or their complements) also encode a signal secretion sequence that will mediate transport of the encoded polypeptides through a membrane. The gp286 signal secretion sequence is encoded by nucleotides 26-76 of the gp286 cDNA sequence of SEQ ID NO:1 (amino acids 1-17 of SEQ ID NO:2; see FIG. 1) and by nucleotides 10-75 of the gp286a cDNA sequence of SEQ ID NO: 14 (amino acids 1-22 of SEQ ID NO: 15; see FIG. 2). More preferably, the signal secretion sequence of the isolated polynucleotide of the invention is from gp286.

The above preferred isolated polynucleotides may optionally encode a transmembrane domain, if insertion of the encoded polypeptides into a membrane is so desired. The transmembrane domain may be encoded by gp286 or gp286a (see below) or may be encoded by a heterologous gene encoding a transmembrane domain of a heterologous membrane-associated protein.

If so desired, the above preferred isolated polynucleotides may further comprise an intracellular C-terminal domain if specific signaling reactions are desired in response to GP286 binding interactions. The intracellular domain may be encoded by gp286 or gp286a (see below), or may be encoded by a heterologous gene encoding an intracellular domain of a heterologous membrane-associated protein.

Other preferred embodiments of the polynucleotides of the invention are those that encode, or the complements of which encode, a polypeptide having the transmembrane domain of GP286. Specifically, the GP286 transmembrane domain is encoded by nucleotides 443-505 of the gp286 cDNA sequence of SEQ ID NO:1 (see FIG. 1) and by nucleotides 442-504 of the gp286a cDNA sequence of SEQ ID NO: 14 (see FIG. 2). SEQ ID NO:33 represents the amino acid sequence of the transmembrane domain of GP286 encoded by SEQ ID NO:32.

Yet other preferred embodiments of the nucleic acids above-described are those that encode, or the complements of which encode, a polypeptide having a (C-terminal) intracellular domain of GP286. Specifically, the intracellular domain of GP286 is encoded by nucleotides 506-583 of the gp286 cDNA sequence of SEQ ID NO: 1 (see FIG. 1). The intracellular domain of GP286a is encoded by nucleotides 505-843 of the gp286a cDNA sequence of SEQ ID NO: 14 (see FIG. 2). SEQ ID NO:23 represents the amino acid sequence of the intracellular domain of GP286 encoded by SEQ ID NO:22, while SEQ ID NO:25 represents the amino acid sequence of the intracellular domain of GP286a encoded by SEQ ID NO:24.

The most significant difference between GP286 and GP286a is in the C-terminal cytoplasmic portion of the protein. Where GP286 has a cytoplasmic tail of about 26 amino acids, GP286a has a cytoplasmic tail of about 113 amino acids. In addition, GP286a has three tyrosine residues in the cytoplasmic tail that are potential sites of protein phosphorylation. This finding strongly suggests that GP286a can be phosphorylated, and thus is likely to play an important role in signal transduction in T cells. GP286 is also likely a receptor, but is unlikely to be phosphorylated. Without being bound to any particular theory, for example, the extracellular portion of the protein, comprising the Ig domain, may receive a signal from an extracellular molecule, and the signal is transmitted through the cytoplasmic domain to elicit a response, e.g., a growth or differentiation response, in T cells.

Preferred isolated polynucleotides of the invention is represented by SEQ ID NO:3. SEQ ID NO: 3 spans part of exons 2 and 3, which exons are identical for both gp286 and gp286a. See Example 3.

Other preferred isolated polynucleotides of the invention include those that distinguish between gp286 and gp286a. One having ordinary skill in the art following the teachings of the specification will recognize that a splice variant-specific polynucleotide may be one that includes only sequences from one splice variant and not the other. Other splice variant-specific polynucleotides include those that can be used to exploit, e.g., a size difference between the different splice variants. Examples of splice variant-specific polynucleotides include those represented by the 3′ 25 nucleotides of SEQ ID NO:22 (gp286) and by the 3′ 286 nucleotides of SEQ ID NO:24 (gp286a), although others are also disclosed herein.

Cross-Hybridizing Nucleic Acids

In another series of nucleic acid embodiments, the invention provides isolated polynucleotides that hybridize to various of the gp286 nucleic acids of the present invention. These “cross-hybridizing nucleic acids” can be used, inter alia, as probes for, and to drive expression of proteins that are related to gp286 of the present invention as further isoforms, homologs, paralogs, or orthologs.

In some embodiments, the invention provides an isolated polynucleotide comprising a sequence that hybridizes under high stringency conditions to a probe the nucleotide sequence of which comprises SEQ ID NO:3, the complement of SEQ ID NO:3, or a fragment thereof having at least 17 nucleic acid units thereof

Preferred Nucleic Acids

Particularly preferred among the above-described nucleic acids are those that are expressed, or the complements of which are expressed, in tissues of the immune system. Also particularly preferred among the above-described nucleic acids are those that encode, or the complements of which encode, a polypeptide having a gp286 biological activity, as described supra.

Nucleic Acid Fragments

In another series of nucleic acid embodiments, the invention provides fragments of various of the isolated polynucleotides of the present invention which prove useful, inter alia, as region-specific nucleic acid probes, as amplification primers, to express specific portions of the GP286, either alone or as elements of a fusion protein and to direct expression or synthesis of epitopic or immunogenic protein fragments.

In some embodiments, the invention provides an isolated polynucleotide comprising at least 17 nucleotides, 18 nucleotides, 20 nucleotides, 24 nucleotides, or 25 nucleotides of contiguous nucleic acid sequence selected from SEQ ID NOs:1 or 14, wherein the isolated polynucleotide is no more than about 100 kb in length, typically no more than about 75 kb in length, more typically no more than about 50 kb in length. Often, the isolated polynucleotides of these embodiments are no more than about 25 kb in length, often no more than about 15 kb in length, and frequently no more than about 10 kb in length. Even more preferably, the isolated polynucleotides are no more than 5000 base pairs, often no more than 1000 base pairs, 500 base pairs, 100 base pairs or 50 base pairs.

In some embodiments, the invention provides an isolated nucleic acid comprising a nucleotide sequence that (i) encodes a polypeptide having the sequence of at least eight contiguous amino acids of either SEQ ID NOs:2 or 15, (ii) encodes a polypeptide having the sequence of at least eight contiguous amino acids of either SEQ ID NOs:2 or 15 with conservative amino acid substitutions, or (iii) is the complement of (i) or (ii), wherein the isolated nucleic acid is no more than about 100 kb in length, typically no more than about 75 kb in length, more typically no more than about 50 kb in length. Often, the isolated nucleic acids of these embodiments are no more than about 25 kb in length, often no more than about 15 kb in length, and frequently no more than about 10 kb in length. Even more preferably, the isolated polynucleotides are no more than 5000 base pairs, often no more than 1000 base pairs, 500 base pairs, 100 base pairs or 50 base pairs.

Single Exon Probes

The invention further provides genome-derived single exon probes having portions of no more than one exon of the gp286 gene. Single exon probes have particular utility in identifying and characterizing splice variants. In particular, such single exon probes are useful for identifying and discriminating the expression of distinct isoforms of gp286.

In some embodiments, the invention provides an isolated nucleic acid comprising a nucleotide sequence selected from one of the following exon-specific portions of SEQ ID NOs: 1, 13 or 14; or the complement of SEQ ID NOs:1, 13 or 14, wherein the portion comprises at least 17 contiguous nucleotides, 18 contiguous nucleotides, 20 contiguous nucleotides, 24 contiguous nucleotides, 25 contiguous nucleotides, or 50 contiguous nucleotides of any one of the portions of SEQ ID NOs:1, 13 or 14, or their complement:

TABLE 1
Exon coordinates of gp286 cDNA (SEQ ID NO:1), gp286a cDNA
(SEQ ID NO:14) and genomic gp286 (SEQ ID NO:13) sequences

| Exon | gp286 cDNA | gp286a cDNA | genomic |
|---|---|---|---|
| exon 1 | 1-44 | 1-55 | 170-214 (gp286) |
| | | | 159-214 (gp286a) |
| exon 2 | 45-404 | 56-415 | 4209-4568 |
| exon 3 | 415-446 | 416-457 | 7738-7779 |
| exon 4 | 447-971 | — | 7874-8387 |
| exon 4a | — | 458-559 | 7874-7975 |
| exon 5 | — | 560-1065 | 9669-10174 |

Transcription Control Nucleic Acids

In another aspect, the present invention provides genome-derived isolated polynucleotides which include nucleic acid sequence elements that control transcription of the gp286 gene. These nucleic acids can be used, inter alia, to drive expression of heterologous coding regions in recombinant constructs, thus conferring upon such heterologous coding regions the expression pattern of the native gp286 gene. These nucleic acids can also be used, conversely, to target heterologous transcription control elements to the gp286 genomic locus, altering the expression pattern of the gp286 gene itself

In a first series of such embodiments, the invention provides an isolated polynucleotide comprising nucleotides 1-10500 of SEQ ID NO: 13; nucleotides 1-159 of SEQ ID NO: 13; nucleotides 1-170 of SEQ ID NO: 13; nucleotides 159-10500 of SEQ ID NO:13; nucleotides 170-10500 of SEQ ID NO: 13; nucleotides 4209-10500 of SEQ ID NO: 13; nucleotides 7738-10500 of SEQ ID NO: 13; nucleotides 7874-10500 of SEQ ID NO: 13 or nucleotides 9669-10500 of SEQ ID NO: 13; or the complements of such sequences, wherein the isolated nucleic acid is no more than about 100 kb in length, typically no more than about 75 kb in length, more typically no more than about 50 kb in length. Often, the isolated nucleic acids of these embodiments are no more than about 25 kb in length, often no more than about 15 kb in length, and frequently no more than about 10 kb in length. Even more preferably, the isolated polynucleotides are no more than 5000 base pairs, often no more than 1000 base pairs, 500 base pairs, 100 base pairs or 50 base pairs.

In some embodiments, the invention provides an isolated polynucleotide comprising at least 17, 18, 20, 24, or 25 nucleotides of nucleotides 1-10500 of SEQ ID NO:13; nucleotides 1-159 of SEQ ID NO:13; nucleotides 1-170 of SEQ ID NO:13; nucleotides 159-10500 of SEQ ID NO: 13; nucleotides 170-10500 of SEQ ID NO: 13; nucleotides 4209-10500 of SEQ ID NO: 13; nucleotides 7738-10500 of SEQ ID NO:13; nucleotides 7874-10500 of SEQ ID NO:13 or nucleotides 9669-10500 of SEQ ID NO: 13; or the complements of such sequences, wherein the isolated nucleic acid is no more than about 100 kb in length, typically no more than about 75 kb in length, more typically no more than about 50 kb in length. Often, the isolated nucleic acids of these embodiments are no more than about 25 kb in length, often no more than about 15 kb in length, and frequently no more than about 10 kb in length. Even more preferably, the isolated polynucleotides are no more than 5000 base pairs, often no more than 1000 base pairs, 500 base pairs, 100 base pairs or 50 base pairs.

Each of the isolated polynucleotides comprising nucleotides 1-10500 of SEQ ID NO:13; nucleotides 1-159 of SEQ ID NO: 13; nucleotides 1-170 of SEQ ID NO:13; nucleotides 159-10500 of SEQ ID NO:13; nucleotides 170-10500 of SEQ ID NO: 13; nucleotides 4209-10500 of SEQ ID NO: 13; nucleotides 7738-10500 of SEQ ID NO: 13; nucleotides 7874-10500 of SEQ ID NO: 13 or nucleotides 9669-10500 of SEQ ID NO: 13; comprises transcription control sequences that mediate developmental and tissue specific expression and regulation of the gp286 gene. Such transcription control sequences will be useful for conferring such developmental and tissue specific expression patterns on heterologous nucleic acid sequences operatively linked thereto.

Other Defining Features of gp286 Nucleic Acid Molecules

All the nucleic acid sequences specifically given herein are set forth as sequences of deoxyribonucleotides. It is intended, however, that the given sequences be interpreted as would be appropriate to the polynucleotide composition: for example, if the isolated nucleic acid is composed of RNA, the given sequence intends ribonucleotides, with uridine substituted for thymidine.

Polymorphisms such as single nucleotide polymorphisms (SNPs) occur frequently in eukaryotic genomes. More than 1.4 million SNPs have already identified in the human genome, International Human Genome Sequencing Consortium, Nature 409:860-921 (2001)—and the sequence determined from one individual of a species may differ from other allelic forms present within the population. Additionally, small deletions and insertions, rather than single nucleotide polymorphisms, are not uncommon in the general population, and often do not alter the function of the protein.

Accordingly, it is particularly emphasized that the present invention not only provides isolated polynucleotides identical in sequence to those described with particularity herein (e.g., SEQ ID Nos:1 and 14), but also to provide isolated polynucleotides that are allelic variants of those particularly described nucleic acid sequences. Further, the invention provides homologs (e.g., paralogs and orthologs) of gp286 that are at least about 65% identical in sequence to those described with particularity herein, typically at least about 70%, 75%, 80%, 85%, or 90% identical in sequence to those described with particularity herein, usefully at least about 91%, 92%, 93%, 94%, or 95% identical in sequence to those described with particularity herein, more usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention, as by random or directed mutagenesis.

For purposes herein, percent identity of two nucleic acid sequences is determined using the procedure of Tatiana et al, “Blast 2 sequences—a new tool for comparing protein and nucleotide sequences”, FEMS Microbiol Lett. 174:247-250 (1999), which procedure is effectuated by the computer program BLAST 2 SEQUENCES, available online at:

http://www.ncbi.nlm.nih.gov/Blast/b12seq/b12.html.

To assess percent identity of nucleic acid sequences, the BLASTN module of BLAST 2 SEQUENCES is used with default values of (i) reward for a match: 1; (i) penalty for a mismatch: −2; (iii) open gap 5 and extension gap 2 penalties; (iv) gap X_dropoff 50 expect 10 word size 11 filter, and both sequences are entered in their entireties.

The isolated polynucleotides of the present invention being useful for expression of GP286 proteins and protein fragments, the present invention thus provide isolated polynucleotides that encode GP286 proteins and portions thereof not only identical in sequence to those described with particularity herein, but degenerate variants thereof as well. As is well known, the genetic code is degenerate and codon choice for optimal expression varies from species to species. As is also well known, amino acid substitutions occur frequently among natural allelic variants, with conservative substitutions often occasioning only de minimis change in protein function.

Accordingly, the present invention provides polynucleotides not only identical in sequence to those described with particularity herein, but also those that encode GP286 and portions thereof, having conservative amino acid substitutions or moderately conservative amino acid substitutions.

Although there are a variety of metrics for calling conservative amino acid substitutions, based primarily on either observed changes among evolutionarily related proteins or on predicted chemical similarity, for purposes herein a conservative replacement is any change having a positive value in the PAM250 log-likelihood matrix reproduced herein below (see Gonnet et al., Science 256(5062):1443-5 (1992)):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 2 | −1 | 0 | 0 | 0 | 0 | 0 | 0 | −1 | −1 | −1 | 0 | −1 | −2 | 0 | 1 | 1 | −4 | −2 | 0 |
| R | −1 | 5 | 0 | 0 | −2 | 2 | 0 | −1 | 1 | −2 | −2 | 3 | −2 | −3 | −1 | 0 | 0 | −2 | −2 | −2 |
| N | 0 | 0 | 4 | 2 | −2 | 1 | 1 | 0 | 1 | −3 | −3 | 1 | −2 | −3 | −1 | 1 | 0 | −4 | −1 | −2 |
| D | 0 | 0 | 2 | 5 | −3 | 1 | 3 | 0 | 0 | −4 | −4 | 0 | −3 | −4 | −1 | 0 | 0 | −5 | −3 | −3 |
| C | 0 | −2 | −2 | −3 | 12 | −2 | −3 | −2 | −1 | −1 | −2 | −3 | −1 | −1 | −3 | 0 | 0 | −1 | 0 | 0 |
| Q | 0 | 2 | 1 | 1 | −2 | 3 | 2 | −1 | 1 | −2 | −2 | 2 | −1 | −3 | 0 | 0 | 0 | −3 | −2 | −2 |
| E | 0 | 0 | 1 | 3 | −3 | 2 | 4 | −1 | 0 | −3 | −3 | 1 | −2 | −4 | 0 | 0 | 0 | −4 | −3 | −2 |
| G | 0 | −1 | 0 | 0 | −2 | −1 | −1 | 7 | −1 | −4 | −4 | −1 | −4 | −5 | −2 | 0 | −1 | −4 | −4 | −3 |
| H | −1 | 1 | 1 | 0 | −1 | 1 | 0 | −1 | 6 | −2 | −2 | 1 | −1 | 0 | −1 | 0 | 0 | −1 | 2 | −2 |
| I | −1 | −2 | −3 | −4 | −1 | −2 | −3 | −4 | −2 | 4 | 3 | −2 | 2 | 1 | −3 | −2 | −1 | −2 | −1 | 3 |
| L | −1 | −2 | −3 | −4 | −2 | −2 | −3 | −4 | −2 | 3 | 4 | −2 | 3 | 2 | −2 | −2 | −1 | −1 | 0 | 2 |
| K | 0 | 3 | 1 | 0 | −3 | 2 | 1 | −1 | 1 | −2 | −2 | 3 | −1 | −3 | −1 | 0 | 0 | −4 | −2 | −2 |
| M | −1 | −2 | −2 | −3 | −1 | −1 | −2 | −4 | −1 | 2 | 3 | −1 | 4 | 2 | −2 | −1 | −1 | −1 | 0 | 2 |
| F | −2 | −3 | −3 | −4 | −1 | −3 | −4 | −5 | 0 | 1 | 2 | −3 | 2 | 7 | −4 | −3 | −2 | 4 | 5 | 0 |
| P | 0 | −1 | −1 | −1 | −3 | 0 | 0 | −2 | −1 | −3 | −2 | −1 | −2 | −4 | 8 | 0 | 0 | −5 | −3 | −2 |
| S | 1 | 0 | 1 | 0 | 0 | 0 | 0 | 0 | 0 | −2 | −2 | 0 | −1 | −3 | 0 | 2 | 2 | −3 | −2 | −1 |
| T | 1 | 0 | 0 | 0 | 0 | 0 | 0 | −1 | 0 | −1 | −1 | 0 | −1 | −2 | 0 | 2 | 2 | −4 | −2 | 0 |
| W | −4 | −2 | −4 | −5 | −1 | −3 | −4 | −4 | −1 | −2 | −1 | −4 | −1 | 4 | −5 | −3 | −4 | 14 | 4 | −3 |
| Y | −2 | −2 | −1 | −3 | 0 | −2 | −3 | −4 | 2 | −1 | 0 | −2 | 0 | 5 | −3 | −2 | −2 | 4 | 8 | −1 |
| V | 0 | −2 | −2 | −3 | 0 | −2 | −2 | −3 | −2 | 3 | 2 | −2 | 2 | 0 | −2 | −1 | 0 | −3 | −1 | 3 |

For purposes herein, a “moderately conservative” replacement is any change having a nonnegative value in the PAM250 log-likelihood matrix reproduced herein above.

To avoid severely reducing or eliminating biological activity, amino acid residues that are conserved among the GP286 proteins of various species or among the Ig family members are not altered (except by conservative substitution) during genetic engineering. For instance, the cysteine residues for maintaining an Ig domain of GP286 should be conserved.

Relatedness of polynucleotides can also be characterized using a functional test, the ability of the two polynucleotides to base-pair to one another at defined hybridization stringencies. The invention thus provides isolated polynucleotides not only identical in sequence to those described with particularity herein, but also to provide isolated polynucleotides (“cross-hybridizing nucleic acids”) that hybridize under high stringency conditions (as defined herein) to all or to a portion of various of the isolated gp286 polynucleotides of the present invention (“reference nucleic acids”).

Such cross-hybridizing nucleic acids are useful, inter alia, as probes for, and to drive expression of, proteins related to the proteins of the present invention such as alternative splice variants and homologs (e.g., orthologs and paralogs). Particularly useful orthologs are those from other primate species, such as chimpanzee, rhesus macaque monkey, baboon, orangutan, and gorilla; from rodents, such as rats, mice, guinea pigs; from lagomorphs, such as rabbits, and from domestic livestock, such as cow, pig, sheep, horse, goat.

The hybridizing portion of the reference nucleic acid is typically at least 15 nucleotides in length, and often at least 17, 20, 25, 30, 35, 40 or 50 nucleotides (nt) in length. Cross-hybridizing nucleic acids that hybridize to a larger portion of the reference nucleic acid—for example, to a portion of at least 50 nt, 100 nt, 150 nt, 200 nt, 250 nt, 300 nt, 350 nt, 400 nt, 450 nt, 500 nt or more, up to and including the entire length of the reference nucleic acid, are also useful.

The hybridizing portion of the cross-hybridizing nucleic acid is at least 75% identical in sequence to at least a portion of the reference nucleic acid. Typically, the hybridizing portion of the cross-hybridizing nucleic acid is at least 80%, often at least 85%, 86%, 87%, 88%, 89% or even at least 90% identical in sequence to at least a portion of the reference nucleic acid. Often, the hybridizing portion of the cross-hybridizing nucleic acid will be at least 91%, 92%, 93%, 94%, 95%, 96%, 97%, 98%, or 99% identical in sequence to at least a portion of the reference nucleic acid sequence. At times, the hybridizing portion of the cross-hybridizing nucleic acid will be at least 99.5% identical in sequence to at least a portion of the reference nucleic acid.

The invention also provides fragments of various of the isolated polynucleotides or nucleic acids of the present invention. By “fragments” of a reference nucleic acid is here intended isolated polynucleotides or nucleic acids, however obtained, that have a nucleotide sequence identical to a portion of the reference nucleic acid sequence, which portion is at least 17 nucleotides and less than the entirety of the reference nucleic acid.

In theory, an oligonucleotide of 17 nucleotides is of sufficient length as to occur at random less frequently than once in the three gigabases of the human genome, and thus to provide a nucleic acid probe that can uniquely identify the reference sequence in a nucleic acid mixture of mammalian genomic complexity. Further specificity can be obtained by probing nucleic acid samples of subgenomic complexity, and/or by using plural fragments as short as 17 nucleotides in length collectively to prime amplification of nucleic acids, as, e.g., by polymerase chain reaction (PCR).

The nucleic acid probes of the invention can be used to detect RNA transcripts or genomic sequences encoding homologs or identical proteins. The probe may comprise a label group attached thereto, e.g., a radioisotope, a fluorescent compound, an enzyme, or an enzyme co-factor. Such probes can be used as a part of diagnostic kit for identifying cells or tissues (i) that mis-express a GP286 protein (e.g., aberrant splicing, abnormal mRNA levels), or (ii) that harbor a mutation in the gp286 gene, such as a deletion, an insertion, or a point mutation. Such diagnostic kits preferably include labeled reagents and instructional inserts for their use.

The isolated polynucleotides of the invention can also be used as primers in PCR, primer extension and the like. To be useful as primers, the polynucleotides can be, e.g., at least 6 nucleotides (e.g., at least 7, 8, 9, or 10) in length. The primers can hybridize to an exonic sequence of a gp286 gene, for, e.g., amplification of a gp286 mRNA or cDNA Alternatively, the primers can hybridize to an intronic sequence or an upstream or downstream regulatory sequence of a gp286 gene, to utilize non-transcribed, e.g., regulatory portions of the genomic structure of a gp286 gene.

The nucleic acid primers of the present invention can also be used, for example, to prime single base extension (SBE) for SNP detection (see, e.g., U.S. Pat. No. 6,004,744, the disclosure of which is incorporated herein by reference in its entirety). Isothermal amplification approaches, such as rolling circle amplification, are also now well-described. See, e.g., Schweitzer et al., Curr. Opin. Biotechnol. 12(1):21-7 (2001); U.S. Pat. Nos. 5,854,033 and 5,714,320 and international patent publications WO 97/19193 and WO 00/15779, the disclosures of which are incorporated herein by reference in their entireties. Rolling circle amplification can be combined with other techniques to facilitate SNP detection. See, e.g., Lizardi et al., Nature Genet. 19(3):225-32 (1998).

As described below, nucleic acid fragments that encode at least 6 contiguous amino acids (i.e., fragments of 18 nucleotides or more) are useful in directing the expression or the synthesis of peptides that have utility in mapping the epitopes of the protein encoded by the reference nucleic acid. See, e.g., Geysen et al., Proc. Natl. Acad. Sci. USA 81:3998-4002 (1984); and U.S. Pat. Nos. 4,708,871 and 5,595,915.

And, as described below, nucleic acid fragments that encode at least 8 contiguous amino acids (i.e., fragments of 24 nucleotides or more) are useful in directing the expression or the synthesis of peptides that have utility as immunogens. See, e.g., Lerner, “Tapping the immunological repertoire to produce antibodies of predetermined specificity,” Nature 299:592-596 (1982); Shinnick et al., Annu. Rev. Microbiol. 37:425-46 (1983); Sutcliffe et al., Science 219:660-6 (1983).

The nucleic acid fragment of the present invention is thus at least 17 nucleotides in length, typically at least 18 nucleotides in length, and often at least 24, 25, 30, 35, 40, or 45 nucleotides (nt) in length. Of course, larger fragments having at least 50 nt, 100 nt, 150 nt, 200 nt, 250 nt, 300 nt, 350 nt, 400 nt, 450 nt, 500 nt or more are also useful, and at times preferred, as will be appreciated by the skilled worker.

Having been based upon the mining of genomic sequence, rather than upon surveillance of expressed message, the present invention further provides isolated genome-derived polynucleotides or nucleic acids that include portions of the gp286 gene. The invention particularly provides genome-derived single exon probes, which comprise at least part of an exon (“reference exon”) and can hybridize detectably under high stringency conditions to transcript-derived nucleic acids that include the reference exon. The single exon probe will not, however, hybridize detectably under high stringency conditions to nucleic acids that lack the reference exon but include one or more exons that are found adjacent to the reference exon in the genome.

The present invention also provides isolated genome-derived polynucleotides or nucleic acids which include nucleic acid sequence elements that control transcription of the gp286 gene. Transcription control sequences include, e.g., promoters, enhancers, operators, terminators, silencers, and the like.

When desired for use in antisense inhibition of transcription or translation, or for antisense-mediated targeting of enzymatic nucleic acid molecules such as ribozymes, the isolated polynucleotides and nucleic acids of the present invention can usefully include one or more modified bases (see below) and/or one or more modified or altered internucleoside bonds, which often provide nuclease-resistance. See Hartmann et al. (eds.), Manual of Antisense Methodology (Perspectives in Antisense Science), Kluwer Law International (1999) (ISBN:079238539X); Stein et al. (eds.), Applied Antisense Oligonucleotide Technology, Wiley-Liss (cover (1998) (ISBN: 0471172790); Chadwick et al. (eds.), Oligonucleotides as Therapeutic Agents—Symposium No. 209, John Wiley & Son Ltd (1997) (ISBN: 0471972797). Such altered bases and internucleoside bonds are often desired also when the isolated nucleic acid of the present invention is to be used for targeted gene correction, as described in Gamper et al., Nucl. Acids Res. 28(21):4332-9 (2000), the disclosure of which is incorporated herein by reference in its entirety.

The antisense nucleic acid molecules (and enzymatic nucleic acids targeted by antisense) of the invention can be used in a therapeutic setting. These molecules can be expressed from an expression vector that contains an operably linked transcription regulatory sequence, the activity of which can be determined by the cell type into which the vector is introduced. For a discussion of the regulation of gene expression using antisense genes, see Weintraub et al., Antisense RNA as a molecular tool for genetic analysis, REVIEWS—TRENDS IN GENETICS, Vol. 1(1) (1986).

An antisense nucleic acid of the invention may be a ribozyme. Ribozymes are catalytic RNA molecules with ribonuclease activity that are capable of cleaving a single-stranded nucleic acid, such as an mRNA, to which they have a complementary region. Thus, ribozymes can be used to catalytically cleave gp286 mRNA transcripts to thereby inhibit translation of gp286 mRNA. A ribozyme having specificity for a gp286-encoding nucleic acid can be designed based upon the nucleotide sequence of a gp286 polynucleotide disclosed herein (i.e., SEQ ID NOs:1, 13 or 14).

Oligonucleotide mimetics of gp286, such as peptide nucleic acids (PNA), can be used in therapeutic and diagnostic applications. See, e.g., Hyrup et al. (1996) Bioorg. Med. Chem. Lett. 4:5-23. In PNA compounds, the phosphodiester backbone of the nucleic acid is replaced with an amide-containing backbone, in particular by repeating N-(2-aminoethyl) glycine units linked by amide bonds. PNAs For example, PNAs can be used as antisense or antigene agents for sequence-specific modulation of gene expression by, e.g., inducing transcription or translation arrest or inhibiting replication. PNAs of gp286 can also be used, e.g., in the analysis of single base pair mutations in a gene by, e.g., PNA directed PCR clamping; as artificial restriction enzymes when used in combination with other enzymes, e.g., S1 nucleases; or as probes or primers for DNA sequence and hybridization (Hyrup et al., supra; and Perry-O'Keefe, supra). PNAs of gp286 can be modified, e.g., to enhance their stability or cellular uptake, by attaching lipophilic or other helper groups to PNA, by the formation of PNA-DNA chimeras, or by the use of liposomes or other techniques of drug delivery known in the art (see infra).

Oligonucleotide of the invention may include other appended groups such as peptides (e.g., for targeting host cell receptors in vivo), or agents facilitating transport across the cell membrane or the blood-brain barrier. In addition, oligonucleotides can be modified with hybridization triggered cleavage agents or intercalating agents. To this end, the oligonucleotide may be conjugated to another molecule, e.g., a peptide, a hybridization triggered cross-linking agent, a transport agent, a hybridization-triggered cleavage agent, etc. (see infra).

Differences from nucleic acid compositions found in nature—e.g. non-native bases, altered internucleoside linkages, post-synthesis modification—can be present throughout the length of the gp286 polynucleotide or can usefully be localized to discrete portions thereof As an example of the latter, chimeric nucleic acids can be synthesized that have discrete DNA and RNA domains and demonstrated utility for targeted gene repair, as further described in U.S. Pat. Nos. 5,760,012 and 5,731,181, the disclosures of which are incorporated herein by reference in their entireties. Chimeric nucleic acids comprising both DNA and PNA have been demonstrated to have utility in modified PCR reactions. See Misra et al., Biochem. 37: 1917-1925 (1998); see also Finn et al., Nucl Acids Res. 24: 3357-3363 (1996), incorporated herein by reference.

Polynucleotides and nucleic acids of the present invention can also usefully be bound to a substrate. The substrate can porous or solid, planar or non-planar, unitary or distributed; the bond can be covalent or noncovalent. Bound to a substrate, nucleic acids of the present invention can be used as probes in their unlabeled state. For example, the nucleic acids of the present invention can usefully be bound to a porous substrate, commonly a membrane, typically comprising nitrocellulose, nylon, or positively-charged derivatized nylon; so attached, the nucleic acids of the present invention can be used to detect gp286 nucleic acids present within a labeled nucleic acid sample, either a sample of genomic nucleic acids or a sample of transcript-derived nucleic acids, e.g. by reverse dot blot.

The nucleic acids of the present invention can also usefully be bound to a solid substrate, such as glass, although other solid materials, such as amorphous silicon, crystalline silicon, or plastics, can also be used. The nucleic acids of the present invention can be attached covalently to a surface of the support substrate or applied to a derivatized surface in a chaotropic agent that facilitates denaturation and adherence by presumed noncovalent interactions, or some combination thereof.

The nucleic acids of the present invention can be bound to a substrate to which a plurality of other nucleic acids are concurrently bound, hybridization to each of the plurality of bound nucleic acids being separately detectable. At low density, e.g. on a porous membrane, these substrate-bound collections are typically denominated macroarrays; at higher density, typically on a solid support, such as glass, these substrate bound collections of plural nucleic acids are colloquially termed microarrays. As used herein, the term microarray includes arrays of all densities. The invention thus provides microarrays that include the nucleic acids of the present invention.

The isolated nucleic acids of the present invention can be used as hybridization probes to detect, characterize, and quantify gp286 nucleic acids in, and isolate gp286 nucleic acids from, both genomic and transcript-derived nucleic acid samples. When free in solution, such probes are typically, but not invariably, detectably labeled; bound to a substrate, as in a microarray, such probes are typically, but not invariably unlabeled.

For example, the isolated nucleic acids of the present invention can be used as probes to detect and characterize gross alterations in the gp286 genomic locus, such as deletions, insertions, translocations, and duplications of the gp286 genomic locus through fluorescence in situ hybridization (FISH) to chromosome spreads. See, e.g., Andreeff et al. (eds.), Introduction to Fluorescence In Situ Hybridization: Principles and Clinical Applications, John Wiley & Sons (1999) (ISBN: 0471013455), the disclosure of which is incorporated herein by reference in its entirety. The isolated nucleic acids of the present invention can be used as probes to assess smaller genomic alterations using, e.g., Southern blot detection of restriction fragment length polymorphisms. The isolated nucleic acids of the present invention can be used as probes to isolate genomic clones that include the nucleic acids of the present invention, which thereafter can be restriction mapped and sequenced to identify deletions, insertions, translocations, and substitutions (single nucleotide polymorphisms, SNPs) at the sequence level.

The isolated nucleic acids of the present invention can be also be used as probes to detect, characterize, and quantify gp286 nucleic acids in, and isolate gp286 nucleic acids from, transcript-derived nucleic acid samples. For example, the isolated nucleic acids of the present invention can be used as hybridization probes to detect, characterize by length, and quantify gp286 mRNA by northern blot of total or poly-A⁺-selected RNA samples. The isolated nucleic acids of the present invention can also be used as hybridization probes to detect, characterize by location, and quantify gp286 message by in situ hybridization to tissue sections (see, e.g., Schwarchzacher et al., In Situ Hybridization, Springer-Verlag New York (2000) (ISBN: 0387915966), the disclosure of which is incorporated herein by reference in its entirety).

Further, the isolated nucleic acids of the present invention can be used as hybridization probes to measure the representation of gp286 clones in a cDNA library. For example, the isolated nucleic acids of the present invention can be used as hybridization probes to isolate gp286 nucleic acids from cDNA libraries, permitting sequence level characterization of gp286 RNA messages, including identification of deletions, insertions, truncations—including deletions, insertions, and truncations of exons in alternatively spliced forms—and single nucleotide polymorphisms.

As described in the Examples herein below, the nucleic acids of the present invention can also be used to detect and quantify gp286 nucleic acids in transcript-derived samples to measure expression of the gp286 gene. Measurement of gp286 expression has particular utility in diagnostic assays for conditions, disorders and diseases associated with abnormal gp286 expression, particularly in T cells, where gp286 is normally expressed, as well as in tissues where it may be mis-expressed, as further described in the Examples herein below.

As would be readily apparent to one of skill in the art, each gp286 nucleic acid probe—whether labeled, substrate-bound, or both—is thus currently available for use as a tool for measuring the level of gp286 expression in immune and other tissues, in which expression has already been confirmed.

The gp286 nucleic acid probes of the present invention are also useful for determining levels of T cell infiltration into tissues. This may be particularly useful in determining whether a tissue is inflamed, in which case T cells will infiltrate the tissue, or in other disease states in which T cell infiltration occurs. One having ordinary skill in the art may compare the level of gp286 mRNA in a normal tissue to that of a sample tissue suspected of being inflamed or diseased, wherein a determination that the gp286 mRNA level is higher in the sample tissue would be indicative of T cell infiltration and therefore of inflammation or other disease state.

As noted above, the nucleic acid probes of the present invention are useful in constructing microarrays; the microarrays, in turn, are products of manufacture that are useful for measuring and for surveying gene expression in, for example, drug discovery and target validation programs. When included on a microarray, each gp286 nucleic acid probe makes the microarray specifically useful for detecting that portion of the gp286 gene included within the probe, thus imparting upon the microarray device the ability to detect a signal where, absent such probe, it would have reported no signal.

Changes in the level of gp286 expression need not be observed for the measurement of expression to have utility. Where gene expression analysis is used to assess toxicity of chemical agents on cells, for example, the failure of the agent to change a gene's expression level is evidence that the drug likely does not affect the pathway of which the gene's expressed protein is a part. Analogously, where gene expression analysis is used to assess side effects of pharmacologic agents—whether in lead compound discovery or in subsequent screening of lead compound derivatives—the inability of the agent to alter a gene's expression level is evidence that the drug does not affect the pathway of which the gene's expressed protein is a part. WO 99/58720, incorporated herein by reference in its entirety, provides methods for quantifying the relatedness of a first and second gene expression profile and for ordering the relatedness of a plurality of gene expression profiles, without regard to the identity or function of the genes whose expression is used in the calculation.

The genome-derived single exon probes and genome-derived single exon probe microarrays of the invention have the additional utility of permitting high-throughput detection of splice variants of the nucleic acids of the present invention.

Polynucleotides of the present invention, inserted into nucleic acid constructs such as vectors which flank the polynucleotide insert with a promoter can be used to drive in vitro expression of RNA complementary to either strand of the nucleic acid of the present invention. The RNA can be used as a single-stranded probe, in cDNA-mRNA subtraction, or for in vitro translation. Those polynucleotides which encode GP286 protein or portions thereof can further be used to express the GP286 proteins or protein fragments, either alone, or as part of fusion proteins. Expression can be from genomic or transcript-derived polynucleotides of the present invention.

Where protein expression is effected from genomic DNA, expression will typically be effected in eukaryotic, typically mammalian, cells capable of splicing introns from the initial RNA transcript. Expression can be driven from episomal vectors or from genomic DNA integrated into a host cell chromosome. As described below, where expression is from transcript-derived (or otherwise intron-less) polynucleotides of the invention, expression can be effected in a wide variety of prokaryotic or eukaryotic cells.

Expressed in vitro, the protein, protein fragment, or protein fusion can thereafter be isolated, to be used as a standard in immunoassays specific for the proteins, or protein isoforms, of the present invention; to be used as a therapeutic agent, e.g., to be administered as passive replacement therapy in individuals deficient in the proteins of the present invention; to be administered as a vaccine; to be used for in vitro production of specific antibody, the antibody thereafter to be used, e.g., as an analytical reagent for detection and quantitation of the proteins of the present invention or to be used as an immunotherapeutic agent.

The isolated polynucleotides and nucleic acids of the present invention can also be used to drive in vivo expression of the proteins of the present invention. In vivo expression can be driven from a vector—typically a viral vector, often a vector based upon a replication incompetent lentivirus, retrovirus, adenovirus, or adeno-associated virus (AAV)—for purpose of gene therapy. In vivo expression can be driven from expression control signals endogenous or exogenous (e.g., from a vector) to the nucleic acid.

Various forms of the isolated gp286 polynucleotides of the invention (e.g., genomic or cDNA) can be microinjected into male or female pronuclei, or can be integrated into embryonic stem (ES) cells to create transgenic non-human animals capable of producing the proteins of the present invention.

Genomic nucleic acids of the present invention can also be used to target homologous recombination to a gp286 locus in a subject. See, e.g., U.S. Pat. Nos. 6,187,305; 6,204,061; 5,631,153; 5,627,059; 5,487,992; 5,464,764; 5,614,396; 5,527,695 and 6,063,630; and Kmiec et al. (eds.), Gene Targeting Protocols, Vol. 133, Humana Press (2000) (ISBN: 0896033600); Joyner (ed.), Gene Targeting: A Practical Approach, Oxford University Press, Inc. (2000) (ISBN: 0199637938); Sedivy et al., Gene Targeting, Oxford University Press (1998) (ISBN: 071677013X); Tymms et al. (eds.), Gene Knockout Protocols, Humana Press (2000) (ISBN: 0896035727); Mak et al. (eds.), The Gene Knockout FactsBook, Vol. 2, Academic Press, Inc. (1998) (ISBN: 0124660444); Torres et al., Laboratory Protocols for Conditional Gene Targeting, Oxford University Press (1997) (ISBN: 019963677X); Vega (ed.), Gene Targeting, CRC Press, LLC (1994) (ISBN: 084938950X), the disclosures of which are incorporated herein by reference in their entireties.

Where the genomic region includes transcription regulatory elements, homologous recombination can be used to alter the expression of GP286, both for purpose of in vitro production of GP286 protein from human cells, and for purposes of gene therapy. See, e.g., U.S. Pat. Nos. 5,981,214, 6,048,524; 5,272,071; the disclosures of which are incorporated herein by reference in their entireties. Fragments of the polynucleotides of the present invention smaller than those typically used for homologous recombination can also be used for targeted gene correction or alteration, possibly by cellular mechanisms different from those engaged during homologous recombination. See, e.g., U.S. Pat. Nos. 5,945,339, 5,888,983, 5,871,984, 5,795,972, 5,780,296, 5,760,012, 5,756,325, 5,731,181; and Culver et al., “Correction of chromosomal point mutations in human cells with bifunctional oligonucleotides,” Nature Biotechnol. 17(10):989-93 (1999); Gamper et al., Nucl. Acids Res. 28(21):4332-9 (2000), the disclosures of which are incorporated herein by reference.

Polynucleotides of the present invention can be obtained by using the labeled probes of the present invention to probe nucleic acid samples, such as genomic libraries, cDNA libraries, and mRNA samples, by standard techniques. Polynucleotides of the present invention can also be obtained by amplification, using the nucleic acid primers of the present invention, as further demonstrated in Example 1, herein below. Polynucleotides of the present invention, especially if fewer than about 100 nucleotide, can also be synthesized chemically, typically by solid phase synthesis using commercially available automated synthesizers.

Vectors and Host Cells

A. Nucleic Acid Constructs

The present invention provides nucleic acid constructs, such as vectors, that comprise one or more of the isolated polynucleotides of the invention, and host cells into which such vectors have been introduced.

The vectors can be used for propagating the polynucleotides of the present invention in host cells (cloning vectors), for shuttling the polynucleotides of the present invention between host cells derived from disparate organisms (shuttle vectors), for inserting the polynucleotides of the present invention into host cell chromosomes (insertion vectors), for expressing sense or antisense RNA transcripts of the polynucleotides of the present invention in vitro or within a host cell, and for expressing polypeptides encoded by the polynucleotides of the present invention, alone or as fusions to heterologous polypeptides (expression vectors). Vectors of the present invention will often be suitable for several such uses.

Vectors are by now well-known in the art, and are described, inter alia, in Jones et al. (eds.), Vectors: Cloning Applications: Essential Techniques (Essential Techniques Series), John Wiley & Son Ltd 1998 (ISBN: 047196266X); Jones et al. (eds.), Vectors: Expression Systems: Essential Techniques (Essential Techniques Series), John Wiley & Son Ltd, 1998 (ISBN:0471962678); Gacesa et al., Vectors: Essential Data, John Wiley & Sons, 1995 (ISBN: 0471948411); Cid-Arregui (eds.), Viral Vectors: Basic Science and Gene Therapy, Eaton Publishing Co., 2000 (ISBN: 188129935X); Sambrook et al., Molecular Cloning: A Laboratory Manual (3^{rd} ed.), Cold Spring Harbor Laboratory Press, 2001 (ISBN: 0879695773); Ausubel et al (eds.), Short Protocols in Molecular Biology: A Compendium of Methods from Current Protocols in Molecular Biology (4^{th} ed.), John Wiley & Sons, 1999 (ISBN: 047132938X), the disclosures of which are incorporated herein by reference in their entireties. An enormous variety of vectors are available commercially. Use of existing vectors and modifications are well within the skill in the art.

Typically, vectors are derived from virus, plasmid, prokaryotic or eukaryotic chromosomal elements, or some combination thereof, and include at least one origin of replication, at least one site for insertion of heterologous nucleic acid, typically in the form of a polylinker with multiple, tightly clustered, single cutting restriction sites, and at least one selectable marker, although some integrative vectors will lack an origin that is functional in the host to be chromosomally modified, and some vectors will lack selectable markers. Vectors of the invention will further include at least one isolated polynucleotide nucleic acid of the invention inserted into the vector in at least one location. Where present, the origin of replication and selectable markers are chosen based upon the desired host cell or host cells; the host cells, in turn, are selected based upon the desired application.

For example, prokaryotic cells, typically E. coli, are typically chosen for cloning, i.e., for amplification of polynucleotide sequences in a host cell. In such case, vector replication is predicated on the replication strategies of coliform-infecting phage—such as phage lambda, M13, T7, T3 and P1—or on the replication origin of autonomously replicating episomes, notably the ColE1 plasmid and later derivatives, including pBR322 and the pUC series plasmids. Where E. coli is used as host, selectable markers are, analogously, chosen for selectivity in gram negative bacteria: e.g., typical markers confer resistance to antibiotics, such as ampicillin, tetracycline, chloramphenicol, kanamycin, streptomycin, zeocin; auxotrophic markers can also be used.

As another example, yeast cells, typically S. cerevisiae, are chosen, inter alia, for eukaryotic genetic studies, for identification of interacting protein components, e.g. through use of a two-hybrid system, and for protein expression. Vectors of the present invention for use in yeast will typically, but not invariably, contain an origin of replication suitable for use in yeast and a selectable marker that is functional in yeast. Examples of suitable yeast vectors include integrative YIp vectors, replicating episomal YEp vectors containing centromere sequences, CEN, and autonomously replicating sequences, ARS. YACs are based on yeast linear plasmids, denoted YLp, containing homologous or heterologous DNA sequences that function as telomeres (TEL) in vivo, as well as containing yeast ARS (origins of replication) and CEN (centromeres) segments.

Selectable markers in yeast vectors include a variety of auxotrophic markers, the most common of which are (in Saccharomyces cerevisiae) URA3, HIS3, LEU2, TRP1 and LYS2, which complement specific auxotrophic mutations, such as ura3-52, his3-D1, leu2-D1, trpl-D1 and lys2-201. The URA3 and LYS2 yeast genes further permit negative selection based on specific inhibitors, 5-fluoro-orotic acid (BOA) and α-aminoadipic acid (αAA), respectively, that prevent growth of the prototrophic strains but allows growth of the ura3 and lys2 mutants, respectively. Other selectable markers confer resistance to, e.g., zeocin.

Insect cells are often chosen for high efficiency protein expression. Where the host cells are from Spodoptera frugiperda—e.g., Sf9 and Sf21 cell lines, and expresSF™ cells (Protein Sciences Corp., Meriden, Conn., USA)—the vector replicative strategy is typically based upon the baculovirus life cycle. Typically, baculovirus transfer vectors are used to replace the wild-type AcMNPV polyhedrin gene with a heterologous gene of interest. Sequences that flank the polyhedrin gene in the wild-type genome are positioned 5′ and 3′ of the expression cassette on the transfer vectors. Following cotransfection with AcMNPV DNA, a homologous recombination event occurs between these sequences resulting in a recombinant virus carrying the gene of interest and the polyhedrin or p10 promoter. Selection can be based upon visual screening for lacZ fusion activity.

Mammalian cells are often chosen for expression of proteins intended as pharmaceutical agents, and are also chosen as host cells for screening of potential agonist and antagonists of a protein or a physiological pathway. Vectors intended for autonomous extrachromosomal replication in mammalian cells will typically include a viral origin, such as the SV40 origin (for replication in cell lines expressing the large T-antigen, such as COS1 and COS7 cells), the papillomavirus origin, or the EBV origin for long term episomal replication (for use, e.g., in 293-EBNA cells, which constitutively express the EBV EBNA-1 gene product and adenovirus E1A). Vectors intended for integration, and thus replication as part of the mammalian chromosome, can, but need not, include an origin of replication functional in mammalian cells, such as the SV40 origin. Vectors based upon viruses, such as lentiviruses, adenovirus, adeno-associated virus, vaccinia virus, and various mammalian retroviruses, will typically replicate according to the viral replicative strategy.

Selectable markers for use in mammalian cells include resistance to neomycin (G418), blasticidin, hygromycin and to zeocin, and selection based upon the purine salvage pathway using HAT medium.

Plant cells can also be used for expression, with the vector replicon typically derived from a plant virus (e.g., cauliflower mosaic virus, CaMV; tobacco mosaic virus, TMV) and selectable markers chosen for suitability in plants.

For propagation of polynucleotides of the present invention that are larger than can readily be accomodated in vectors derived from plasmids or virus, the invention further provides artificial chromosomes—BACs, YACs, and HACs—that comprise gp286 nucleic acids, often genomic nucleic acids. See, e.g., Shizuya et al., Keio J. Med 50(1):26-30 (2001); Shizuya et al., Proc. Natl. Acad. Sci. USA 89(18):8794-7 (1992); Kuroiwa et al, Nature Biotechnol. 18(10):1086-90 (2000); Henning et al., Proc. Natl. Acad. Sci. USA 96(2):592-7 (1999); Harrington et al., Nature Genet. 15(4):345-55 (1997), the disclosures of which are incorporated herein by reference.

Expression vectors of the invention which will drive expression of polypeptides from the inserted heterologous nucleic acid will often include a variety of other genetic elements operatively linked to the protein-encoding heterologous nucleic acid insert, typically genetic elements that drive and regulate transcription, such as promoters and enhancer elements, those that facilitate RNA processing, such as transcription termination, splicing signals and/or polyadenylation signals, and those that facilitate translation, such as ribosomal consensus sequences. Other transcription control sequences include, e.g., operators, silencers, and the like. Use of such expression control elements, including those that confer inducible expression, and developmental or tissue-regulated expression are well-known in the art.

Tissue-specific regulatory elements capable of expressing GP286 in the immune system may be particularly useful and are known in the art, e.g., native immunoglobulin promoters and native T cell receptor promoters. Developmentally-regulated promoters may also be selected, including but not limited to the murine hox promoters (Kessel and Gruss (1990) Science 249:374-379) and the α-fetoprotein promoter (Campes and Tilghman (1989) Genes Dev. 3:537-546). A huge variety of inducible promoters are known and may be selected based on the particular application.

Expression vectors can be designed to fuse the expressed polypeptide to small protein tags that facilitate purification and/or visualization. Many such tags are known and available. Expression vectors can also be designed to fuse proteins encoded by the heterologous nucleic acid insert to polypeptides larger than purification and/or identification tags. Useful protein fusions include those that permit display of the encoded protein on the surface of a phage or cell, fusions to intrinsically fluorescent proteins, such as luciferase or those that have a green fluorescent protein (GFP)-like chromophore, fusions to the IgG Fc region or other immunoglobulin type constant domains, and fusions for use in two hybrid selection systems.

For secretion of expressed proteins, a wide variety of vectors are available which include appropriate sequences that encode secretion signals, such as leader peptides. Vectors designed for phage display, yeast display, and mammalian display, for example, target recombinant proteins using an N-terminal cell surface targeting signal and a C-terminal transmembrane anchoring domain.

A wide variety of vectors now exist that fuse proteins encoded by heterologous nucleic acids to the chromophore of the substrate-independent, intrinsically fluorescent green fluorescent protein from Aequorea victoria (“GFP”) and its many color-shifted and/or stabilized variants.

Vectors which allow fusions of heterologous sequences to the IgG Fc region to increase serum half-life of protein pharmaceutical products through interaction with the FcRn receptor (also denominated the FcRp receptor and the Brambell receptor, FcRb), are also widely available.

For long-term, high-yield recombinant production of the proteins, protein fusions, and protein fragments of the present invention, stable expression is preferred. Stable expression is readily achieved by integration into the host cell genome of vectors (preferably having selectable markers), followed by selection for integrants.

Vectors of the invention will also often include elements that permit in vitro transcription of RNA from the inserted heterologous nucleic acid. Such vectors typically include a phage promoter, such as that from T7, T3, or SP6, flanking the nucleic acid insert. Often two different such promoters flank the inserted nucleic acid, permitting separate in vitro production of both sense and antisense strands.

B. Host Cells

The present invention further includes host cells—either prokaryotic (bacteria) or eukaryotic (e.g., yeast, insect, plant and animal cells)—comprising the nucleic acid constructs such as vectors of the present invention, either present episomally within the cell or integrated, in whole or in part, into the host cell chromosome.

Among other considerations, some of which are described above, a host cell strain may be chosen for its ability to process the expressed protein in the desired fashion. Such post-translational modifications of the polypeptide include, but are not limited to, acetylation, carboxylation, glycosylation, phosphorylation, lipidation, and acylation, and it is an aspect of the present invention to provide GP286 proteins with such post-translational modifications.

Representative, non-limiting examples of appropriate host cells include bacterial cells, such as E. coli, Caulobacter crescentus, Streptomyces species, and Salmonella typhimurium; yeast cells, such as Saccharomyces cerevisiae, Schizosaccharomyces pombe, Pichia pastoris, Pichia methanolica; insect cell lines, such as those from Spodoptera frugiperda—e.g., Sf9 and Sf21 cell lines, and expresSF cells (Protein Sciences Corp., Meriden, Conn., USA)—Drosophila S2 cells, and Trichoplusia ni High Five® Cells (Invitrogen, Carlsbad, Calif., USA); and mammalian cells. Typical mammalian cells include COS1 and COS7 cells, chinese hamster ovary (CHO) cells, NIH 3T3 cells, 293 cells, HEPG2 cells, HeLa cells, L cells, HeLa, MDCK, HEK293, WI38, murine ES cell lines (e.g., from strains 129/SV, C57/BL6, DBA-1, 129/SVJ), K562, Jurkat cells, and BW5147. Particularly preferred are T cell lines, preferably human T cell lines, e.g., MOLT-4. Other useful mammalian cell lines are well known and readily available from the American Type Culture Collection (ATCC) (Manassas, Va., USA) and the National Institute of General medical Sciences (NIGMS) Human Genetic Cell Repository at the Coriell Cell Repositories (Camden, N.J., USA).

Methods for introducing the vectors and nucleic acids of the present invention into the host cells are well known in the art; the choice of technique will depend primarily upon the specific vector to be introduced and the host cell chosen.

GP286 Proteins, Polypeptides and Fragments

The present invention provides GP286 proteins and various fragments thereof suitable for use as antigens (e.g., for epitope mapping), for use as immunogens (e.g., for raising antibodies or as vaccines), and for use in therapeutic compositions. Also provided are fusions of GP286 polypeptides and fragments to heterologous polypeptides, and conjugates of the proteins, fragments, and fusions of the present invention to other moieties (e.g., to carrier proteins, to fluorophores).

In some embodiments, the invention provides an isolated GP286 polypeptide comprising the amino acid sequence encoded by a full-length gp286 cDNA (SEQ ID NOs:1 and 14), or a degenerate variant. The invention also provides an isolated GP286 polypeptide having the amino acid sequence encoded by a full-length gp286 cDNA (SEQ ID NOs:1 and 14), optionally having one or more conservative amino acid substitutions.

The invention also provides an isolated GP286 polypeptide comprising the amino acid sequence encoded by a polynucleotide sequence that hybridizes under high stringency conditions to a probe having part or all of the nucleotide sequence of a gp286 cDNA (SEQ ID NOs:1 and 14). Preferably, an isolated GP286 polypeptide encoded by a stringently cross-hybridizing polynucleotide of the invention will have at least one biological activity of GP286.

In another series of embodiments, the invention provides an isolated GP286 polypeptide comprising the GP286 amino acid sequence of either SEQ ID NOs:2 or 15, optionally having one or more conservative amino acid substitutions. Also provided is an isolated GP286 polypeptide having the amino acid sequence encoded by the full-length GP286 polypeptide sequence of either SEQ ID NOs:2 or 15, optionally having one or more conservative amino acid substitutions.

The invention further provides fragments of each of the above-described isolated polypeptides, particularly fragments having at least 6 amino acids, 8 amino acids, 15 amino acids up to the entirety of the sequence given in either SEQ ID NOs:2 or 15.

The invention also provides an isolated GP286 polypeptide comprising all or a portion of the N-terminal extracellular domain of GP286. (See FIGS. 1 and 2 and SEQ ID NOs:2 and 15 for GP286 domains and sequences). Preferred polypeptides are those having the amino acid sequences of SEQ ID NO: 21, which is the Ig-like domain of GP286, and SEQ ID NO: 27, which is the extracellular domain of GP286 without the amino acids N-terminal of the Ig-like domain.

In preferred embodiments, the isolated GP286 polypeptide comprises the entire extracellular domain of GP286 and lacks a functional GP286 transmembrane domain. Preferred polypeptides are those having the amino acid sequences of SEQ ID NOs: 17 and 19, which are the extracellular portions of GP286 and GP286a, respectively. In other preferred embodiments, the isolated GP286 polypeptide consists of part or all of the GP286 extracellular domain fused to a heterologous protein domain.

Also preferred is an isolated GP286 polypeptide comprising a GP286 fragment selected from the group consisting of the transmembrane domain of GP286 and the C-terminal cytoplasmic region of GP286. Preferred polypeptides are those having the amino acid sequences of SEQ ID NO: 33, which is the transmembrane domain of GP286; SEQ ID NO: 23, which is the intracellular domain of GP286; and SEQ ID NO: 25; which is the intracellular domain of GP286a. In other preferred embodiments, the isolated GP286 polypeptide consists of part or all of the GP286 cytoplasmic or transmembrane domains fused to a heterologous protein domain.

The GP286 fragments of the invention may be continuous portions of the native GP286 protein. However, it will be appreciated that knowledge of the GP286 gene and protein sequences as provided herein permits recombining of various domains that are not contiguous in the native GP286 protein.

The invention also provides polypeptides comprising select portions of GP286 and related proteins. As will be further discussed herein below, these protein fragments, especially when coupled to heterologous protein fragments, can be used, for example, to target agents to particular cell types through protein-protein interaction; to inhibit protein-protein interactions between Ig domain containing proteins; for competitive binding assays; and to raise fragment-specific GP286 antibodies.

In some embodiments, the protein fragment comprises, in at least one copy, the Ig domain characteristic of the N-terminal extracellular portion of GP286. Specifically, the extracellular Ig domain is encoded by amino acids 32-110 of the GP286 amino acid sequence of SEQ ID NO:2 (see FIG. 1) and amino acids 37-115 of the GP286a amino acid sequence of SEQ ID NO: 15 (see FIG. 2).

Preferably, the protein fragment contains an N-terminal signal secretion sequence that will mediate transport of the polypeptide through a membrane. The GP286 signal secretion sequence is encoded by amino acids 1-17 of the GP286 amino acid sequence of SEQ ID NO:2 (see FIG. 1) and by amino acids 1-22 of the GP286a amino acid sequence of SEQ ID NO: 15 (see FIG. 2). Although the signal sequence may be from a heterologous protein, preferably the signal secretion sequence of the protein fragment is from GP286.

The above preferred protein fragments may optionally include a transmembrane domain, if insertion of the polypeptide into a membrane is so-desired. The transmembrane domain may be a GP286 domain (see below) or may be encoded by a heterologous gene encoding a transmembrane domain of a heterologous membrane-associated protein.

If so desired, the above preferred protein fragments may further comprise an intracellular C-terminal domain if specific signaling reactions are desired in response to GP286 binding interactions. The intracellular domain may be derived from GP286 or GP286a (see below) or may be encoded by a heterologous gene encoding an intracellular domain of a heterologous membrane-associated protein.

Other preferred embodiments of the protein fragments of the invention are those that comprise the transmembrane domain of GP286. Specifically, the GP286 transmembrane domain is encoded by amino acids 140-160 of the GP286 amino acid sequence of SEQ ID NO:2 (see FIG. 1) and by amino acids 145-165 of the GP286a amino acid sequence of SEQ ID NO: 15 (see FIG. 2).

Yet other preferred embodiments of the above-described protein fragments have a (C-terminal) intracellular domain of GP286. Specifically, one intracellular domain of GP286 is encoded by amino acids 161-186 of the GP286 amino acid sequence of SEQ ID NO:2 (see FIG. 1) and by amino acids 166-278 of the GP286a amino acid sequence of SEQ ID NO:15 (see FIG. 2). It is believed that these different intracellular domain forms are produced by alternative splicing.

As described above, the invention further provides proteins that differ in sequence from those described with particularity in the above-referenced sequence identifiers (SEQ ID NOs:), whether by way of insertion or deletion, by way of conservative or moderately conservative substitutions, as hybridization related proteins, or as cross-hybridizing proteins, with those that substantially retain a GP286 activity preferred. As also discussed above, the invention further provides fusions of the polypeptides, proteins and protein fragments herein described to heterologous polypeptides.

When used as immunogens, the various protein embodiments of the present invention can be used, inter alia, to elicit antibodies that bind to a variety of epitopes of the GP286 protein.

Other Defining Characteristics of GP286 Proteins

FIGS. 1 and 2 present the predicted amino acid sequences encoded by the gp286 cDNA clone (SEQ ID NO:2) and the gp286a cDNA clone (SEQ ID NO:14). Unless otherwise indicated, amino acid sequences of the proteins of the present invention were determined as a predicted translation from a nucleic acid sequence. Accordingly, any amino acid sequence presented herein may contain errors due to errors in the nucleic acid sequence, as described in detail above. Furthermore, single nucleotide polymorphisms (SNPs) occur frequently in eukaryotic genomes—more than 1.4 million SNPs have already identified in the human genome, International Human Genome Sequencing Consortium, Nature 409:860-921 (2001)—and the sequence determined from one individual of a species may differ from other allelic forms present within the population. Small deletions and insertions can often be found that do not alter the function of the protein.

Accordingly, the present invention provides GP286 polypeptides not only identical in sequence to those described with particularity herein, but also isolated proteins at least about 80% identical in sequence to those described with particularity herein, typically at least about 85%, 90%, 91%, 92%, 93%, 94%, or 95% identical in sequence to those described with particularity herein, usefully at least about 96%, 97%, 98%, or 99% identical in sequence to those described with particularity herein, and, most conservatively, at least about 99.5%, 99.6%, 99.7%, 99.8% and 99.9% identical in sequence to those described with particularity herein. These sequence variants can be naturally occurring or can result from human intervention by way of random or directed mutagenesis.

For purposes herein, percent identity of two amino acid sequences is determined using the procedure of Tatiana et al., “Blast 2 sequences—a new tool for comparing protein and nucleotide sequences”, FEMS Microbiol Lett. 174:247-250 (1999), which procedure is effectuated by the computer program Blast 2 SEQUENCES, available online at:

http://www.ncbi.nllm.nih.gov/Blast/bl2seq/bl2.html,

To assess percent identity of amino acid sequences, the BlastP module of Blast 2 SEQUENCES is used with default values of (i) BLOSUM62 matrix, Henikoff et al, Proc. Natl. Acad Sci USA 89(22):10915-9 (1992); (ii) open gap 11 and extension gap 1 penalties; and (iii) gap x_dropoff 50 expect 10 word size 3 filter, and both sequences are entered in their entireties.

As is well known, amino acid substitutions occur frequently among natural allelic variants, with conservative substitutions often occasioning only de minimis change in protein function. Accordingly, the present invention provides proteins not only identical in sequence to those described with particularity herein, but also isolated proteins having the sequence of GP286 proteins, or portions thereof, with conservative amino acid substitutions. Also provided are isolated proteins having the sequence of GP286 proteins, and portions thereof with moderately conservative amino acid substitutions. These conservatively-substituted or moderately conservatively-substituted variants can be naturally occurring or can result from human intervention.

As is also well known in the art, relatedness of proteins can also be characterized using a functional test, the ability of the encoding nucleic acids to base-pair to one another at defined hybridization stringencies. It is, therefore, another aspect of the invention to provide isolated proteins not only identical in sequence to those described with particularity herein, but also to provide isolated proteins (“hybridization related proteins”) that are encoded by nucleic acids that hybridize under high stringency conditions (as defined herein above) to all or to a portion of various of the isolated polynucleotides of the present invention (“reference nucleic acids”).

The hybridization related proteins can be alternative isoforms, homologs, paralogs, and orthologs of the GP286 protein of the present invention. Particularly useful orthologs are those from other primate species, such as chimpanzee, rhesus macaque monkey, baboon, orangutan, and gorilla; from rodents, such as rats, mice, guinea pigs; from lagomorphs, such as rabbits, and from domestic livestock, such as cow, pig, sheep, horse, goat.

Relatedness of proteins can also be characterized using a second functional test, the ability of a first protein to inhibit competitively the binding of a second protein to an antibody. It is, therefore, another aspect of the present invention to provide isolated proteins not only identical in sequence to those described with particularity herein, but also to provide isolated proteins (“cross-reactive proteins”) that competitively inhibit the binding of antibodies to all or to a portion of various of the isolated GP286 proteins of the present invention (“reference proteins”). Such competitive inhibition can readily be determined using immunoassays well known in the art.

Among the proteins of the present invention that differ in amino acid sequence from those described with particularity herein—including those that have deletions and insertions causing up to 10% non-identity, those having conservative or moderately conservative substitutions, hybridization related proteins, and cross-reactive proteins—those that substantially retain one or more GP286 activities are preferred (see supra).

Residues that are tolerant of change while retaining function can be identified by altering the protein at known residues using methods known in the art, such as alanine scanning mutagenesis, Cunningham et al., Science 244(4908): 1081-5 (1989); transposon linker scanning mutagenesis, Chen et al., Gene 263(1-2):39-48 (2001); combinations of homolog- and alanine-scanning mutagenesis, Jin et al., J. Mol. Biol. 226(3):851-65 (1992); combinatorial alanine scanning, Weiss et al., Proc. Natl. Acad. Sci USA 97(16):89504 (2000), followed by functional assay. Transposon linker scanning kits are available commercially (New England Biolabs, Beverly, Mass., USA, catalog. no. E7-102S; EZ::TN™ In-Frame Linker Insertion Kit, catalogue no. EZI04KN, Epicentre Technologies Corporation, Madison, Wis. USA).

As further described below, the isolated proteins of the present invention can readily be used as specific immunogens to raise antibodies that specifically recognize GP286 proteins, their isoforms, homologs, paralogs, and/or orthologs. The antibodies, in turn, can be used, inter alia, specifically to assay for the GP286 proteins of the present invention—e.g. by ELISA for detection of protein fluid samples, such as serum, by immunohistochemistry or laser scanning cytometry, for detection of protein in tissue samples, or by flow cytometry, for detection of intracellular protein in cell suspensions—for specific antibody-mediated isolation and/or purification of GP286 proteins, as for example by immunoprecipitation, and for use as specific agonists or antagonists of GP286 action. Further, the antibodies can be used to identify human T cells, which is useful in medical applications and in forensic science. The antibodies may also be used to isolate human T cells, e.g., by FACS, which is useful for treating T cells in vitro and for subsequent reinfusion into a patient. The antibodies may be conjugated to toxins or may be engineered to form fusion proteins comprising a toxin, which can be used to mark and kill aberrant T cells in a patient.

The isolated proteins of the present invention are also immediately available for use as specific standards in assays used to determine the concentration and/or amount specifically of the GP286 proteins of the present invention. As is well known, ELISA kits for detection and quantitation of protein analytes typically include isolated and purified protein of known concentration for use as a measurement standard (e.g., the human interferon-γ OptEIA kit, catalog no. 555142, Pharmingen, San Diego, Calif., USA includes human recombinant gamma interferon, baculovirus produced). The isolated proteins of the present invention are also immediately available for use as specific biomolecule capture probes for surface-enhanced laser desorption ionization (SELDI) detection of protein-protein interactions, WO 98/59362; WO 98/59360; WO 98/59361; and Merchant et al, Electrophoresis 21(6):1164-77 (2000), the disclosures of which are incorporated herein by reference in their entireties. Analogously, the isolated proteins of the present invention are also immediately available for use as specific biomolecule capture probes on BIACORE surface plasmon resonance probes. See Weinberger et al., Pharmacogenomics 1(4):395416 (2000); Malmqvist, Biochem. Soc. Trans. 27(2):33540 (1999).

The polypeptides of the instant invention may be conjugated to a toxin, or engineered to form a fusion protein with a toxin. The GP286-toxin may be used to bind to and kill GP286-binding cells. The polypeptides of the instant invention may also be conjugated to a detectable label or engineered to form a fusion protein with a detectable label. The GP286-label may be used to bind to and detect GP286-binding cells.

The isolated proteins of the present invention are also useful as a therapeutic supplement in patients diagnosed to have a specific deficiency in GP286 production or activity. Alternatively, the extracellular domain, or the portion thereof that is active in binding to another cell or to an extracellular matrix protein, may be administered to a patient to compete for binding of the cell surface GP286 to the other cell or extracellular matrix protein. Preferably, the extracellular domain or portion thereof is part of a fusion protein that increases serum half-life, e.g., all or a portion of an IgG heavy chain (see below).

The invention also provides fragments of various of the proteins of the present invention. The protein fragments are useful as antigenic and immunogenic fragments of GP286. By “fragments” of a protein is here intended isolated proteins (equally, polypeptides, peptides, oligopeptides), however obtained, that have an amino acid sequence identical to a portion of the reference amino acid sequence, which portion is at least 6 amino acids and less than the entirety of the reference nucleic acid. As so defined, “fragments” need not be obtained by physical fragmentation of the reference protein, although such provenance is not thereby precluded.

Fragments of at least 6 contiguous amino acids are useful in mapping B cell and T cell epitopes of the reference protein. See, e.g., Geysen et al., “Use of peptide synthesis to probe viral antigens for epitopes to a resolution of a single amino acid,” Proc. Natl. Acad. Sci. USA 81:39984002 (1984) and U.S. Pat. Nos. 4,708,871 and 5,595,915, the disclosures of which are incorporated herein by reference in their entireties. Because the fragment need not itself be immunogenic, part of an immunodominant epitope, nor even recognized by native antibody, to be useful in such epitope mapping, all fragments of at least 6 amino acids of the proteins of the present invention have utility in such a study.

Fragments of at least eight contiguous amino acids, often at least fifteen contiguous amino acids, have utility as immunogens for raising antibodies that recognize the proteins of the present invention. See, e.g., Lerner, “Tapping the immunological repertoire to produce antibodies of predetermined specificity,” Nature 299:592-596 (1982); Shinnick et al, “Synthetic peptide immunogens as vaccines,” Annu. Rev. Microbiol. 37:42546 (1983); Sutcliffe et al., “Antibodies that react with predetermined sites on proteins,” Science 219:660-6 (1983), the disclosures of which are incorporated herein by reference in their entireties. As further described in the above-cited references, virtually all 8-mers, conjugated to a carrier, such as a protein, prove immunogenic—that is, prove capable of eliciting antibody for the conjugated peptide; accordingly, all fragments of at least 8 amino acids of the proteins of the present invention have utility as immunogens.

Fragments of at least 8, 9, 10 or 12 contiguous amino acids are also useful as competitive inhibitors of binding of the entire protein, or a portion thereof, to antibodies (as in epitope mapping), and to natural binding partners, such as subunits in a multimeric complex or to receptors or ligands of the subject protein; this competitive inhibition permits identification and separation of molecules that bind-specifically to the protein of interest, U.S. Pat. Nos. 5,539,084 and 5,783,674, incorporated herein by reference in their entireties.

The protein, or protein fragment, of the present invention is thus at least 6 amino acids in length, typically at least 8, 9, 10 or 12 amino acids in length, and often at least 15 amino acids in length. Often, the protein or the present invention, or fragment thereof, is at least 20, 25, 30, 35, or 50 amino acids or more in length. Larger fragments having at least 75, 100, 150 or more amino acids are also useful, and at times preferred.

The present invention further provides fusions of each of the GP286 proteins and protein fragments of the present invention to heterologous polypeptides. By fusion is here intended that the protein or protein fragment of the present invention is linearly contiguous to the heterologous polypeptide in a peptide-bonded polymer of amino acids or amino acid analogues; by “heterologous polypeptide” is here intended a polypeptide that does not naturally occur in contiguity with the protein or protein fragment of the present invention. As so defined, the fusion can consist entirely of a plurality of fragments of the GP286 protein in altered arrangement; in such case, any of the GP286 fragments can be considered heterologous to the other GP286 fragments in the fusion protein. More typically, however, the heterologous polypeptide is not drawn from the GP286 protein itself.

The fusion proteins of the present invention will include at least one fragment of the protein of the present invention, which fragment is at least 6, typically at least 8, often at least 15, and usefully at least 16, 17, 18, 19, or 20 amino acids long. The fragment of the protein of the present to be included in the fusion can usefully be at least 25, 50, 75, 100, or 150 amino acids long. Fusions that include the entirety of the proteins of the present invention have particular utility.

The heterologous polypeptide included within the fusion protein of the present invention is at least 6 amino acids in length, often at least 8 amino acids in length, and preferably, at least 15, 20, and 25 amino acids in length. Fusions that include larger polypeptides, such as the IgG Fc region, and even entire proteins (such as luciferase or GFP chromophore-containing proteins), have particular utility.

As described above in the description of vectors and expression vectors of the present invention, heterologous polypeptides included in the fusion proteins of the present invention usefully include those designed to facilitate purification and/or visualization of recombinantly-expressed proteins. Although purification tags can also be incorporated into fusions that are chemically synthesized, chemical synthesis typically provides sufficient purity that further purification by HPLC suffices; however, visualization tags as above described retain their utility even when the protein is produced by chemical synthesis, and when so included render the fusion proteins of the present invention useful as directly detectable markers of GP286 presence.

As also discussed above, heterologous polypeptides to be included in the fusion proteins of the present invention can usefully include those that facilitate secretion of recombinantly expressed proteins—into the periplasmic space or extracellular milieu for prokaryotic hosts, into the culture medium for eukaryotic cells—through incorporation of secretion signals and/or leader sequences.

Other useful protein fusions of the present invention include those that permit use of the protein of the present invention as bait in a yeast two-hybrid system. See Bartel et al. (eds.), The Yeast Two-Hybrid System, Oxford University Press (1997) (ISBN: 0195109384); Zhu et al, Yeast Hybrid Technologies, Eaton Publishing, (2000) (ISBN 1-881299-15-5); Fields et al, Trends Genet. 10(8):286-92 (1994); Mendelsohn et al, Curr. Opin. Biotechnol. 5(5):482-6 (1994); Luban et al, Curr. Opin. Biotechnol. 6(1):59-64 (1995); Allen et al., Trends Biochem. Sci. 20(12):511-6 (1995); Drees, Curr. Opin. Chem. Biol. 3(1):64-70 (1999); Topcu et al, Pharm. Res. 17(9):1049-55 (2000); Fashena et al., Gene 250(1-2):1-14 (2000), the disclosures of which are incorporated herein by reference in their entireties. Typically, such fusion is to either E. coli LexA or yeast GALA DNA binding domains. Related bait plasmids are available that express the bait fused to a nuclear localization signal.

Other useful protein fusions include those that permit display of the encoded protein on the surface of a phage or cell, fusions to intrinsically delectable proteins, such as fluorescent or light-emitting proteins, and fusions to stable protein domains such as an immunoglobulin heavy chain domain like the IgG Fc region, as described above.

The proteins and protein fragments of the present invention can also usefully be fused to protein toxins, such as Pseudomonas exotoxin A, diphtheria toxin, shiga toxin A, anthrax toxin lethal factor, ricin, or other biologically deleterious moieties in order to effect specific ablation of cells that bind or take up the proteins of the present invention.

The isolated proteins, protein fragments, and protein fusions of the present invention can be composed of natural amino acids linked by native peptide bonds, or can contain any or all of nonnatural amino acid analogues, normative bonds, and post-synthetic (post translational) modifications, either throughout the length of the protein or localized to one or more portions thereof.

As is well known in the art, when the isolated protein is used, e.g., for epitope mapping, the range of such nonnatural analogues, normative inter-residue bonds, or post-synthesis modifications will be limited to those that permit binding of the peptide to antibodies. When used as an immunogen for the preparation of antibodies in a non-human host, such as a mouse, the range of such nonnatural analogues, normative inter-residue bonds, or post-synthesis modifications will be limited to those that do not interfere with the immunogenicity of the protein. When the isolated protein is used as a therapeutic agent, such as a vaccine or for replacement therapy, the range of such changes will be limited to those that do not confer toxicity upon the isolated protein.

Techniques for incorporating non-natural amino acids during solid phase chemical synthesis or by recombinant methods are well established in the art. Procedures are described, inter alia, in Chan et al. (eds.), Fmoc Solid Phase Peptide Synthesis: A Practical Approach (Practical Approach Series), Oxford Univ. Press (March 2000) (ISBN: 0199637245); Jones, Amino Acid and Peptide Synthesis (Oxford Chemistry Primers, No 7), Oxford Univ. Press (August 1992) (ISBN: 0198556683); and Bodanszky, Principles of Peptide Synthesis (Springer Laboratory), Springer Verlag (December 1993) (ISBN: 0387564314), the disclosures of which are incorporated herein by reference in their entireties.

D-enantiomers of natural amino acids can readily be incorporated during chemical peptide synthesis: peptides assembled from D-amino acids are more resistant to proteolytic attack; incorporation of D-enantiomers can also be used to confer specific three dimensional conformations on the peptide. Other amino acid analogues commonly added during chemical synthesis include ornithine, norleucine, phosphorylated amino acids (typically phosphoserine, phosphothreonine, phosphotyrosine), L-malonyltyrosine, a non-hydrolyzable analog of phosphotyrosine (Kole et al., Biochem. Biophys. Res. Com. 209:817-821 (1995)), and various halogenated phenylalanine derivatives.

Amino acid analogues having detectable labels are also usefully incorporated during synthesis to provide a labeled polypeptide. Biotin, for example can be added using biotinoyl—(9-fluorenylmethoxycarbonyl)-L-lysine (FMOC biocytin) (Molecular Probes, Eugene, Oreg., USA). (Biotin can also be added enzymatically by incorporation into a fusion protein of a E. coli BirA substrate peptide.) The FMOC and tBOC derivatives of dabcyl-L-lysine (Molecular Probes, Inc., Eugene, Oreg., USA) can be used to incorporate the dabcyl chromophore at selected sites in the peptide sequence during synthesis. The aminonaphthalene derivative EDANS, the most common fluorophore for pairing with the dabcyl quencher in fluorescence resonance energy transfer (FRET) systems, can be introduced during automated synthesis of peptides by using EDANS—FMOC-L-glutamic acid or the corresponding tBOC derivative (both from Molecular Probes, Inc., Eugene, Oreg., USA). Tetramethylrhodamine fluorophores can be incorporated during automated FMOC synthesis of peptides using (FMOC)-TMR-L-lysine (Molecular Probes, Inc. Eugene, Oreg., USA).

Other useful amino acid analogues that can be incorporated during chemical synthesis include aspartic acid, glutamic acid, lysine, and tyrosine analogues having allyl side-chain protection (Applied Biosystems, Inc., Foster City, Calif., USA); the allyl side chain permits synthesis of cyclic, branched-chain, sulfonated, glycosylated, and phosphorylated peptides.

A large number of other FMOC-protected non-natural amino acid analogues capable of incorporation during chemical synthesis are available commercially, e.g., from The Peptide Laboratory (Richmond, Calif., USA).

Non-natural residues can also be added biosynthetically by engineering a suppressor tRNA, typically one that recognizes the UAG stop codon, by chemical aminoacylation with the desired unnatural amino acid and. Conventional site-directed mutagenesis is used to introduce the chosen stop codon UAG at the site of interest in the protein gene. When the acylated suppressor tRNA and the mutant gene are combined in an in vitro transcription/translation system, the unnatural amino acid is incorporated in response to the UAG codon to give a protein containing that amino acid at the specified position. Liu et al., Proc. Natl. Acad. Sci. USA 96(9):4780-5 (1999); Wang et al., Science 292(5516):498-500 (2001).

The isolated GP286 proteins, protein fragments and fusion proteins of the present invention can also include non-native inter-residue bonds, including bonds that lead to circular and branched forms. The isolated GP286 proteins and protein fragments of the present invention can also include post-translational and post-synthetic modifications, either throughout the length of the protein or localized to one or more portions thereof.

For example, when produced by recombinant expression in eukaryotic cells, the isolated proteins, fragments, and fusion proteins of the present invention will typically include N-linked and/or O-linked glycosylation, the pattern of which will reflect both the availability of glycosylation sites on the protein sequence and the identity of the host cell. Further modification of glycosylation pattern can be performed enzymatically. As another example, recombinant polypeptides of the invention may also include an initial modified methionine residue, in some cases resulting from host-mediated processes.

When the proteins, protein fragments, and protein fusions of the present invention are produced by chemical synthesis, post-synthetic modification can be performed before deprotection and cleavage from the resin or after deprotection and cleavage. Modification before deprotection and cleavage of the synthesized protein often allows greater control, e.g. by allowing targeting of the modifying moiety to the N-terminus of a resin-bound synthetic peptide. Useful post-synthetic (and post-translational) modifications include conjugation to detectable labels, such as fluorophores. A wide variety of amine-reactive and thiol-reactive fluorophore derivatives have been synthesized that react under nondenaturing conditions with N-terminal amino groups and epsilon amino groups of lysine residues, on the one hand, and with free thiol groups of cysteine residues, on the other.

Kits are available commercially that permit conjugation of proteins to a variety of amine-reactive or thiol-reactive fluorophores: Molecular Probes, Inc. (Eugene, Oreg., USA), e.g., offers kits for conjugating proteins to Alexa Fluor 350, Alexa Fluor 430, Fluorescein-EX, Alexa Fluor 488, Oregon Green 488, Alexa Fluor 532, Alexa Fluor 546, Alexa Fluor 546, Alexa Fluor 568, Alexa Fluor 594, and Texas Red-X. A wide variety of other amine-reactive and thiol-reactive fluorophores are available commercially (Molecular Probes, Inc., Eugene, Oreg., USA), including Alexa Fluor® 350, Alexa Fluor® 488, Alexa Fluor® 532, Alexa Fluor® 546, Alexa Fluor® 568, Alexa Fluor® 594, Alexa Fluor® 647 (monoclonal antibody labeling kits), BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red.

The polypeptides of the present invention can also be conjugated to fluorophores, other proteins, and other macromolecules, using bifunctional linking reagents. Common homobifunctional reagents include, e.g., APG, AEDP, BASED, BMB, BMDB, BMH BMOE, BM[PEO]3, BM[PEO]4, BS3, BSOCOES, DFDNB, DMA, DMP, DMS, DPDPB, DSG, DSP (Lomant's Reagent), DSS, DST, DTBP, DTME, DTSSP, EGS, HBVS, Sulfo-BSOCOES, Sulfo-DST, Sulfo-EGS (all available from Pierce, Rockford, Ill., USA); common heterobifunctional cross-linkers include ABH AMAS, ANB-NOS, APDP, ASBA, BMPA, BMPH, BMPS, EDC, EMCA, EMCH, EMCS, KMUA, KMUH GMBS, LC-SMCC, LC-SPDP, MBS, M2C2H, MPBH, MSA, NHS-ASA, PDPH, PMPI, SADP, SAED, SAND, SANPAH, SASD, SATP, SBAP, SFAD, SIA, SIAB, SMCC, SMPB, SMPH, SMPT, SPDP, Sulfo-EMCS, Sulfo-GMBS, Sulfo-HSAB, Sulfo-KMUS, Sulfo-LC-SPDP, Sulfo-MBS, Sulfo-NHS-LC-ASA, Sulfo-SADP, Sulfo-SANPAH, Sulfo-SIAB, Sulfo-SMCC, Sulfo-SMPB, Sulfo-LC-SMPT, SVSB, TFCS (all available Pierce, Rockford, Ill., USA).

The proteins, protein fragments, and protein fusions of the present invention can be conjugated, using such cross-linking reagents, to fluorophores that are not amine- or thiol-reactive. Other labels that usefully can be conjugated to the proteins, protein fragments, and fusion proteins of the present invention include radioactive labels, echosonographic contrast reagents, and MRI contrast agents. The proteins, protein fragments, and protein fusions of the present invention can also usefully be conjugated using cross-linking agents to carrier proteins, such as KLH, bovine thyroglobulin, and even bovine serum albumin (BSA), to increase iimrnunogenicity for raising anti-GP286 antibodies.

The GP286 proteins, protein fragments, and protein fusions of the present invention can also usefully be conjugated to polyethylene glycol (PEG); PEGylation increases the serum half life of proteins administered intravenously for replacement therapy. Delgado et al., Crit. Rev. Ther. Drug Carrer Syst. 9(3-4):249-304 (1992); Scott et al., Curr. Pharm. Des. 4(6):423-38 (1998); DeSantis et al., Curr. Opin. Biotechnol. 10(4):324-30 (1999), incorporated herein by reference in their entireties. PEG monomers can be attached to the protein directly or through a linker, with PEGylation using PEG monomers activated with tresyl chloride (2,2,2-trifluoroethanesulphonyl chloride) permitting direct attachment under mild conditions.

The isolated GP286 proteins of the present invention, including fusions thereof can be produced by recombinant expression, typically using the expression vectors of the present invention as above-described or, especially if fewer than about 100 amino acids, optionally by chemical synthesis (typically, solid phase synthesis), and, on occasion, by in vitro translation.

Production of the isolated proteins of the present invention can optionally be followed by purification. Purification of recombinantly expressed proteins is now well within the skill in the art. See, e.g., Thorner et al. (eds.), Applications of Chimeric Genes and Hybrid Proteins, Part A: Gene Expression and Protein Purification (Methods in Enzymology, Volume 326), Academic Press (2000), (ISBN: 0121822273); Harbin (ed.), Cloning, Gene Expression and Protein Purification: Experimental Procedures and Process Rationale, Oxford Univ. Press (2001) (ISBN: 0195132947); Marshak et al., Strategies for Protein Purification and Characterization: A Laboratory Course Manual, Cold Spring Harbor Laboratory Press (1996) (ISBN: 0-87969-385-1); and Roe (ed.), Protein Purification Applications, Oxford University Press (2001), the disclosures of which are incorporated herein by reference in their entireties, and thus need not be detailed here.

Briefly, however, if purification tags have been fused through use of an expression vector that appends such tag, purification can be effected, at least in part, by means appropriate to the tag, such as use of immobilized metal affinity chromatography for polyhistidine tags. Other techniques common in the art include ammonium sulfilte fractionation, immuno-precipitation, fast protein liquid chromatography (FPLC), high performance liquid chromatography (HPLC), and preparative gel electrophoresis. Purification of chemically-synthesized peptides can readily be effected, e.g., by HPLC.

Accordingly, it is an aspect of the present invention to provide the isolated GP286 proteins of the present invention in pure or substantially pure form. A purified protein of the present invention is an isolated protein, as above described, that is present at a concentration of at least 95%, as measured on a mass basis (w/w) with respect to total protein in a composition. Such purities can often be obtained during chemical synthesis without further purification, as, e.g., by HPLC. Purified proteins of the present invention can be present at a concentration (measured on a mass basis with respect to total protein in a composition) of 96%, 97%, 98%, and even 99%. The proteins of the present invention can even be present at levels of 99.5%, 99.6%, and even 99.7%, 99.8%, or even 99.9% following purification, as by HPLC.

Although high levels of purity are preferred when the isolated proteins of the present invention are used as therapeutic agents—such as vaccines, or for replacement therapy—the isolated proteins of the present invention are also useful at lower purity. For example, partially purified proteins of the present invention can be used as immunogens to raise antibodies in laboratory animals.

Thus, the present invention provides the isolated proteins of the present invention in substantially purified form. A “substantially purified protein” of the present invention is an isolated protein, as above described, present at a concentration of at least 70%, measured on a mass basis with respect to total protein in a composition. Usefully, the substantially purified protein is present at a concentration, measured on a mass basis with respect to total protein in a composition, of at least 75%, 80%, or even at least 85%, 90%, 91%, 92%, 93%, 94%, 94.5% or even at least 94.9%.

In preferred embodiments, the purified and substantially purified proteins of the present invention are in compositions that lack detectable ampholytes, acrylamide monomers, bis-acrylamide monomers, and polyacrylamide.

The GP286 proteins, fragments, and fusions of the present invention can usefully be attached to a substrate. The substrate can porous, substantially nonporous (such as plastic), or solid; planar or non-planar; the bond can be covalent or noncovalent. Porous substrates, commonly membranes, typically comprise nitrocellulose, polyvinylidene fluoride (PVDF), or cationically derivatized, hydrophilic PVDF; so bound, the proteins, fragments, and fusions of the present invention can be used to detect and quantify antibodies, e.g. in serum, that bind specifically to the immobilized protein of the present invention. Proteins, fragments, and fusions of the present invention when bound to substantially nonporous substrates, such as plastics, may be used to detect and quantify antibodies, e.g. in serum, that bind specifically to the immobilized protein of the present invention.

The proteins, fragments, and fusions of the present invention can also be attached to a substrate suitable for use as a surface enhanced laser desorption ionization source; so attached, the protein, fragment, or fusion of the present invention is useful for binding and then detecting secondary proteins that bind with sufficient affinity or avidity to the surface-bound protein to indicate biologic interaction therebetween.

The proteins, fragments, and fusions of the present invention can also be attached to a substrate suitable for use in surface plasmon resonance detection. So attached, the protein, fragment, or fusion of the present invention is useful for binding and then detecting secondary proteins that bind with sufficient affinity or avidity to the surface-bound protein to indicate significant biological interaction between the two.

Antibodies and Antibody-Producing Cells

The invention provides antibodies, including fragments and derivatives thereof that bind specifically to GP286 proteins and protein fragments of the invention, or that bind to one or more of the proteins and protein fragments encoded by the isolated GP286 nucleic acids of the invention. The antibodies can be specific for linear epitopes, discontinuous epitopes, or conformational epitopes of such proteins or protein fragments, either as present on the protein in its native conformation or, in some cases, as present on the proteins as denatured, as, e.g., by solubilization in SDS.

The invention also provides antibodies, including fragments and derivatives thereof the binding of which can be competitively inhibited by one or more of the GP286 proteins and protein fragments of the present invention, or by one or more of the proteins and protein fragments encoded by the isolated gp286 polynucleotides of the present invention.

In a first series of antibody embodiments, the invention provides antibodies, both polyclonal and monoclonal, and fragments and derivatives thereof, that bind specifically to a polypeptide having an amino acid sequence in SEQ ID NO:2, i.e., a full length gp286 protein.

Such antibodies are useful in a variety of in vitro immunoassays, such as Western blotting and ELISA Such antibodies are also useful in isolating and purifying GP286 proteins, including related cross-reactive proteins, by immuno-precipitation, immunoaffinity chromatography, or magnetic bead-mediated purification. Such methods are well-known in the art. Anti-GP286 antibodies are also useful for identifying T cells, marking and killing T cells and detectably labeling T cells (see above).

In a second series of antibody embodiments, the invention provides antibodies, both polyclonal and monoclonal, and fragments and derivatives thereof, the specific binding of which can be competitively inhibited by the isolated proteins and polypeptides of the present invention.

In other embodiments, the invention further provides the above-described antibodies detectably labeled, and in yet other embodiments, provides the above-described antibodies attached to a substrate.

As used herein, the term “antibody” refers to a polypeptide, at least a portion of which is encoded by at least one immunoglobulin gene, which can bind specifically to a first molecular species, and to fragments or derivatives thereof that remain capable of such specific binding.

By “bind specifically” and “specific binding” is here intended the ability of the antibody to bind to a first molecular species in preference to binding to other molecular species with which the antibody and first molecular species are admixed. An antibody is said specifically to “recognize” a first molecular species when it can bind specifically to that first molecular species.

As is well known in the art, the degree to which an antibody can discriminate as among molecular species in a mixture will depend, in part, upon the conformational relatedness of the species in the mixture; typically, the antibodies of the present invention will discriminate over adventitious binding to non-GP286 proteins by at least two-fold, more typically by at least 5-fold, typically by more than 10-fold, 25-fold, 50-fold, 75-fold, and often by more than 100-fold, and on occasion by more than 500-fold or 1000-fold. When used to detect the proteins or protein fragments of the present invention, the antibody of the present invention is sufficiently specific when it can be used to determine the presence of the protein of the present invention in samples derived from human pancreatic and neural tissues.

Typically, the affinity or avidity of an antibody (or antibody multimer, as in the case of an IgM pentamer) of the present invention for a GP286 protein or protein fragment of the present invention will be at least about 1×10⁻⁶ molar (M), typically at least about 5×10⁻⁷ M, usefully at least about 1×10⁻⁷ M, with affinities and avidities of at least 1×10⁻⁸ M, 5×10⁻⁹ M and 1×10⁻¹⁰ M proving especially useful.

The antibodies of the present invention can be naturally-occurring forms, such as IgG, IgM, IgD, IgE, and IgA, from any mammalian species.

Human antibodies can, but will infrequently, be drawn directly from human donors or human cells. In such case, antibodies to the proteins of the present invention will typically have resulted from fortuitous immunization, such as autoimmune immunization, with the protein or protein fragments of the present invention. Such antibodies will typically, but will not invariably, be polyclonal.

Human antibodies are more frequently obtained using transgenic animals that express human immunoglobulin genes, which transgenic animals can be affirmatively immunized with a GP286 protein immunogen of the present invention. Human Ig-transgenic mice capable of producing human antibodies and methods of producing human antibodies therefrom upon specific immunization are well known in the art. See, e.g., in U.S. Pat. Nos. 6,162,963; 6,150,584; 6,114,598; 6,075,181; 5,939,598; 5,877,397; 5,874,299; 5,814,318; 5,789,650; 5,770,429; 5,661,016; 5,633,425; 5,625,126; 5,569,825; 5,545,807; 5,545,806, and 5,591,669, the disclosures of which are incorporated herein by reference in their entireties. Such antibodies are typically monoclonal, and are typically produced using techniques developed for production of murine antibodies.

Human antibodies are particularly useful, and often preferred, when the antibodies of the present invention are to be administered to human beings as in vivo diagnostic or therapeutic agents, since recipient immune response to the administered antibody will often be substantially less than that occasioned by administration of an antibody derived from another species, such as mouse.

IgG, IgM, IgD, IgE and IgA antibodies of the present invention are also usefully obtained from other mammalian species, including rodents—typically mouse, but also rat, guinea pig, and hamster—lagomorphs, typically rabbits, and also larger mammals, such as sheep, goats, cows, and horses. In such cases, as with the transgenic human-antibody-producing non-human mammals, fortuitous immunization is not required, and the non-human mammal is typically affirmatively immunized, according to standard immunization protocols, with the protein or protein fragment of the present invention.

As discussed above, virtually all fragments of eight or more contiguous amino acids of the proteins of the present invention can be used effectively as immunogens when conjugated to a carrier, typically a protein such as bovine thyroglobulin, keyhole limpet hemocyanin, or bovine serum albumin, conveniently using a bifunctional linker such as those described elsewhere above, which discussion is incorporated by reference here.

Immunogenicity can also be conferred by fusion of the proteins and protein fragments of the present invention to other moieties. Peptides of the present invention can, for example, be produced by solid phase synthesis on a branched polylysine core matrix; these multiple antigenic peptides (MAPs) provide high purity, increased avidity, accurate chemical definition and improved safety in vaccine development. Tam et al, Proc. Natl. Acad. Sci. USA 85:5409-5413 (1988); Posnett et al., J. Biol. Chem. 263, 1719-1725 (1988).

Protocols for immunizing non-human mammals are well-established in the art, Harlow et al. (eds.), Antibodies: A Laboratory Manual, 2d Ed., Cold Spring Harbor Laboratory (1999) (ISBN: 0879693142); Coligan et al (eds.), Current Protocols in Immunology, John Wiley & Sons, Inc. (2001) (ISBN: 0471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907), the disclosures of which are incorporated herein by reference.

Antibodies from nonhuman mammals can be polyclonal or monoclonal, with polyclonal antibodies having certain advantages in immuno-histochemical detection of the proteins of the present invention and monoclonal antibodies having advantages in identifying and distinguishing particular epitopes of the proteins of the present invention.

Following immunization, the antibodies of the present invention can be produced using any art-accepted technique. Such techniques are well known in the art, Coligan et al. (eds.), Current Protocols in Immunology, John Wiley & Sons, Inc. (2001) (ISBN: 0471-52276-7); Zola, Monoclonal Antibodies: Preparation and Use of Monoclonal Antibodies and Engineered Antibody Derivatives (Basics: From Background to Bench), Springer Verlag (2000) (ISBN: 0387915907); Howard et al (eds.), Basic Methods in Antibody Production and Characterization, CRC Press (2000) (ISBN: 0849394457); Harlow et al. (eds.), Antibodies: A Laboratory Manual, Cold Spring Harbor Laboratory (1998) (ISBN: 0879693142); Davis (ed.), Monoclonal Antibody Protocols, Vol. 45, Humana Press (1995) (ISBN: 0896033082); Delves (ed.), Antibody Production: Essential Technigues, John Wiley & Son Ltd (1997) (ISBN: 0471970107); Kenney, Antibody Solution: An Antibody Methods Manual, Chapman & Hall (1997) (ISBN: 0412141914), incorporated herein by reference in their entireties, and thus need not be detailed here.

Recombinant expression in host cells is particularly useful when fragments or derivatives of the antibodies of the present invention are desired. Host cells for recombinant antibody production—either whole antibodies, antibody fragments, or antibody derivatives—can be prokaryotic or eukaryotic.

Prokaryotic hosts are particularly useful for producing phage displayed antibodies of the present invention. The technology of phage-displayed antibodies, in which antibody variable region fragments are fused, for example, to the gene III protein (pIII) or gene VIII protein (pIII) for display on the surface of filamentous phage, such as M13, is by now well-established, Sidhu, Curr. Opin. Biotechnol. 11(6):610-6 (2000); Griffiths et al., Curr. Opin Biotechnol. 9(1):102-8 (1998); Hoogenboom et al., Immunotechnology, 4(1):1-20 (1998); Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433455 (1994), and techniques and protocols required to generate, propagate, screen (pan), and use the antibody fragments from such libraries have recently been compiled, Barbas et al., Phage Display: A Laboratory Manual, Cold Spring Harbor Laboratory Press (2001) (ISBN 0-87969-546-3); Kay et al. (eds.), Phage Display of Peptides and Proteins: A Laboratory Manual, Academic Press, Inc. (1996); Abelson et al. (eds.), Combinatorial Chemistry, Methods in Enzymology vol. 267, Academic Press (May 1996), the disclosures of which are incorporated herein by reference in their entireties. Typically, phage-displayed antibody fragments are scFv fragments or Fab fragments; when desired, full length antibodies can be produced by cloning the variable regions from the displaying phage into a complete antibody and expressing the full length antibody in a further prokaryotic or a eukaryotic host cell.

Eukaryotic cells are also useful for expression of the antibodies, antibody fragments, and antibody derivatives of the present invention. For example, antibody fragments of the present invention can be produced in Pichiapastoris, Takahashi et al., Biosci. Biotechnol. Biochem. 64(10):2138-44 (2000); Freyre et al., J. Biotechnol. 76(2-3):157-63 (2000); Fischer et al., Biotechnol. Appl. Biochem. 30 (Pt 2):117-20 (1999); Pennell et al., Res. Immunol. 149(6):599-603 (1998); Eldin et al., J. Immunol. Methods. 201(1):67-75 (1997); and in Saccharomyces cerevisiae, Frenken et al., Res. Immunol. 149(6):589-99 (1998); Shusta et al., Nature Biotechnol. 16(8):773-7 (1998), the disclosures of which are incorporated herein by reference in their entireties.

Antibodies, including antibody fragments and derivatives, of the invention can also be produced in insect cells, Li et al., Protein Expr. Purif. 21(1):121-8 (2001); Ailor et al., Biotechnol. Bioeng. 58(2-3):196-203 (1998); Hsu et al., Biotechnol. Prog. 13(1):96-104 (1997); Edelman et a., Immunology 91(1):13-9 (1997); and Nesbit et al., J. Immunol. Methods. 151(1-2):201-8 (1992), the disclosures of which are incorporated herein by reference in their entireties.

Antibodies and fragments and derivatives thereof of the present invention may also be produced in plant cells, Giddings et al., Nature Biotechnol. 18(11):1151-5 (2000); Gavilondo et al., Biotechniques 29(1):128-38 (2000); Fischer et al, J. Biol. Regul. Homeost. Agents 14(2):83-92 (2000); Fischer et al., Biotechnol. Appl. Biochem. 30 (Pt 2): 113-6 (1999); Fischer et al., Biol. Chem. 380(7-8):825-39 (1999); Russell, Curr. Top. Microbiol. Immunol. 240:119-38 (1999); and Ma et al., Plant Physiol. 109(2):341-6 (1995), the disclosures of which are incorporated herein by reference in their entireties.

Mammalian cells useful for recombinant expression of antibodies, antibody fragments, and antibody derivatives of the present invention include CHO cells, COS cells, 293 cells, and myeloma cells. Verma et a., J. Immunol. Methods 216(1-2):165-81 (1998), review and compare bacterial, yeast, insect and mammalian expression systems for expression of antibodies.

Antibodies of the present invention may also be prepared by cell free translation, as further described in Merk et al., J. Biochem. (Tokyo). 125(2):328-33 (1999) and Ryabova et al., Nature Biotechnol. 15(1):79-84 (1997), and in the milk of transgenic animals, as further described in Pollock et al., J. Immunol. Methods 231(1-2):147-57 (1999), the disclosures of which are incorporated herein by reference in their entireties.

The invention further provides antibody fragments that bind specifically to one or more of the GP286 proteins and protein fragments of the present invention, to one or more of the proteins and protein fragments encoded by the isolated gp286 polynucleotides of the present invention, or the binding of which can be competitively inhibited by one or more of the proteins and protein fragments of the present invention or one or more of the proteins and protein fragments encoded by the isolated polynucleotides of the present invention.

Among such useful fragments are Fab, Fab′, Fv, F(ab)′₂, and single chain Fv (scFv) fragments. Other useful fragments are described in Hudson, Curr. Opin. Biotechnol. 9(4):395-402 (1998). The present invention thus provides antibody derivatives that bind specifically to one or more of the GP286 proteins and protein fragments of the present invention, to one or more of the proteins and protein fragments encoded by the isolated nucleic acids of the present invention, or the binding of which can be competitively inhibited by one or more of the proteins and protein fragments of the present invention or one or more of the proteins and protein fragments encoded by the isolated polynucleotides of the present invention.

Among such useful derivatives are chimeric, primatized, and humanized antibodies; such derivatives are less immunogenic in human beings, and thus more suitable for in vivo administration, than are unmodified antibodies from non-human mammalian species.

Chimeric antibodies typically include heavy and/or light chain variable regions (including both CDR and framework residues) of immunoglobulins of one species, typically mouse, fused to constant regions of another species, typically human See, e.g., U.S. Pat. No. 5,807,715; Morrison et al., Proc. Natl. Acad Sci USA.81(21):6851-5 (1984); Sharon et al., Nature 309(5966):364-7 (1984); Takeda et al., Nature 314(6010):4524 (1985), the disclosures of which are incorporated herein by reference in their entireties.

Primatized and humanized antibodies typically include heavy and/or light chain CDRs from a murine antibody grafted into a non-human primate or human antibody V region framework, usually further comprising a human constant region, Riechmann et al, Nature 332(6162):323-7 (1988); Co et al., Nature 351(6326):501-2 (1991); U.S. Pat. Nos. 6,054,297; 5,821,337; 5,770,196; 5,766,886; 5,821,123; 5,869,619; 6,180,377; 6,013,256; 5,693,761; and 6,180,370, the disclosures of which are incorporated herein by reference in their entireties.

Other useful antibody derivatives of the invention include heteromeric antibody complexes and antibody fusions, such as diabodies (bispecific antibodies), single-chain diabodies, and intrabodies.

The antibodies of the present invention, including fragments and derivatives thereof, can usefully be labeled. It is, therefore, another aspect of the present invention to provide labeled antibodies that bind specifically to one or more of the proteins and protein fragments of the present invention, to one or more of the GP286 proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention.

The choice of label depends, in part, upon the desired use. When the antibodies of the present invention are used for immunohistochemical staining of tissue samples, the label can usefully be an enzyme that catalyzes production and local deposition of a detectable product. Enzymes typically conjugated to antibodies to permit their immunohistochemical visualization are well known, and include alkaline phosphatase, β-galacto-sidase, glucose oxidase, horseradish peroxidase (HRP), and urease. The antibodies of the invention can also be labeled using colloidal gold.

A multitude of typical substrates for production and deposition of visually detectable products, luminescent and fluorescent labels, are also well known and need not be further described. See, e.g., Thorpe et al., Methods Enzymol. 133:331-53 (1986); Kricka et al., J. Immunoassay 17(1):67-83 (1996); and Lundqvist et al., J. Biolumin. Chemilumin. 10(6):353-9 (1995), the disclosures of which are incorporated herein by reference in their entireties. Kits for enhanced chemiluminescent detection (ECL) are available commercially.

When the antibodies of the present invention are used, e.g., for flow cytometric detection, for fluorescent activated cell sorting (FACS), for scanning laser cytometric detection, or for fluorescent immunoassay, they can usefully be labeled with fluorophores. There are a wide variety of fluorophore labels that can usefully be attached to the antibodies of the present invention. Many are available, e.g., from Molecular Probes, Inc., Eugene, Oreg., USA.

For secondary detection using labeled avidin, streptavidin, captavidin or neutravidin, the antibodies of the present invention can usefully be labeled with biotin.

When the antibodies of the present invention are used, e.g. for Western blotting applications, they can usefully be labeled with radioisotopes, such as ³³P, ³²P, ³⁵S, ³H and ¹²⁵I. As another example, when the antibodies of the present invention are used for radioimmunotherapy, the label can usefully be ²²⁸Th, ²²⁷Ac, ²²⁵Ac, ²²³Ra, ²¹³Bi, ²¹²Pb, ²¹²Bi, ²¹¹At ²⁰³Pb, ¹⁹⁴Os, ¹⁸⁸Re, ¹⁸⁶Re, ¹⁵³Sm, ¹⁴⁹Tb, ¹³¹I, ¹²⁵I, ¹¹¹In, ¹⁰⁵Rh, ^{99m}Tc, ⁹⁷Ru, ⁹⁰Y, ⁹⁰Sr, ⁸⁸Y, ⁷²Se, ⁶⁷Cu, or ⁴⁷Sc. Radiolabeled anti-GP286 antibodies may be used to specifically target and kill T cells in a patient in need thereof. As another example, when the antibodies of the present invention are to be used for in vivo diagnostic use, they can be rendered detectable by conjugation to MRI contrast agents, such as gadolinium diethylenetriaminepentaacetic acid (DTPA), Lauffer et al., Radiology 207(2):529-38 (1998), or by radioisotopic labeling. As would be understood by the skilled artisan, use of any of the labels described above is not restricted to the application as for which they were mentioned.

The antibodies of the present invention, including fragments and derivatives thereof can also be conjugated to biologically deleterious moieties, such as toxins, in order to target the toxin's ablative action to cells that display and/or express the proteins of the present invention. As discussed above, toxin-conjugated anti-GP286 antibodies may be used to bind to and kill T cells in a patient in need thereof Commonly, the antibody in such immunotoxins is conjugated to Pseudomonas exotoxin A, diphtheria toxin, shiga toxin A, anthrax toxin lethal factor, or ricin. See Hall (ed.), Immunotoxin Methods and Protocols (Methods in Molecular Biology, Vol 166), Humana Press (2000) (ISBN:0896037754); and Frankel et al. (eds.), Clinical Applications of Immunotoxins, Springer-Verlag New York, Incorporated (1998) (ISBN:3540640975), the disclosures of which are incorporated herein by reference in their entireties, for review.

The antibodies of the present invention can usefully be attached to a substrate. The invention thus provides antibodies that bind specifically to one or more of the GP286 proteins and protein fragments of the present invention, to one or more of the proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention, attached to a substrate. Substrates can be porous or nonporous, planar or nonplanar.

For example, the antibodies of the present invention can usefully be conjugated to filtration media, such as NHS-activated Sepharose or CNBr-activated Sepharose for purposes of immunoaffinity chromatography.

The antibodies of the present invention can also usefully be attached to paramagnetic microspheres, typically by biotin-streptavidin interaction, which microsphere can then be used for isolation of cells that express or display the proteins of the present invention. As another example, the antibodies of the present invention can usefully be attached to the surface of a microtiter plate for ELISA

As noted above, the antibodies of the present invention can be produced in prokaryotic and eukaryotic cells. The invention thus also provides cells that express the antibodies of the present invention, including hybridoma cells, B cells, plasma cells, and host cells recombinantly modified to express the antibodies of the present invention.

The present invention also provides aptamers evolved to bind specifically to one or more of the GP286 proteins and protein fragments of the present invention, to one or more of the proteins and protein fragments encoded by the isolated polynucleotides of the present invention, or the binding of which can be competitively inhibited by one or more of the proteins and protein fragments of the present invention or one or more of the proteins and protein fragments encoded by the isolated polynucleotides of the present invention.

Pharmaceutical Compositions and Therapeutic Methods

GP286 is a new member of the immunoglobulin (Ig) superfamily expressed on T cells. Expression in other tissues and organs is likely due to T cell infiltration of other tissues. GP286, an integral cell surface membrane protein, has one signature Ig domain in its extracellular portion, which is known in other family members to mediate cell-cell recognition and adhesion reactions. As a member of the Ig superfamily, GP286 is likely important for mediating cell-cell recognition, binding and adhesion functions in the immune system, particularly in T cells.

The invention accordingly provides pharmaceutical compositions comprising nucleic acids, proteins, and antibodies of the present invention, as well as mimetics, agonists, antagonists, or modulators of GP286 activity, may be administered as pharmaceutical agents for the treatment (i.e., the amelioration of) of disorders, conditions or diseases associated with mis-expression of GP286 or to overcome abnormal expression or activities of other components which participate in GP286 related molecular and cellular recognition pathways. As GP286 expression is expressed in T cells, it is anticipated that GP286 mis-expression may be associated with immune disorders or diseases, and/or with congenital defects in immune development or function.

T-cell mediated diseases include, without limitation, transplantation disorders, autoimmune disease, cancer, multiple sclerosis, graft versus host disease and Kawasaki syndrome. Examples of autoimmune disease include rheumatoid arthritis, systemic lupus erythematosus, psoriasis, Sjogren's Syndrome, thyroiditis, Graves' disease, pulmonary fibrosis, bronchiolitis obliterans, hemolytic anemia and Wegener's granulomatosis. Examples of cancer include leukemia and lymphoma, including, without limitation, acute and chronic myelogenous leukemia and multiple myeloma. Other immune disorders include immunodeficiency, e.g., AIDS.

The use of GP286 modulators, including GP286 antisense reagents, GP286 ligands and anti-GP286 antibodies, to treat individuals having or at risk of developing such diseases is an aspect of the invention.

A composition of the invention typically contains from about 0.1 to 90% by weight (such as 1 to 20% or 1 to 10%) of a therapeutic agent of the invention in a pharmaceutically accepted carrier. Solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid: Disintegrators that can be used include, without limitation, microcrystalline cellulose, corn starch, sodium starch glycolate, and alginic acid. Tablet binders that can be used include acacia, methylcellulose, sodium carboxymethylcellulose, polyvinylpyrrolidone(Povidone™), hydroxypropyl methylcellulose, sucrose, starch and ethylcellulose. Lubricants that can be used include magnesium stearates, stearic acid, silicone fluid, talc, waxes, oils, and colloidal silica.

Liquid formulations of the compositions for oral administration prepared in water or other aqueous vehicles can contain various suspending agents such as methylcellulose, alginates, tragacanth, pectin, kelgin, carrageenan, acacia, polyvinylpyrrolidone, and polyvinyl alcohol. The liquid formulations can also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble versions of the compounds can be administered by the drip method, whereby a pharmaceutical formulation containing the antifungal agent and a physiologically acceptable excipient is infused. Physiologically acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

Inhalation and transdermal formulations can also readily be prepared.

Pharmaceutical formulation is a well-established art, and is further described in Gennaro (ed.), Remington: The Science and Practice of Pharmacy 20^{th} ed., Lippincott, Williams & Wilkins(2000) (ISBN: 0683306472); Ansel et al., Pharmaceutical Dosage Forms and Drug Delivery Systems, 7^{th} ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X), the disclosures of which are incorporated herein by reference in their entireties. Conventional methods, known to those of ordinary skill in the art of medicine, can be used to administer the pharmaceutical formulation(s) to the patient.

Typically, the pharmaceutical formulation will be administered to the patient by applying to the skin of the patient a transdermal patch containing the pharmaceutical formulation, and leaving the patch in contact with the patient's skin (generally for 1 to 5 hours per patch). Other transdermal routes of administration (e.g., through use of a topically applied cream, ointment, or the like) can be used by applying conventional techniques. The pharmaceutical formulation(s) can also be administered via other conventional routes (e.g., enteral, subcutaneous, intrapulmonary, transmucosal, intraperitoneal, intrauterine, sublingual intrathecal, or intramuscular routes) by using standard methods. In addition, the pharmaceutical formulations can be administered to the patient via injectable depot routes of administration such as by using 1-, 3-, or 6-month depot injectable or biodegradable materials and methods.

Regardless of the route of administration, the therapeutic protein or antibody agent typically is administered at a daily dosage of 0.01 mg to 30 mg/kg of body weight of the patient (e.g., 1 mg/kg to 5 mg/kg). The pharmaceutical formulation can be administered in multiple doses per day, if desired, to achieve the total desired daily dose. The effectiveness of the method of treatment can be assessed by monitoring the patient for known signs or symptoms of a disorder.

The pharmaceutical compositions of the invention may be included in a container, package or dispenser alone or as part of a kit with labels and instructions for administration.

Transgenic Animals and Cells

In another aspect, the invention provides transgenic cells and non-human organisms comprising gp286 nucleic acids, and transgenic cells and non-human organisms with targeted disruption of the endogenous ortholog of the human gp286 gene. The cells can be embryonic stem cells or somatic cells. The transgenic non-human organisms can be chimeric, non-chimeric heterozygotes, and non-chimeric homozygotes. The gp286 may be splice variant gp286 or gp286a.

Host cells of the invention may be used to produce non-human transgenic animals. For example, in some embodiments, a host cell of the invention is a fertilized oocyte or an embryonic stem cell into which gp286 nucleotide sequences have been introduced. Such a host cell may be used to create non-human transgenic animals in which exogenous gp286 sequences have been introduced into their genome or used to alter or replace related endogenous gp286 sequences in the animal.

As used herein, a “transgenic animal” is a non-human animal, preferably a mammal, more preferably a cow, goat, sheep or rodent such as a rat or mouse, in which one or more of the cells of the animal includes a transgene. Other examples of transgenic animals include non-human primates, dogs, chickens, amphibians, etc.

As used herein, a “transgene” is exogenous DNA that is integrated into the genome of a cell from which a transgenic animal develops and that remains in the genome of the mature animal, thereby directing the expression of an encoded gene product in one or more cell types or tissues of the transgenic animal.

As used herein, a “homologous recombinant animal” is a non-human animal, preferably a mammal, more preferably a mouse, in which an endogenous gp286 gene has been altered by homologous recombination between the endogenous gene and an exogenous DNA molecule introduced into a cell of the animal, e.g., an embryonic cell of the animal, prior to development of the animal

The non-human transgenic animals of the invention will be useful for studying the function and/or activity of gp286 and for identifying and/or evaluating modulators of gp286 activity. They will also be useful in methods for producing a GP286 protein or polypeptides fragment, i.e., in which the protein is produced in the mammary gland of a non-human mammal.

A transgenic animal of the invention can be created by introducing gp286-encoding nucleic acid into the male pronuclei of a fertilized oocyte, e.g., by microinjection, retroviral infection, and allowing the oocyte to develop in a pseudopregnant female foster animal. A polynucleotide comprising human gp286 DNA sequences of SEQ ID NO:1, SEQ ID NO:13, or SEQ ID NO: 14 may be introduced as a transgene into the genome of a non-human animal. Alternatively, a non-human homolog of the human gp286 gene, such as a mouse gp286 gene, isolated by hybridization to an isolated polynucleotide of the invention, may be used as a transgene. Heterologous transcription control sequence sequences, intronic sequences, polyadenylation signals and the like may also be operatively linked with the transgene to increase the efficiency or otherwise regulate the expression (e.g., in a developmental or tissue specific manner) the transgene in the recipient host animal.

Methods for generating transgenic animals via embryo manipulation and microinjection, particularly animals such as mice, have become conventional in the art and are described, for example, in U.S. Pat. Nos. 4,736,866; 4,870,009; and 4,873,191; Hogan 1986, in MANIPULATING THE MOUSE EMBRYO, Cold Spring Harbor Laboratory Press, Cold Spring Harbor, N.Y.; and Hogan 1999, in MANIPULATING THE MOUSE EMBRYO, 2d Ed., Cold Spring Harbor Laboratory Press, Cold Spring Harbor, N.Y. Similar methods are used for production of other transgenic animals. A transgenic founder animal can be identified based upon the presence of the gp286 transgene in its genome and/or expression of gp286 mRNA in tissues or cells of the animals. A transgenic founder animal can then be used to breed additional animals carrying the transgene. Moreover, transgenic animals carrying a transgene encoding gp286 can further be bred to other transgenic animals carrying other transgenes.

To create a homologous recombinant animal, a vector is prepared which contains at least a portion of a gp286 gene into which a deletion, addition or substitution has been introduced to thereby alter, e.g., functionally disrupt, the gp286 gene. The gp286 gene can be a human gene (e.g., SEQ ID NOs:1, 13 or 14), but more preferably, is a non-human homolog of a human gp286 gene. For example, a mouse homolog of the human gp286 gene of SEQ ID NOs:1, 13 or 14 can be used to construct a homologous recombination vector suitable for altering an endogenous gp286 gene in the mouse genome.

In some embodiments, the vector is designed such that, upon homologous recombination, the endogenous gp286 gene is functionally disrupted (i.e., no longer encodes a functional protein; also referred to as a “knock out” vector). Alternatively, the vector can be designed such that, upon homologous recombination, the endogenous gp286 gene is mutated or otherwise altered but still encodes functional protein (e.g., the upstream regulatory region can be altered to thereby alter the expression of the endogenous GP286 protein). In the homologous recombination vector, the altered portion of the gp286 gene is flanked at its 5′ and 3′ ends by additional nucleic acid of the gp286 gene to allow for homologous recombination to occur between the exogenous gp286 gene carried by the vector and an endogenous gp286 gene in an embryonic stem cell. The additional flanking gp286 nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for an exemplary description of homologous recombination vectors.

The vector is introduced into an embryonic stem cell line (e.g., by electroporation) and cells in which the introduced gp286 gene has homologously recombined with the endogenous gp286 gene are selected (see e.g., Li et al. (1992) Cell 69:915). The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene.

Methods for constructing homologous recombination vectors and homologous recombinant animals are described further in Bradley (1991) Curr. Opin. Biotechnol. 2:823-829; Hogan 1999, in MANIPULATING THE MOUSE EMBRYO, 2d Ed., Cold Spring Harbor Laboratory Press, Cold Spring Harbor, N.Y.; PCT International Publication Nos.: WO 90/11354; WO 91/01140; WO 92/0968; and WO 93/04169;

Clones of the non-human transgenic animals described herein can also be produced according to the methods described, e.g., in Wilmut et al. (1997) Nature 385:810-813. In brief, a cell, e.g., a somatic cell, from the transgenic animal can be isolated and induced to exit the growth cycle and enter GO phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Regulated expression of transgenes in vivo may be accomplished using controllable recombination systems, such as the cre/loxP recombinase system (see, e.g., Lakso et al. (1992) Proc. Natl. Acad. Sci. USA 89:6232-6236) and the FLP recombinase system(O'Gorman et al. (1991) Science 251:1351-1355. If a cre/loxP recombinase system is used to regulate expression of the transgene, animals containing transgenes encoding both the Cre recombinase and a selected protein are required. Transgenic animals containing both elements of the system can be obtained, e.g., by mating two transgenic animals, each containing either the transgene encoding the selected protein or the transgene encoding a recombinase.

Antisense Reagents and Methods

A Antisense

Many of the isolated polynucleotides of the invention are antisense polynucleotides that recognize and hybridize to gp286 polynucleotides. Full-length and fragment antisense polynucleotides are provided. Fragment antisense molecules of the invention include (i) those that specifically recognize and hybridize to gp286 RNA (as determined by sequence comparison of DNA encoding GP286 to DNA encoding other known molecules). Identification of sequences unique to GP286 encoding polynucleotides can be deduced through use of any publicly available sequence database, and/or through use of commercially available sequence comparison programs. After identification of the desired sequences, isolation through restriction digestion or amplification using any of the various polymerase chain reaction techniques well known in the art can be performed. Antisense polynucleotides are particularly relevant to regulating expression of GP286 by those cells expressing gp286 mRNA, e.g., Tcells.

Antisense oligonucleotides, or fragments of a nucleotide sequence set forth in SEQ ID NOs: 1, 13 or 14, or sequences complementary or homologous thereto, derived from the nucleotide sequences encoding GP286 are useful as diagnostic tools for probing gene expression in various tissues. For example, tissue can be probed in situ with oligonucleotide probes carrying detectable groups by conventional autoradiography techniques to investigate native expression of this enzyme or pathological conditions relating thereto. In specific aspects, antisense nucleic acid molecules are provided that comprise a sequence complementary to at least about 10, 25, 50, 100, 250 or 500 nucleotides or an entire gp286 coding strand, or to only a portion thereof. Nucleic acid molecules encoding fragments, homologs, derivatives and analogs of a GP286 protein of SEQ ID NOs:2 or 15, antisense nucleic acids complementary to a GP286 nucleic acid sequence of SEQ ID NOs:1, 13 or 14 are additionally provided.

Antisense nucleic acid molecules of the invention may be antisense to a “coding region” or non-coding regions of the coding strand of a nucleotide sequence encoding GP286. The term “coding region” refers to the region of the nucleotide sequence comprising codons which are translated into amino acid residues (e.g., a protein coding region of human GP286 corresponds to the coding region SEQ ID NOs:1 or 14).

Antisense oligonucleotides are preferably directed to a regulatory region of a nucleotide sequence of SEQ ID NOs:1, 13 or 14, or mRNA corresponding thereto, including, but not limited to, the initiation codon, TATA box, enhancer sequences, and the like. The antisense nucleic acid molecule can be complementary to the entire coding or non-coding region of gp286, but more preferably is an oligonucleotide that is antisense to only a portion of the coding or non-coding region of gp286 mRNA For example, the antisense oligonucleotide can be complementary to the region surrounding the translation start site of gp286 mRNA An antisense oligonucleotide can be, for example, about 5, 10, 15, 20, 25, 30, 35, 40, 45 or 50 nucleotides in length.

Antisense nucleic acids of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention (preferably oligonucleotides of 10 to 20 nucleotides in length) are typically administered to a subject or generated in situ such that they hybridize with or bind to cellular mRNA and/or genomic DNA encoding a GP286 protein to thereby inhibit expression of the protein, e.g., by inhibiting transcription and/or translation. Suppression of gp286 expression at either the transcriptional or translational level is useful to generate cellular or animal models for diseases/conditions characterized by aberrant gp286 expression. Suppression of gp286 expression is also useful to inhibit aberrant T cell adhesion to other cells or to extracellular matrices.

The hybridization can be by conventional nucleotide complementarity to form a stable duplex, or, for example, in the case of an antisense nucleic acid molecule that binds to DNA duplexes, through specific interactions in the major groove of the double helix. Phosphorothioate and methylphosphonate antisense oligonucleotides are specifically contemplated for therapeutic use by the invention. The antisense oligonucleotides may be further modified by adding poly-L-lysine, transferrin, polylysine, or cholesterol moieties at their 5′ end.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In other embodiments, the antisense nucleic acid molecule of the invention is an a-anomeric nucleic acid molecule. An a-anomeric nucleic acid molecule forms specific double-stranded hybrids with complementary RNA in which, contrary to the usual b-units, the strands run parallel to each other (Gaultier et al. (1987) Nucleic Acids Res 15: 6625-6641). The antisense nucleic acid molecule can also comprise a 2′-O-methylribonucleotide (Inoue et al. (1987) Nucleic Acids Res 15: 6131-6148) or a chimeric RNA-DNA analogue (Inoue et al. (1987) FEBS Lett 215: 327-330).

B. Ribozymes and Catalytic Nucleic Acids

In some embodiments, an antisense nucleic acid of the invention is part of a gp286 specific ribozyme (or, as modified, a “nucleozyme”). Ribozymes are catalytic RNA molecules with ribonuclease activity that are capable of cleaving a single-stranded nucleic acid, such as an mRNA, to which they have a complementary region. Thus, ribozymes (such as hammerhead, hairpin, Group I intron ribozymes, and the like) can be used to catalytically cleave gp286 mRNA transcripts to thereby inhibit translation of gp286 mRNA. A ribozyme having specificity for a gp286-encoding nucleic acid can be designed based upon the nucleotide sequence of a gp286 polynucleotide disclosed herein (SEQ ID NOs: 1, 13 or 14). See, e.g., U.S. Pat. Nos. 5,116,742; 5,334,711; 5,652,094; and 6,204,027, incorporated herein by reference in their entireties.

For example, a derivative of a Tetrahymena L-19 IVS RNA can be constructed in which the nucleotide sequence of the active site is complementary to the nucleotide sequence to be cleaved in a GP286-encoding mRNA See, e.g., Cech et al. U.S. Pat. No. 4,987,071; and Cech et al. U.S. Pat. No. 5,116,742. Alternatively, gp286 mRNA can be used to select a catalytic RNA having a specific ribonuclease activity from a pool of RNA molecules. See, e.g., Bartel et al., (1993) Science 261:1411-1418.

Expression of the gp286 gene may be inhibited by targeting nucleotide sequences complementary to the regulatory region of the gp286 (e.g., the gp286 promoter and/or enhancers) to form triple helical structures that prevent transcription of the gp286 gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

C. Peptide Nucleic Acids (PNA)

In other preferred oligonucleotide mimetics, especially useful for in vivo administration, both the sugar and the internucleoside linkage are replaced with novel groups, such as peptide nucleic acids (PNA). See, e.g., Hyrup et al. (1996) Bioorg. Med. Chem. Lett. 4:5-23. In PNA compounds, the phosphodiester backbone of the nucleic acid is replaced with an amide-containing backbone, in particular by repeating N-(2-aminoethyl) glycine units linked by amide bonds. Nucleobases are bound directly or indirectly to aza-nitrogen atoms of the amide portion of the backbone, typically by methylene carbonyl linkages. The synthesis of PNA oligomers can be performed using standard solid phase peptide synthesis protocols as described in Hyrup et al., supra; and Perry-O'Keefe et al., Proc. Natl. Acad. Sci. USA 93:14670-675 (1996).

PNAs of gp286 can be used in therapeutic and diagnostic applications. For example, PNAs can be used as antisense or antigene agents for sequence-specific modulation of gene expression by, e.g., inducing transcription or translation arrest or inhibiting replication. PNAs of gp286 can also be used, e.g., in the analysis of single base pair mutations in a gene by, e.g., PNA directed PCR clamping; as artificial restriction enzymes when used in combination with other enzymes, e.g., S1 nucleases; or as probes or primers for DNA sequence and hybridization (Hyrup et al., supra; and Perry-O'Keefe, supra).

In some embodiments, PNAs of gp286 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of gp286 can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, supra). The synthesis of PNA-DNA chimeras can be performed as described in Hyrup., supra and Finn et al., Nuc. Acids Res. 24:3357-63 (1996).

For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al., Nuc. Acids Res. 17:5973-88 (1989)). PNA monomers are then coupled in a stepwise manner to produce a chimeric molecule with a 5′ PNA segment and a 3′ DNA segment (Finn et al., supra). Alternatively, chimeric molecules can be synthesized with a 5′ DNA segment and a 3′ PNA segment. See, Petersen et al., Bioorg. Med. Chem. Lett. 5:1119-11124 (1975).

In other embodiments, the oligonucleotide may include other appended groups such as peptides (e.g., for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT Publication No. WO88/09810) or the blood-brain barrier (see, e.g., PCT Publication No. WO89/10134). In addition, oligonucleotides can be modified with hybridization triggered cleavage agents (See, e.g., Krol et al., BioTechniques 6:958-976 (1988)), or intercalating agents (See, e.g., Zon, Pharm. Res. 5: 539-549 (1988)). To this end, the oligonucleotide may be conjugated to another molecule, e.g., a peptide, a hybridization triggered cross-linking agent, a transport agent, a hybridization-triggered cleavage agent, etc.

PNA chemistry and applications are reviewed, inter alia, in Ray et al., FASEB J 14(9): 1041-60 (2000); Nielsen et al., Pharmacol Toxicol. 86(1):3-7 (2000); Larsen et al., Biochim Biophys Acta 1489(1):159-66 (1999); Nielsen, Curr. Opin. Struct. Biol. 9(3):353-7 (1999), and Nielsen, Curr. Opin. Biotechnol. 10(1):71-5 (1999), the disclosures of which are incorporated herein by reference in their entireties.

Diagnostic Methods

A. Nucleic Acid Diagnostics

As described above, the isolated polynucleotides of the present invention can be used as nucleic acid probes to assess the levels of gp286 mRNA in tissues in which it is normally expressed (e.g., immune system tissues, particularly in T cells), and in tissues in which it is not normally expressed at significant levels, if such abnormal tissue mis-expression is suspected.

The present invention thus provides a method for detecting the presence of a gp286 polynucleotide in a biological sample (e.g., a cell extract, fluid or tissue sample derived from a patient) by contacting the sample with an isolated polynucleotide of the invention which is capable of specifically detecting by hybridization gp286 polynucleotide sequences. This method is useful, e.g., for determining whether a non-immune tissue has been infiltrated with T cells, which may be indicative of inflammation and/or disease.

Preferably, the method comprises the steps of contacting the sample with an the isolated nucleic acid under high stringency hybridization conditions and detecting hybridization of the isolated polynucleotide to a nucleic acid in the sample, wherein the occurrence of said hybridization indicates the presence of a gp286-encoding sequence in the sample.

The present invention also provides a diagnostic assay for identifying the presence or absence of a genetic lesion or mutation characterized by at least one of: (i) aberrant modification or mutation of a gene encoding a GP286 protein; (ii) mis-regulation of a gene encoding a GP286 protein; and (iii) aberrant post-translational modification of a GP286 protein, wherein a wild-type form of the gene encodes a protein with a GP286 biological activity.

The present invention further provides a method of identifying a homolog of a human gp286 gene, comprising the step of hybridizing a nucleic acid library with a nucleic acid probe comprising SEQ ID NOs:1, 13 or 14, or a portion thereof having at least 17 nucleotides, under high stringency hybridization conditions; and determining whether the nucleic acid probe hybridizes to a nucleic acid sequence in the library. If the nucleic acid sequence in the library hybridizes under such selected conditions, it is a homolog of a human gp286 gene.

B. Antibody Diagnostics

Antibodies of the present invention can be used to assess the expression levels of GP286 proteins in tissues in which it is normally expressed (e.g., immune system tissues), and in tissues in which it is not normally expressed, if such abnormal tissue mis-expression is suspected.

The present invention thus provides a method for detecting the presence of a GP286 protein or its activity in a biological sample (e.g., a cell extract, fluid or tissue sample derived from a patient) by contacting the sample with an agent capable of detecting an indicator of the presence of GP286 protein or its activity. Preferably, the agent is an antibody specific for at least one epitope of GP286 protein. In more preferred embodiments, the antibody is specific for one or the other splice variant—GP286 or GP286a.

Accordingly, the invention provides a method for determining whether a GP286 protein is present in a sample, comprising the step of contacting the sample with an antibody having at least one GP286 epitope and detecting specific binding of the antibody to an antigen, which indicates the presence of a GP286 protein in the sample. This method is useful for, e.g., determining whether a non-immune tissue has been infiltrated by T cells, which may be indicative of inflammation or other disease state.

C. Methods for Diagnosing Disease

The gp286 isolated polynucleotides, proteins and GP286 specific antibodies of the invention will be useful in methods for diagnosing a variety of disorders and disease conditions associated with aberrant gp286 expression.

The invention thus provides a method for diagnosing a disease condition in a subject, comprising the steps of comparing the amount or activity of a GP286 protein in a tissue sample from the subject to the amount or activity of the GP286 polypeptide in a control sample (e.g., an equivalent one derived from a healthy subject), wherein a significant difference in the amount or activity of the GP286 polypeptide in the tissue sample relative to the amount or activity of the GP286 polypeptide in the control sample indicates that the subject has a disease condition.

In preferred embodiments, the amount or activity of a GP286 protein in a tissue sample is assessed by competitive binding assays using a GP286 polypeptides or fragment of the invention, or by an immunoassay using a GP286 specific antibody of the invention. Preferably, the method is used to diagnose a disease condition relating to the pancreas or to the nervous system.

Also provided are methods for diagnosing a disease condition in a subject by monitoring relative gp286 mRNA levels in difference tissues. Preferably, the methods comprise the step of comparing the amount of a gp286 mRNA in a test tissue sample from the subject to the amount of gp286 mRNA in a control sample, wherein a significant difference in the amount of the mRNA in the test sample relative to the amount in the control sample indicates that the subject has a disease condition.

In preferred embodiments, the amount of gp286 mRNA in a tissue sample is assessed by hybridization using an isolated gp286 polynucleotide or nucleic acid fragment of the invention. Preferably, the method is used to diagnose a disease condition relating to the immune system, particularly to one involving T cells.

Computer Readable Means

A further aspect of the invention is a computer readable means for storing the gp286 nucleic acid and amino acid sequences of the instant invention. In preferred embodiments, the invention provides a computer readable means for storing SEQ ID NOS:1-33 as described herein, as the complete set of sequences or in any combination The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

The nucleic acid and amino acid sequences of the invention are particularly useful as components in databases useful for search analyses as well as in sequence analysis algorithms. As used in these embodiments, the terms “nucleic acid sequences of the invention” and “amino acid sequences of the invention” mean any detectable chemical or physical characteristic of a polynucleotide or polypeptide of the invention that is or may be reduced to or stored in a computer readable form. These include, without limitation, chromatographic scan data or peak data, photographic data or scan data therefrom, and mass spectrographic data.

This invention provides computer readable media having stored thereon sequences of the invention. A computer readable medium may comprise one or more of the following: a nucleic acid sequence comprising a sequence of a nucleic acid sequence of the invention; an amino acid sequence comprising an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of one or more nucleic acid sequences of the invention; a data set representing a nucleic acid sequence encoding an amino acid sequence comprising the sequence of an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of a nucleic acid sequence of the invention; a data set representing a nucleic acid sequence encoding an amino acid sequence comprising the sequence of an amino acid sequence of the invention. The computer readable medium can be any composition of matter used to store information or data, including, for example, commercially available floppy disks, tapes, hard drives, compact disks, and video disks.

Accordingly, the invention provides a diagnostic assay for identifying a homolog of a human gp286 gene, comprising the step of screening a nucleic acid database with a query sequence consisting of SEQ ID NOs:1, 13 or 14, or a portion thereof having 300 or more nucleotides, wherein a nucleic acid sequence in said database that is at least 65% but less than 100% identical to SEQ ID NOs:1, 13 or 14, or said portion thereof if found, is a homolog of a human gp286 gene.

Also provided by the invention are methods for the analysis of character sequences, particularly genetic sequences of the invention. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, and sequencing chromatogram peak analysis.

A computer-based method is provided for performing nucleic acid homology identification. This method comprises the steps of providing a nucleic acid sequence comprising the sequence of a nucleic acid of the invention in a computer readable medium; and comparing said nucleic acid sequence to at least one nucleic acid or amino acid sequence to identify homology.

A computer-based method is also provided for performing amino acid homology identification, said method comprising the steps of: providing an amino acid sequence comprising the sequence of a polypeptide of the invention in a computer readable medium; and comparing said amino acid sequence to at least one nucleic acid or an amino acid sequence to identify homology.

A computer based method is still further provided for assembly of overlapping nucleic acid sequences into a single nucleic acid sequence, said method comprising the steps of providing a first nucleic acid sequence comprising the sequence of a nucleic acid of the invention in a computer readable medium; and screening for at least one overlapping region between said first nucleic acid sequence and a second nucleic acid sequence.

### EXAMPLES

The following example is meant to illustrate the methods and materials of the present invention. Suitable modifications and adaptations of the described conditions and parameters normally encountered in the art of molecular biology which are apparent to those skilled in the art are within the spirit and scope of the present invention.

For the experiments described below, all RT-PCR and fragments were gel-purified prior to cloning. The fragments were separated by agarose gel electrophoresis by standard methods. DNA fragments were excised from the agarose gel and purified from the gel using QIAEX resin according to the manufacturer's specifications (Qiagen, Valencia, Calif.). The gel-purified fragments were cloned into plasmid vectors and then the plasmids were used to transform competent TOP10 E. coli host cells. Plasmids produced by the host cells were isolated by a standard alkaline lysis miniprep procedure (Qiagen, Valencia, Calif.). Sequencing was executed by a standard dideoxy termination method (Applied Biosystems, Foster City, Calif.).

### Example 1

### Gene Prediction and Sequence Analysis

Gene prediction programs were used to identify novel genes in high throughput genomic sequence data deposited in GenBank. High throughput genomic sequence data from GenBank were analyzed using the gene prediction programs GENSCAN (Burge and Karlin, 1997, J. Mol. Biol. 268:78-94, incorporated herein by reference in its entirety) and GENEMARKHMM (Lukashin and Borodovsky, 1998, Nuc. Acids Res. 26:1107-1115, incorporated herein by reference in its entirety), the use of which is familiar to those skilled in the art. The GenBank data entries were downloaded to a local server, individual sequence contigs were separated according to the annotation provided with the sequence entries, and GENSCAN and GENEMARK were used to predict genes. The GENSCAN and GENEMARKHMM parameters used were the default parameters included with the programs (Burge and Karlin, 1997; Lukashin and Borodovsky, 1998). Those genes for which both gene prediction programs yielded similar results were further analyzed. Specifically, the gene sequences were translated to protein and the protein sequences were used as queries in BLAST analyses of the Genpept and Swissprot protein sequence databases.

The BLAST algorithm, which stands for Basic Local Alignment Search Tool is suitable for determining sequence similarity (Altschul et al., J. Mol. Biol. 1990, 215, 403410, which is incorporated herein by reference in its entirety). Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (13) of 50, expectation (E) of 10, M=5, N=4, and a comparison of both strands.

BLAST (Karlin et al., Proc. Natl. Acad. Sci. USA, 1993, 90, 5873-5787, which is incorporated herein by reference) and Gapped BLAST perform a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a nucleic acid is considered similar to a GP286 gene or cDNA if the smallest sum probability in comparison of the test nucleic acid to a GP286 nucleic acid is less than about 1, preferably less than about 0.1, more preferably less than about 0.01, and most preferably less than about 0.001.

GP286 was identified in contig 49 of a BAC with the GenBank accession number AC008616, which was deposited on Aug. 3, 1999. The genscan prediction for this gene was in the reverse orientation and included the following exons: exon 1 (initial exon), 53332-53287; exon 2, 49292-48933; exon 3, 45763-45722; exon 4, 45627-45514; exon 5 (terminal exon), 45368-45334. The GENEMARK prediction was also in the reverse orientation and included the following exons: exon 1 (initial exon), 53317-53287; exon 2, 49292-48933; exon 3, 45763-45722; exon 4 (terminal exon), 45692-45652. It was noted that exons 1, 2, and 3 overlapped either partially or completely for the two gene predictions. Blast analysis of this clone against publicly available EST databases showed no ESTs that matched the predicted gene.

### Example 2

### Amplification of gp286

The fill-length sequence was obtained by performing Rapid Amplification of cDNA Ends (RACE) using the Marathon-Ready RACE kit (Clontech, Palo Alto). Marathon-Ready™ cDNA is a double stranded cDNA synthesized from human tissue mRNA and ligated to a standard set of adapters (Clontech, Palo Alto). All RACE reactions used the adapter primer AP-1, 5′-CCATCCTAATACGACTCACTATAGGGC-3′ (SEQ ID NO:11) provided with the kit. The 5′ RACE for GP286 used AP-1 together with CR2-146, 5′-TGTTGCCCTCAGCCTCCTCCAACTCA-3′ (SEQ ID NO:9). The 3′ RACE for GP286 used AP-1 (SEQ ID NO:11) together with CR2-148, 5′-ACCCCACACAGAACAGAAACCGGATCG-3′ (SEQ ID NO: 10). Advantaq 2 DNA polymerase (Clontech, Palo Alto) was used for the amplification reactions. The Marathon-Ready™ kit was used according to the manufacturer's specifications except that “touchdown” PCR (Don et al., 1991. Nuc. Acids Res. 19:4008 (incorporated herein by reference in its entirety)) conditions were used for thermal cycling. The thermal cycling conditions were as follows: 94° C. for 1 minute, one cycle of 94° C. for 15 seconds, 72° C. for 15 seconds, 68° C. for 15 seconds; one cycle of 94° C. for 15 seconds, 71° C. for 15 seconds, 68° C. for 15 seconds; one cycle of 94° C. for 15 seconds, 70° C. for 15 seconds, 68° C. for 15 seconds; one cycle of 94° C. for 15 seconds, 69° C. for 15 seconds, 68° C. for 15 seconds; 35 cycles of 94° C. for 15 seconds and 68° C. for 30 seconds; and 68° C. for 10 minutes.

### Example 3

### Confirmation of GP286 Expression by RT-PCR

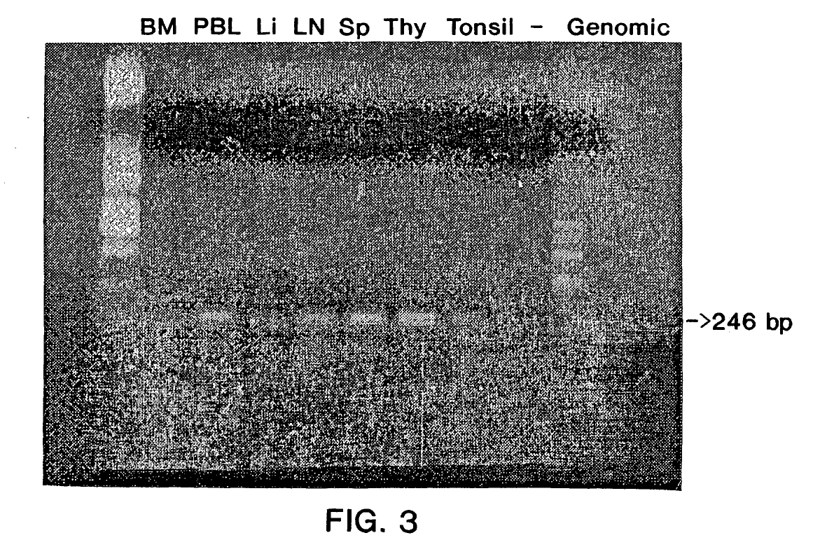

RT-PCR was used to confirm that the predicted gene was expressed and to initiate the cloning process that would determine the true (rather than the predicted) gene structure. The primers CR2-037 (GENESCAN exon 2, coordinates 49110-49130) 5′-ACATCACCAACGGCAGCCTCA-3′ (SEQ ID NO:7), and CR2-038 (GENESCAN exon 3, coordinates 45730-45750) 5′-CTTGCGATCCGGTTTCTGTTC-3′ (SEQ ID NO:8) were used as amplification primers on template multipe tissue cDNA panels (Clontech, Palo Alto) according to the manufacturer's specifications. The multiple tissue panels provided double-stranded human cDNA as templates for PCR, and included the following tissues: bone marrow, peripheral blood leukocytes, liver, lymph node, spleen, thymus, and tonsil. Thermal cycler conditions for the RT-PCR were: 94° C. for 1 minute, followed by 35 cycles of 94° C. for 15 seconds, 60° C. for 15 seconds, 72° C. for 15 seconds, followed by 2 minutes at 72° C. Bands of roughly 220-250 bp in including peripheral blood leukocytes, lymph nodes, spleen, thymus and tonsil indicate that GP286 is expressed in these immune system tissues (FIG. 3). That these bands are derived from spliced cDNA rather than from genomic DNA contamination of the RT-PCR is indicated by the size of the bands: a genomic band amplified with these two primers would be approximately 3400 bp in length rather than the observed 220-250 bases. The PCR fragment from the thymus was excised from the gel, cloned, and sequenced (SEQ ID NO:3).

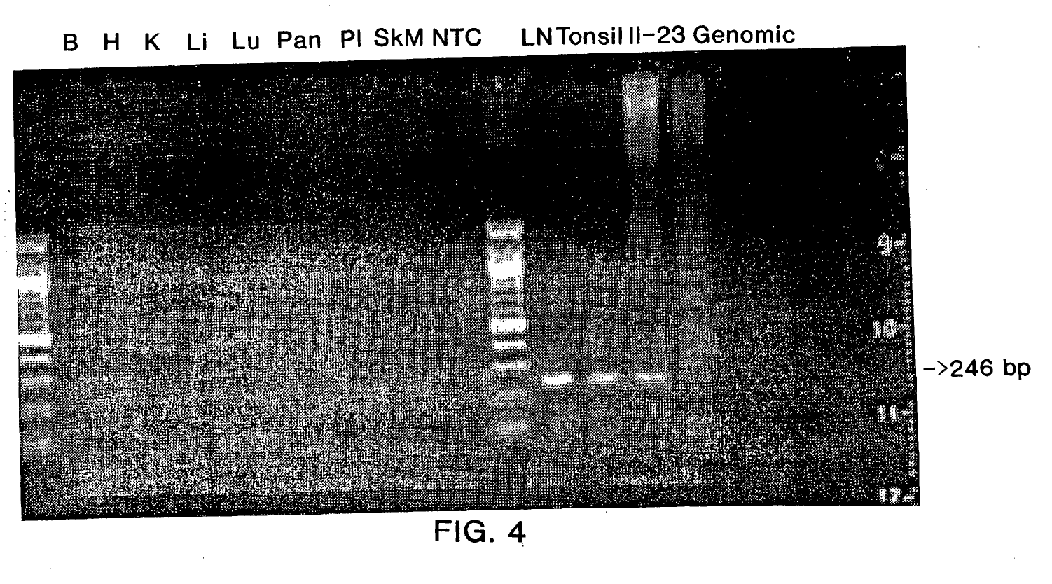

In another experiment immune and non-immune tissues were subject to RT-PCR as described above. In this experiment, the cDNA used were derived from the following tissues: brain, heart, kidney, liver, lung, pancreas, placenta, skeletal muscle, lymph node, tonsil, and mouse T cell line II-23. A no template control reaction and a genomic DNA template control reaction were also performed. As shown in FIG. 4, although gp286 is detectable in a number of non-immune tissues, gp286 was much more highly expressed in immune tissues than non-immune tissues. It is believed that gp286 is observed in non-immune tissues due to the presence of infiltrating T cells in those tissues or organs.

### Example 4

### Identification of Full-Length gp286 cDNA by RACE

Since the gene prediction programs GENSCAN and GENEMARK have predictable error rates (Burge and Karlin, 1997; Lukashin and Borodovsky, 11998), the RT-PCR fragment generated by amplification with CR2-037 and CR2-038 was used as a seed sequence to obtain the rest of the cDNA sequence for. GP286. Rapid Amplification of cDNA Ends (RACE) was used to determine the remainder of the cDNA sequence encoded by GP286. For the 5′ RACE reaction, the primer was CR2-146 (5′-TGTTGCCCTCAGCCTCCTCCAACTCA-3′) (SEQ ID NO:9) and the template was cDNA synthesized from human thymus tissue (see Example 3). For the 3′ RACE the primer was CR2-148 (5′-ACCCCACACAGAACAGAAACCGGATCG-3) (SEQ ID NO:10) and the template was cDNA synthesized from human spleen tissue. The 5′ RACE yielded a fragment of 450 base pairs. This fragment was gel purified, cloned, and sequenced (SEQ ID NO:4). The sequence of the fragment overlapped with the original PCR product and extended the cDNA sequence at the 5′ end to the start codon and into the 5′ un-translated region (UTR). The 3′ RACE product was 550 bp in length and extended through the stop codon and into the 3′ UTR This fragment was gel purified, cloned, and sequenced (SEQ ID NO:5).

To assemble the full-length GP286 sequence the initial PCR product, the 5′ RACE product and the 3′ RACE product were assembled into a single contiguous sequence using the ASSEMBLE program in the GCG™ computer package (Genetics Computer Group, Madison, Wis.). The assembled sequence is shown in Table 4 and SEQ ID NO:1.

### Example 5

### Confirmation of GP286 Expression by Northern Blot Analysis

A Northern blot was purchased from Clontech (Cat. No. 7790-1) which contained RNA from human cell lines of various origins, including: HL-60 (promyelocytic leukemia); HeLa (cervical adenocarcinoma); K-562 (chronic myelogenous leukemia); MOLT-4 (lymphoblastic leukemia); Raji (Burkitt's lymphoma); SW480 (colorectal adenocarcinoma) A549 (lung carcinoma); and G-361 (melanoma).

The blot was probed with a 232 bp fragment of gp286 (SEQ ID NO:3). The probe was prepared from 50 ng of cDNA labeled by a random-primed method (Feinberg and Vogelstein, 1983, A method of labeling DNA restriction fragments to high specific activity. Anal. Biochem. 132:6-13). Hybridization was carried out at 68° C. for 1 hour in ExpressHyb™ solution (Clontech, Cat. No. 8015-1) followed by washing with 2×SSC/0.05% SDS at room temperature and two washes with 0.1×SSC/0.1% SDS at 50° C.

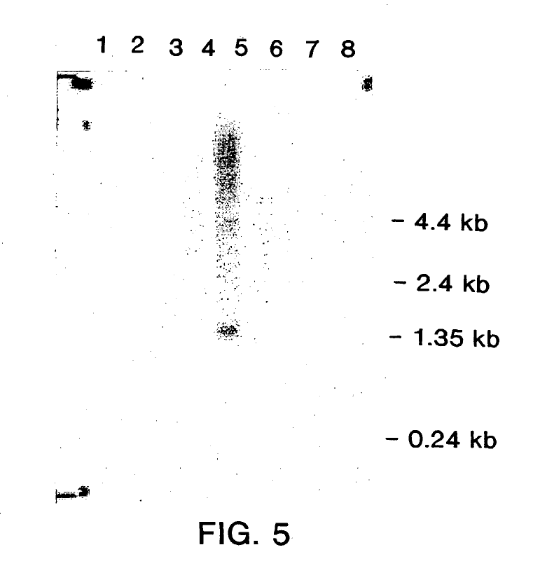

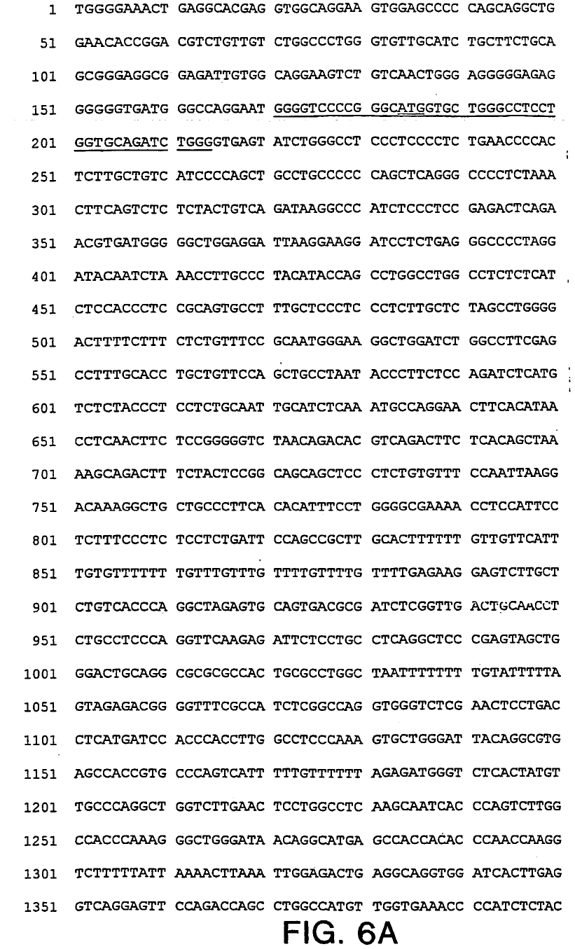

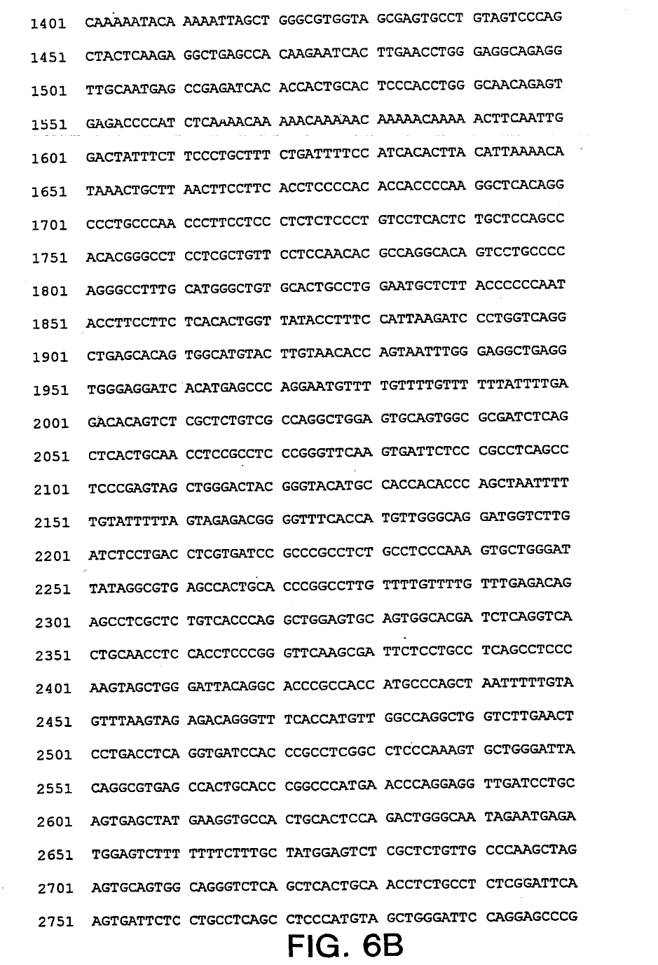

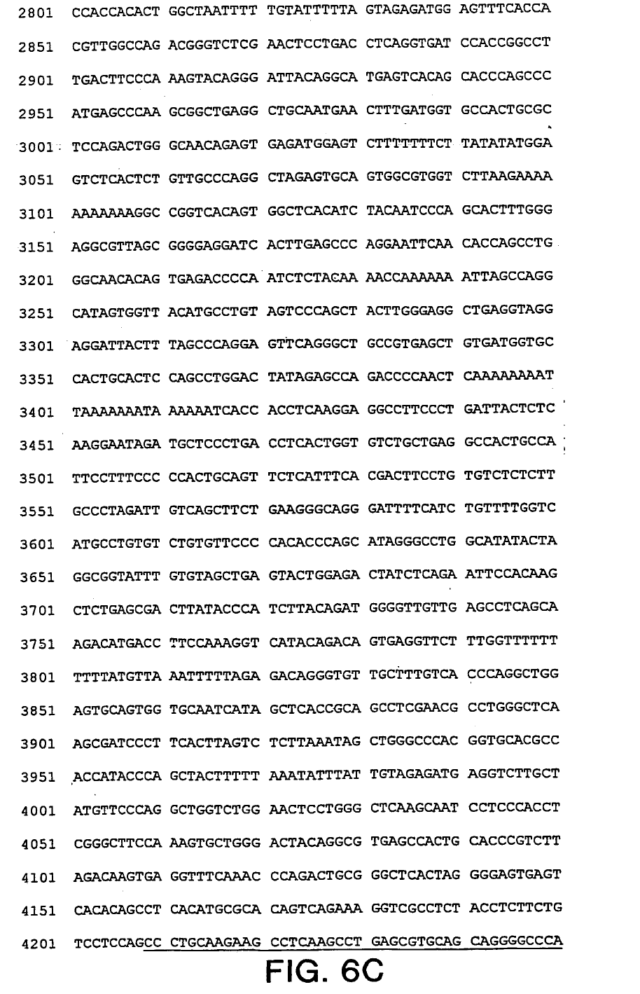

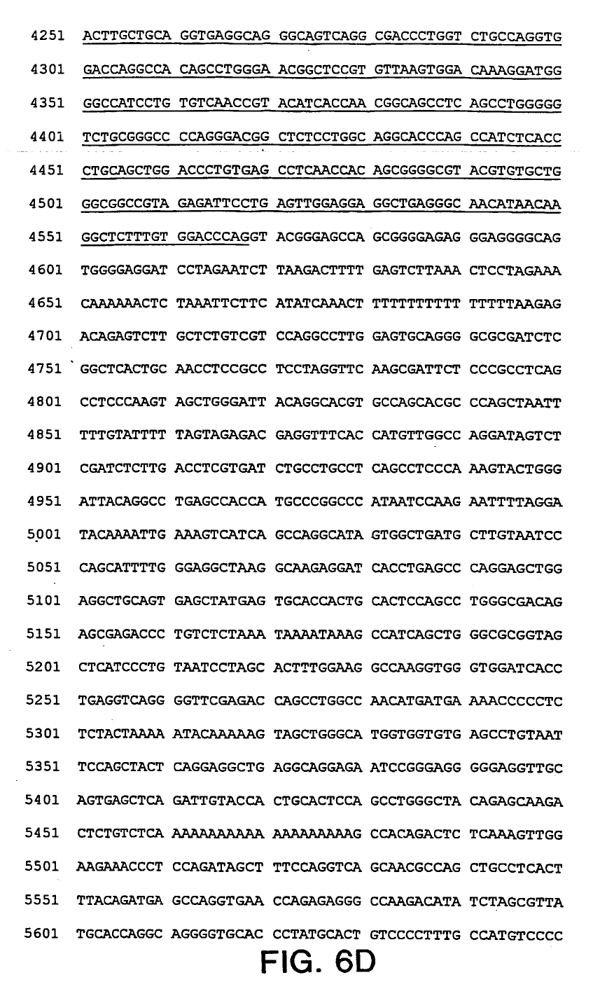

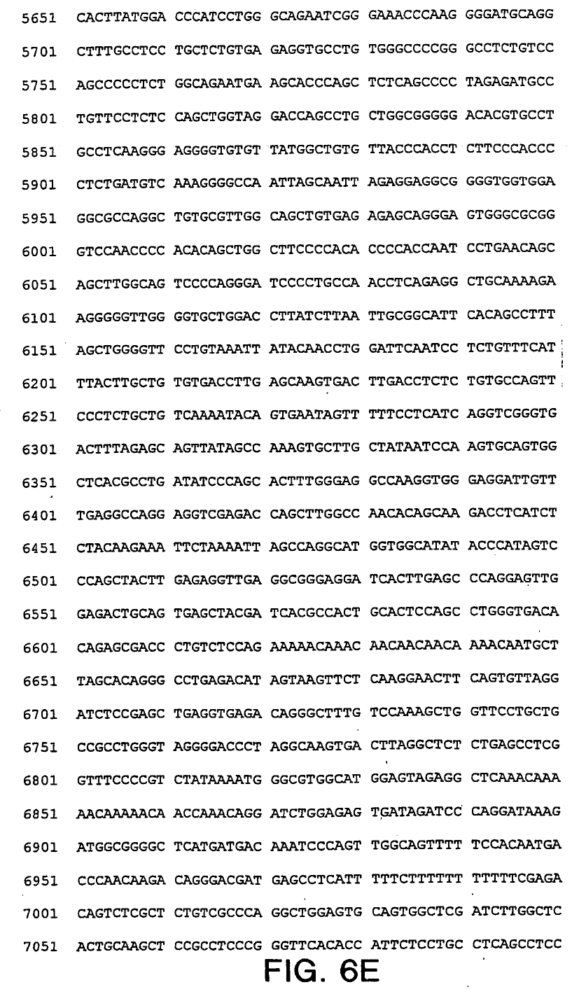

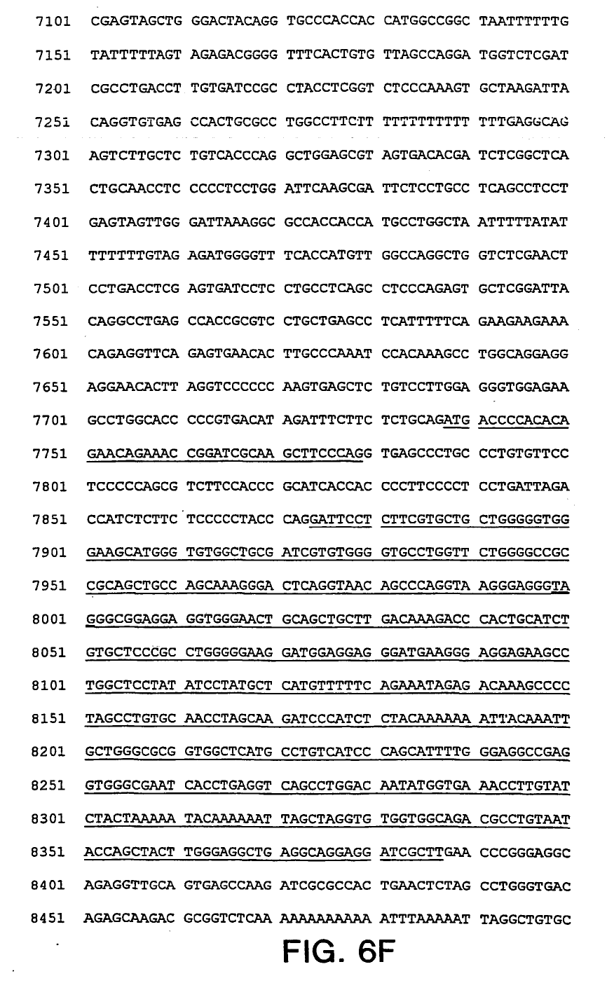

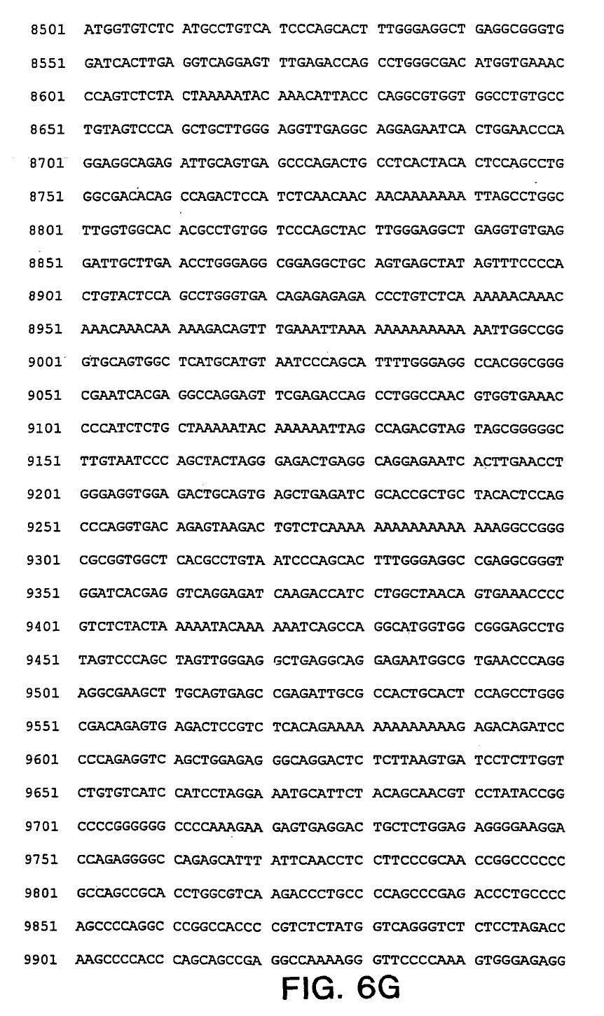

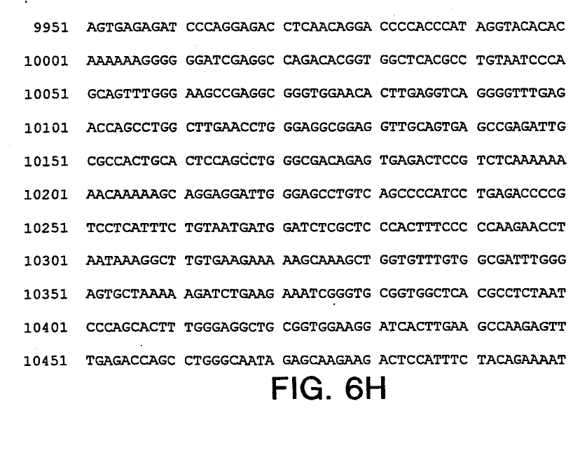

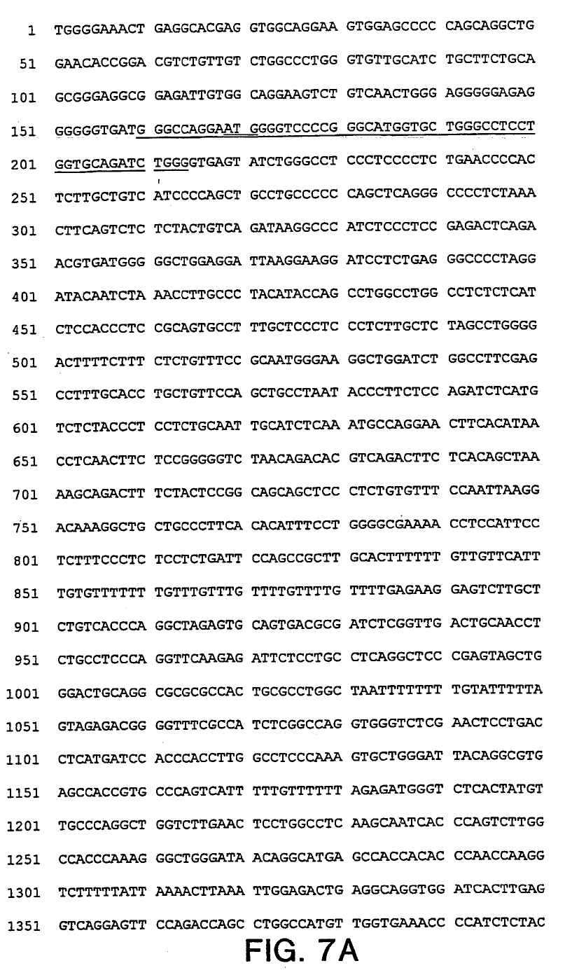

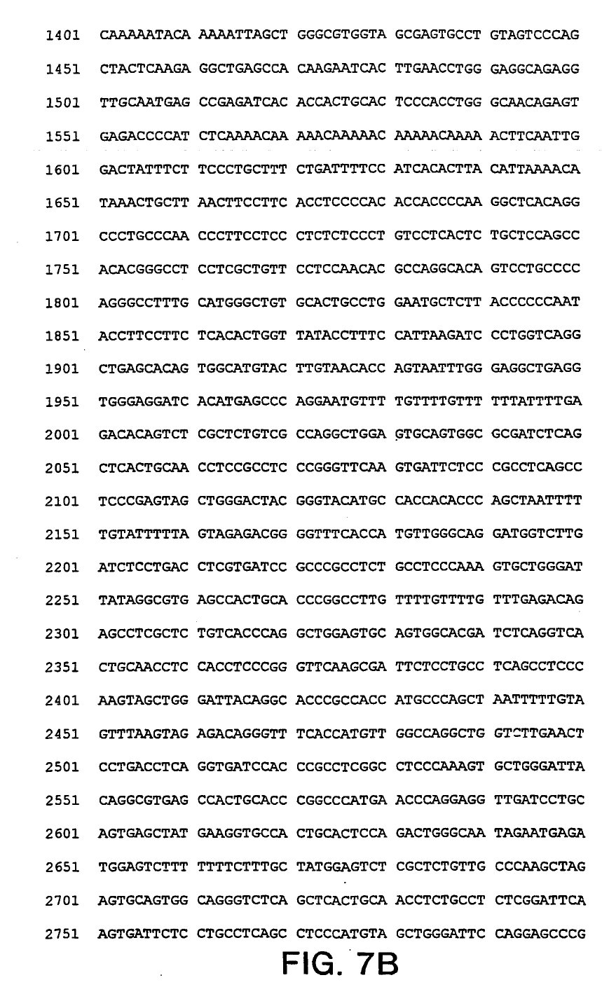

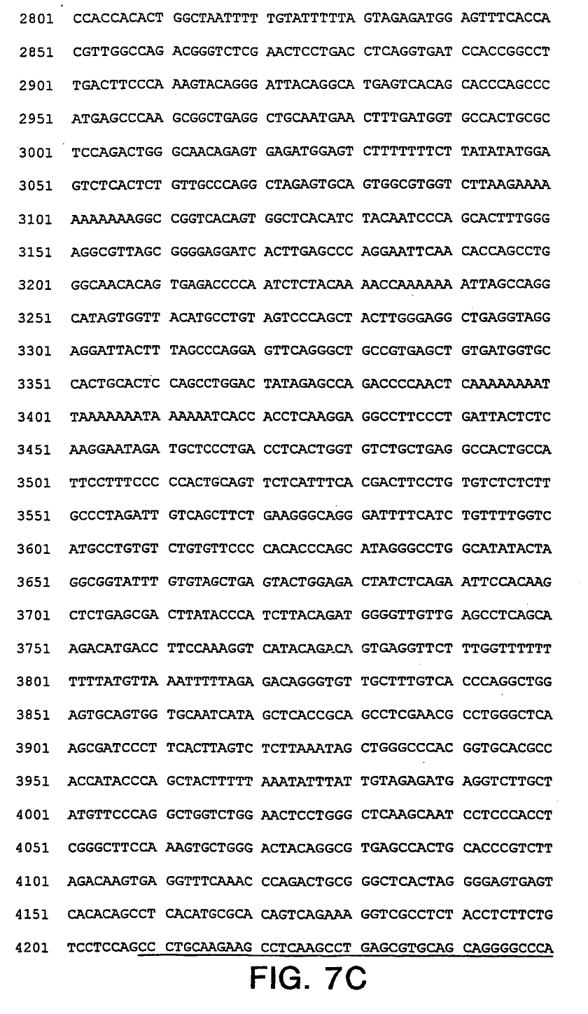

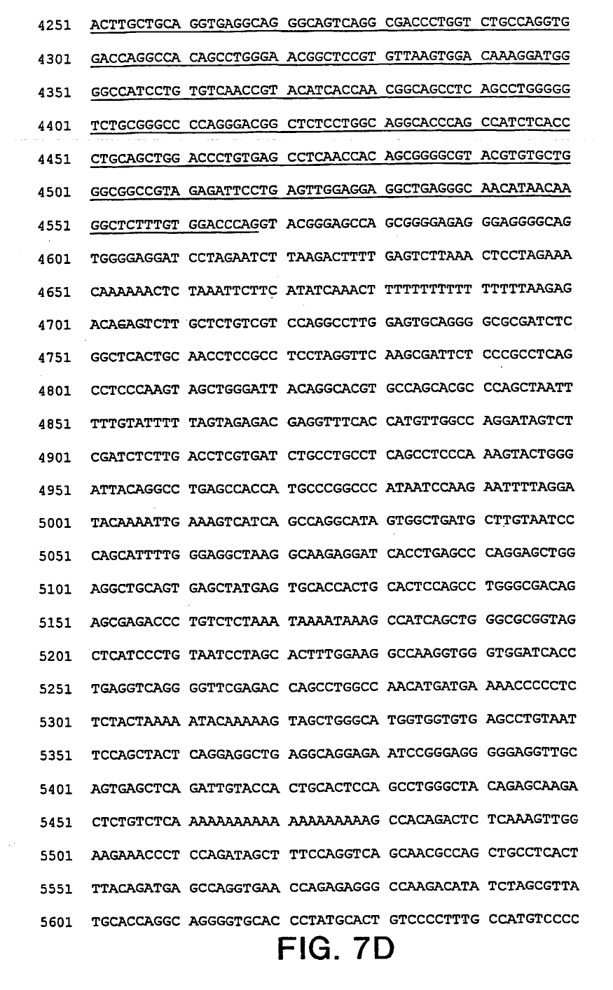

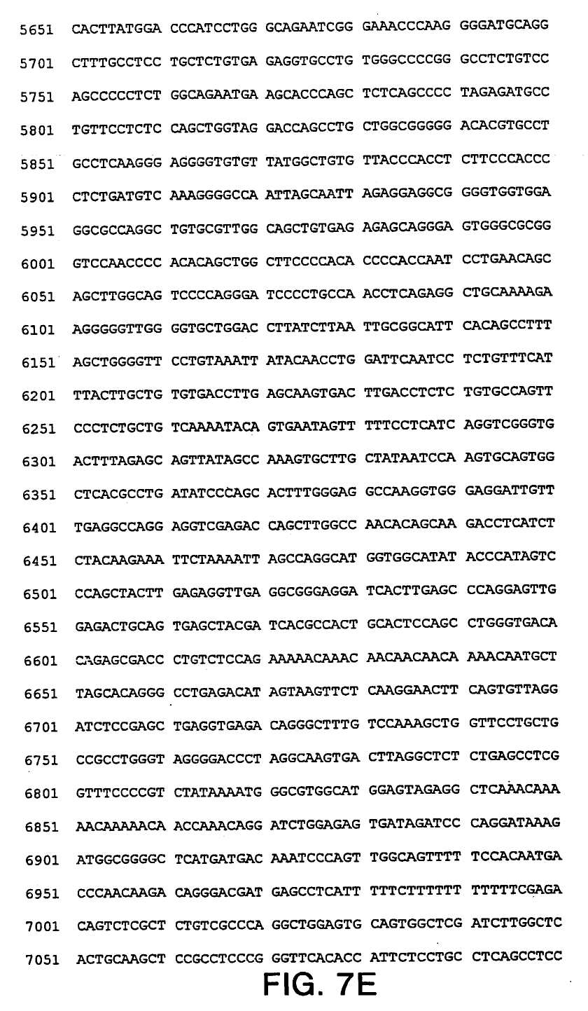

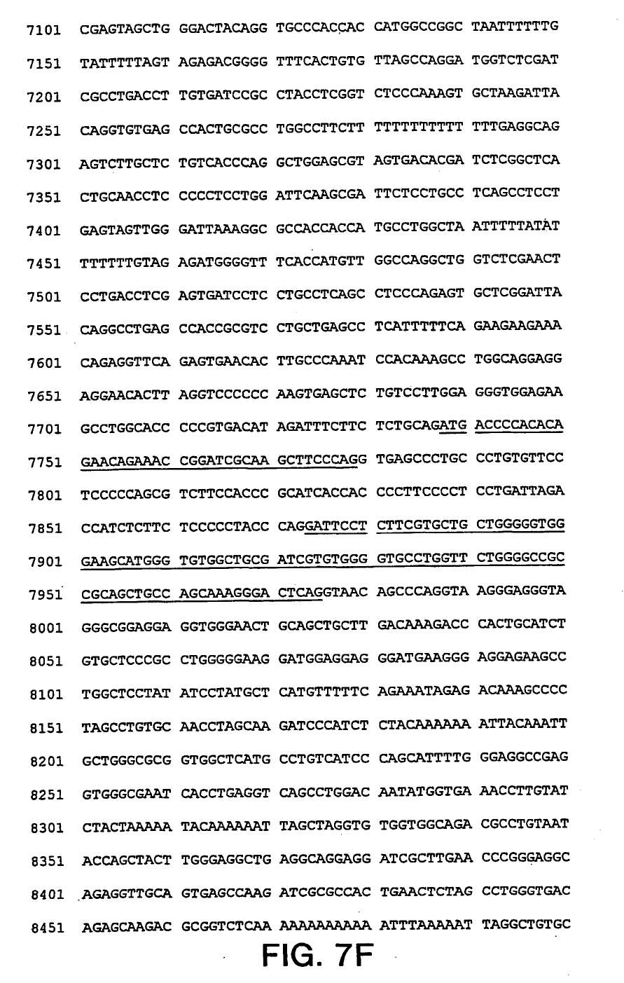

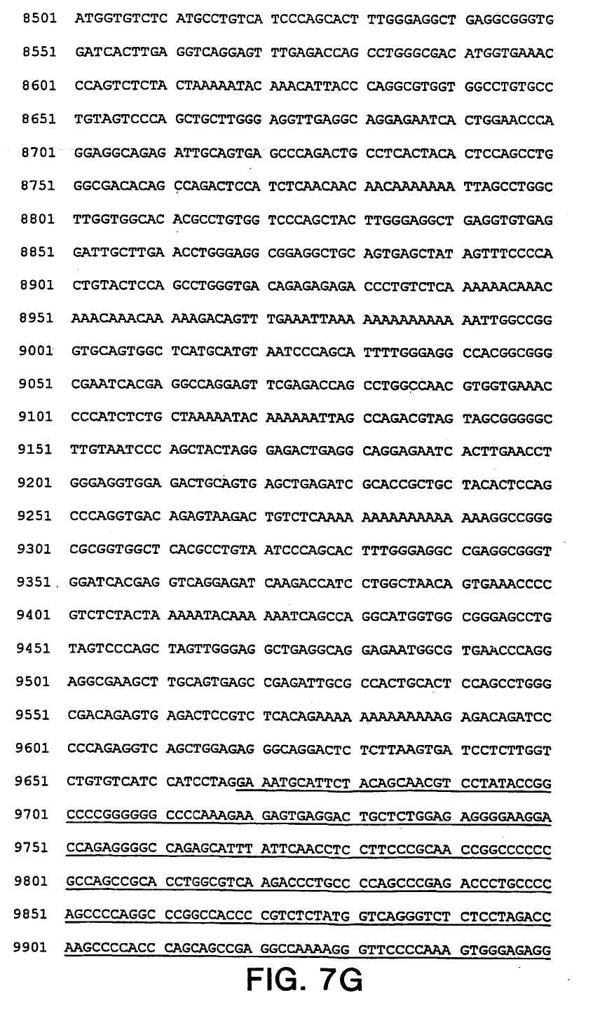

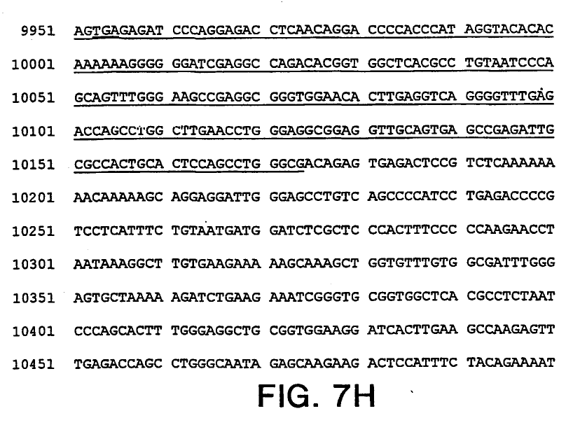

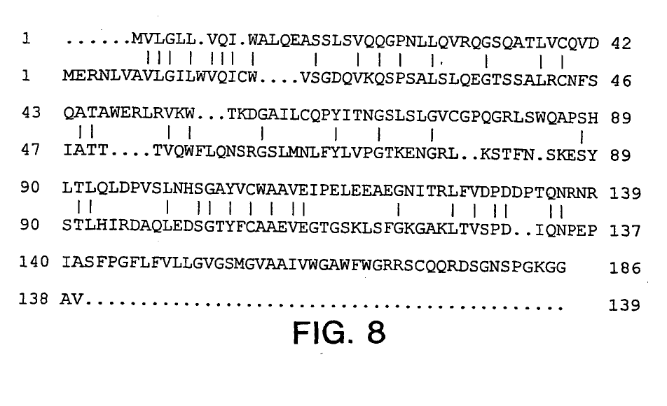

A band of approximately 1.35 kb is observed in the T cell derived cell line, MOLT-4. None of the other cell lines tested, including the B cell line Raji the hematopoietic cell lines HL-60 and K-562, and the epithelial cell line HeLa, showed expression of gp286 (FIG. 5). This indicates that gp286 is expressed specifically in T cells.

### Example 6

### BLAST Search for Homologous Sequences

A BLAST search of the gp286 sequence against a set of ESTs provided by Incyte Genomics (Palo Alto, Calif.) was performed. This search identified an 1195 base pair EST assembly (or template), with template identification number 230543.1. Template 230543.1 was 99% identical to the gp286 sequence over 541 base pairs:

| | . . . . . | |
|---|---|---|
| gp286 | ............GGGCCAGGAATGGGGTCCCCGGGCATGGTGCTGGGCCT | 38 |
| | \|\| \| \|\|\|\|\|\|\|\|\|\|\|\|\|\|\|\|\|\|\|\|\|\|\|\|\|\|\|\|\|\|\|\|\|\|\|\|\|\| | |
| 230543.1 | gtggGGGTGATGGGCCAGtGAATGGGGTCCCCGGGCATGGTGCTGGGCCT | 50 |
| | . . . . . | |
| gp286 | CCTGGTGCAGATCTGGGCCCTGCAAGAAGCCTCAAGCCTGAGCGTGCAGC | 88 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | ACTGGTGCAGATCTGGGCCCTGCAAGAAGCCTCAAGCCTGAGCGTGCAGC | 100 |
| | . . . . . | |
| gp296 | AGGGGCCCAACTTGCTGCAGGTGAGGCAGGGCAGTCAGGCGACCCTGGTC | 138 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | AGGGGCCCAACTTGCTGCAGGTGAGGCAGGGCAGTCAGGCGACCCTGGTC | 150 |
| | . . . . . | |
| gp286 | TGCCAGGTGGACCAGGCCACAGCCTGGGAACGGCTGGCTCCGTGTTAAGTGGAC | 188 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | TGCCAGGTGGACCAGGCCACAGCCTGGGAACGGCTCCGTGTTAAGTGGAC | 200 |
| | . . . . . | |
| gp286 | AAAGGATGGGGCCATCCTGTGTCAACCGTACATCACCAACGGCAGCCTCA | 238 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | AAAGGATGGGGCCATCCTGTGTCAACCGTACATCACCAACGGCAGCCTCA | 250 |
| | . . . . . | |
| gp286 | GCCTGGGGGTCTGCGGGCCCCAGGGACGGCTCTCCTGGCAGGCACCCAGC | 288 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | GCCTGGGGGTCTGCGGGCCCCAGGGACGGCTCTCCTGGCAGGCACCCAGC | 300 |
| | . . . . . | |
| gp286 | CATCTCACCCTGCAGCTGGACCCTGTGAGCCTCAACCACAGCGGGGCGTA | 338 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | CATCTCACCCTGCAGCTGGACCCTGTGAGCCTCAACCACAGCGGGGCGTA | 350 |
| | . . . . . | |
| gp286 | CGTGTGCTGGGCGGCCGTAGAGATTCCTGAGTTGGAGGAGGCTGAGGGCA | 388 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | CGTGTGCTGGGCGGCCGTAGAGATTCCTGAGTTGGAGGAGGCTGAGGGCA | 400 |
| | . . . . . | |
| gp286 | ACATAACAAGGCTCTTTGTGGACCCAGATGACCCCACACAGAACAGAAC | 438 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | ACATAACAAGGCTCTTTGTGGACCCAGATGACCCCACACAGAACAGAAA | 450 |
| | . . . . . | |
| gp286 | CGGATCGCAAGCTTCCCAGGATTCCTCTTCGTGCTGCTGGGGGTGGGAAG | 488 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | CGGATCGCAAGCTTCCCAGGATTCCTCTTCGTGCTGCTGGGGGTGGGAAG | 500 |
| | . . . . . | |
| gp286 | CATGGGTGTGGCTGCGATCGTGTGGGGTGCCTGGTTCTGGGGCCGCCGCA | 538 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 230543.1 | CATGGGTGTGGCTGCGATCGTGTGGGGTGCCTGGTTCTGGGGCCGCCGCA | 550 |

Since the Incyte template sequence included base pairs whose sequence identity was uncertain (shown in lower case), we obtained from Incyte Genomics a clone for one of the component ESTs from the template. This clone, identified by the number 3942653, was then sequenced in its entirety and is shown in SEQ ID NO: 14.

The DNA sequence of clone 3942653 (SEQ ID NO: 14) is shown in Table 4, along with the deduced amino acid sequence of the encoded protein (SEQ ID NO:15). Comparison of this sequence with the mRNA sequence of gp286 and with the genomic sequence in contig 49 of the BAC with the accession number AC008616 (SEQ ID NO:13) reveals that gp286 and gp286a are probably products of alternative splicing of the same gene. The most significant difference between the mRNAs encoded by the two splice forms is in the 3′ end, which encodes the carboxy terminal cytoplasmic portion of the protein. Where gp286 had a cytoplasmic tail of roughly 26 amino acids, gp286a has a cytoplasmic tail of roughly 113 amino acids. In addition, gp286a has three tyrosine residues in the cytoplasmic tail that are potential sites of protein phosphorylation. Phosphorylation is an important mode of signal transduction, and it is likely that gp286a acts as a receptor. For example, the extracellular portion of the protein, with the Ig domain, may receive a signal from an extracellular molecule and the signal is transmitted through the cytoplasmic domain to elicit a growth response in T cells.

### Example 7

### Genomic Library PCR Screening and Subcloning

Subcloning of the gp286 genomic locus may be accomplished by PCR from a genomic library, or directly from genomic DNA For example, two microliters of a human genomic library (˜10⁸ PFU/ml) (Clontech) are added to 6 ml of an overnight culture of K802 cells (Clontech), and then distributed as 250 ml aliquots into each of 24 microtubes. The microtubes are incubated at 37° C. for 15 min. Seven milliliters of 0.8% agarose is added to each tube, mixed, then poured onto LB agar +10 mM MgSO₄ plates and incubated overnight at 37° C. To each plate 5 ml of SM phage buffer (0.1 M NaCl, 8.1 mM MgSO₄.7H₂O, 50 MM Tris-Cl (pH 7.5), 0.01% gelatin) is added and the top agarose is removed with a microscope slide and placed in a 50 ml centrifuge tube. A drop of chloroform is added and the tube is placed in a 37° C. shaker for 15 mill, then centrifuged for 20 min at 4000 rpm (Sorvall RT6000 table top centrifuge) and the supernatant stored at 4° C. as a stock solution.

PCR may be then performed in 20 ml containing 8.8 ml H₂O, 4 ml 5×RAPID-LOAD BUFFER (Origene), 2 ml 10×PCR BLUFFER II (Perkin Elmer), 2 ml 25 mM MgCl₂, 0.8 ml 10 mM dNTP, 0.12 ml of a primer comprising at least a portion of the sequence of the 5′ end of the gp286 polynucleotide of SEQ ID NO: 1 or SEQ ID NO:14 (1 mg/ml), 0.12 ml of a primer comprising at least a portion of the sequence that is complementary to the 3′ end of the gp286 polynucleotide of SEQ ID NO:11 (mg/ml), 0.2 ml AMPLITAQ GOLD polymerase (Perkin Elmer) and 2 ml of phage solution from each of the 24 tubes. The PCR reaction involves 1 cycle at 80° C. for 20 min, 95° C. for 10 min, then 22 cycles at 95° C. for 30 sec, 72° C. for 4 min decreasing 1° C. each cycle, 68° C. for 2 min, followed by 30 cycles at 95° C. for 30 sec, 55° C. for 30 sec, 68° C. for 60 sec. The reaction is loaded onto a 2% agarose gel.

From the tube that gives a PCR product of the correct size, 5 μl is used to set up five 1:10 dilutions that are plated onto LB agar +10 mM MgSO₄ plates and incubated overnight. A BA85 nitrocellulose filter (Schleicher & Schuell) is placed on top of each plate for 1 hour. The filter is removed, placed with the phage side up in a petri dish, and covered with 4 ml of SM buffer for 15 min to elute the phage. One milliliter of SM buffer is removed from each plate and used to set up a PCR reaction as described above. The plate of the lowest dilution to give a PCR product is subdivided, filter-lifted and the PCR reaction is repeated. The series of dilutions and subdividions of the plate is continued until a single plaque is isolated that gives a positive PCR band. Once a single plaque is isolated, 10 ml phage supernatant is added to 100 ml SM and 200 ml of K802 cells per plate with a total of 8 plates set up. The plates are incubated overnight at 37° C. Eight milliliters of SM is added to each plate, and the top agarose is scraped off with a microscope slide and collected in a centrifuge tube.

Three drops of chloroform are added to the centrifuge tube. Subsequently, the tube is vortexed, incubated at 37° C. for 15 min, and centrifuged for 20 min at 4000 rpm (Sorvall RT6000 table top centrifuge) to recover the phage. The recovered phage is used to isolate genomic phage DNA using the QIAGEN LAMBDA MIDI KIT. The sequences for primers may be derived from the sequences given herein.

To subclone the coding region of the gp286 gene, PCR is performed in a 50 μl reaction containing 33 μl H₂O, 5 μl 10×TT buffer (140 mM ammonium sulfate, 0.1% gelatin, 0.6 M Tris-tricine pH 8.4), 5 μl 15 mM MgSO₄, 2 μl 10 mM dNTP, 4 μL genomic phage DNA (0.1 μg/ml), 0.3 μl of a primer comprising at least a portion of the 5′ most coding sequence of the gp286 polynucleotide of SEQ ID NO:1 (1 μg/ml), 0.3 μL of a primer comprising a sequence that is complementary to at least a portion of the 3′ most coding sequence of the gp286 polynucleotide of SEQ ID NO:1(1 μg/ml), 0.4 μl HIGH FIDELITY Taq polymerase (Boehringer Mannheim). The PCR reaction is started with 1 cycle of 94° C. for 2 min followed by 15 cycles at 94° C. for 30 sec, 55° C. for 60 sec., and 68° C. for 2 min.

The PCR product is loaded onto a 2% agarose gel. The DNA band of expected size is excised from the gel, placed in GENELUTE AGAROSE spin column (Supelco) and spun for 10 min at maximum speed. The eluted DNA is ethanol-precipitated and resuspended in 12 μl H₂O for ligation. The PCR primer sequences may be derived from the sequences provided herein.

The ligation reaction uses solutions from the TOPO TA Cloning Kit (Invitrogen). The reaction proceeds in a solution containing 4 μl of PCR product and 1 μL of pCRII-TOPO vector at room temperature for 5 min. The reaction is terminated by the addition of 1 μl of 6×TOPO Cloning Stop Solution. The ligation product is then placed on ice. Two microliters of the ligation reaction is used to transform ONE-SHOT TOP10 cells (Invitrogen). Briefly, the ligation reaction is mixed with the cells and placed on ice for 30 min. The cells are then heat-shocked for 30 seconds at 42° C. and placed on ice for two minutes. Next, 250 μl of SOC is added to the cells, which are incubated at 37° C. with shaking for one hour and then plated onto ampicillin plates.

A single colony from the plates is used to inoculate a 5 ml culture of LB medium. Plasmid DNA is purified from the culture using the CONCERT RAPID PLASMID MINIPREP SYSTEM (GibcoBRL) and the insert of the plasmid DNA is then sequenced.

The gp286 genomic phage DNA may be sequenced using the ABI PRISM 310 Genetic Analyzer (PE Applied Biosystems), which uses the advanced capillary electrophoresis technology and the ABI PRISM BIGDYE Terminator Cycle Sequencing Ready Reaction Kit. The cycle-sequencing reaction may contain 14 ml of H₂O, 16 ml of BIGDYE Terminator mix, 7 ml genomic phage DNA (0.1 mg/ml), and 3 ml primer (25 ng/ml). The reaction is performed in a Perkin-Elmer 9600 thermocycler at 95° C. for 5 min, followed by 99 cycles of 95° C. for 30 sec, 55° C. for 20 sec, and 60° C. for 4 min. The product is purified using a CE X gel filtration cartridge, dried under vacuum, and then dissolved in 16 μl of Template Suppression Reagent (PE Applied Biosystems). The samples are heated at 95° C. for 5 min and then placed in the 310 Genetic Analyzer.

The DNA subcloned into pCRII is sequenced using the ABI PRISM 310 Genetic Analyzer, supra. Each cycle-sequencing reaction contains 6 ml of H₂O, 8 ml of BIGDYE Terminator mix, 5 ml of miniprep DNA (0.1 mg/ml), and 1 ml of primer (25 ng/ml) and is performed in a Perkin-Elmer 9600 thermocycler with 25 cycles of 96° C. for 10 sec, 50° C. for 10 sec, and 60° C. for 4 min. The product is purified using a CENTRIFEX gel filtration cartridge, dried under vacuum, and then dissolved in 16 μl of Template Suppression Reagent. The samples are heated at 95° C. for 5 min and then placed in the 310 Genetic Analyzer.

### Example 7

### GP286 Expression in Tissue by Hybridization Analysis

The expression of gp286 in mammals, such as rat, may be investigated by in situ hybridization histochemistry. To investigate gp286 expression in the thymus, lymph nodes and/or spleen, for example, rat tissue cryosections (20 μm thick) are prepared using a Reichert-Jung cryostat. In addition, blood samples may be prepared for in situ hybridization histochemistry by preparing blood smears, either with total blood or with lymphocyte, particularly T-lymphocyte, enriched fractions. Methods for preparing blood samples for in situ hybridization are well known in the art. Individual sections are thaw-mounted onto silanized, nuclease-free slides (CEL Associates, Inc., Houston, Tex.), and stored at −80° C. Sections are processed starting with post-fixation in cold 4% paraformaldehyde, rinsed in cold phosphate-buffered saline (PBS), acetylated using acetic anhydride in triethanolamine buffer, and dehydrated through a series of alcohol washes in 70%, 95%, and 100% alcohol at room temperature. Subsequently, sections are delipidated in chloroform, followed by rehydration through successive exposure to 100% and 95% alcohol at room temperature. Microscope slides containing processed cryosections are allowed to air dry prior to hybridization. Other tissues, particularly those that are suspected of exhibiting T cell infiltration, may be assayed in a similar fashion.

A gp286-specific probe may be generated using PCR and sequence information from SEQ ID NO:1, SEQ ID NO:13 or SEQ ID NO:14. Following PCR amplification, the fragment is digested with restriction enzymes and cloned into pBluescript II cleaved with the same enzymes. For production of a probe specific for the sense strand of gp286, a cloned gp286 fragment cloned in pBluescript II may be linearized with a suitable restriction enzyme, which provides a substrate for labeled run-off transcripts (i.e., cRNA riboprobes) using the vector-borne T7 promoter and commercially available T7 RNA polymerase. A probe specific for the antisense strand of gp286 may also be readily prepared using the gp286 clone in pBluescript II by cleaving the recombinant plasmid with a suitable restriction enzyme to generate a linearized substrate for the production of labeled run-off cRNA transcripts using the T3 promoter and cognate polymerase.

As discussed above, one may prepare splice variant-specific probes to determine expression of gp286 compared to gp286a in different cells and tissue types.

The riboprobes may be labeled with [³⁵S]-UTP to yield a specific activity of about 0.40×10⁶ cpm/pmol for antisense riboprobes and about 0.65×10⁶ cpm/pmol for sense-strand riboprobes. Each riboprobe may be subsequently denatured and added (2 pmol/ml) to hybridization buffer which contains 50% formamide, 10% dextran, 0.3 M NaCl, 10 mM Tris (pH 8.0), 1 MM EDTA, 1× Denhardt's Solution, and 10 mM dithiothreitol.

Microscope slides containing sequential rat tissue cryosections, or blood smears, may be independently exposed to 45 μl of hybridization solution per slide and silanized cover slips may be placed over the sections being exposed to hybridization solution. Sections are incubated overnight (e.g., 15-18 hours) at 52° C. to allow hybridization to occur. Equivalent series of cryosections or blood smears are then exposed to sense or antisense gp286specific cRNA riboprobes.

Following the hybridization period, coverslips are washed off the slides in 1×SSC, followed by RNase A treatment by exposing the slides to 20 μg/ml RNase A in a buffer containing 10 mM Tris.HCl (pH 7.4), 0.5 M EDTA, and 0.5 M NaCl for 45 minutes at 37° C. The samples are then subjected to three high-stringency washes in 0.1×SSC at 52° C. for 20 minutes each. Following the series of washes, the samples are dehydrated by consecutive exposure to 70%, 95%, and 100% ammonium acetate in alcohol, followed by air drying and exposure to KODAK BIOMAX MR-1 film. After 13 days of exposure, the film is developed, and any significant hybridization signal is detected.

Based on these results, slides containing tissue that hybridized, as shown by film autoradiograms, are coated with KODAK NTB-2 nuclear track emulsion and the slides are stored in the dark for 32 days. The slides are then developed and counterstained with hematoxylin. Emulsion-coated sections are analyzed microscopically to determine the specificity of labeling. The signal is determined to be specific if autoradiographic grains (generated by antisense probe hybridization) are clearly associated with cresyl violate-stained cell bodies.

Autoradiographic grains found between cell bodies indicate non-specific binding of the probe.

### Example 8

### Northern Blot Analysis of gp286-RNA

Northern blot hybridizations may be performed to examine the expression of gp286 mRNA A clone containing at least a portion of the sequence of SEQ ID NO:1, SEQ ID NO:14, or a complement thereto, may be used as a probe. Vector-specific primers are used in PCR to generate a hybridization probe fragment for ³²P-labeling. The PCR is performed as follows:

(1) Mix the Following Reagents:

| | | |
|---|---|---|
| 1 | μl | gp286-containing plasmid |
| 2 | μl | forward primer |
| 2 | μl | reverse primer |
| 10 | μl | 10X PCR buffer provided by the manufacturer of the Taq |
| | | polymerase (e.g., Amersham Pharmacia Biotech) |
| 1 | μl | 10 mM dNTP (e.g., Boebringer Mannheini catalogue no. |
| | | 1 969 064) |
| 0.5 | μl | Taq polymerase (such as Amersham Pharmacia Biotech |
| | | catalogue no. 27-0799-62) |
| 83.5 | μl | water |

(2) Perform PCR in a Thermocylcer Using the Following Program:

94° C., 5 min; 30 cycles of 94° C., 1 min, 55° C., 1 min, and 72° C., 1 min; and then 72° C., 10 min.

The PCR product may be purified using QIAQUICK PCR Purification Kit (Qiagen catalogue no. 28104). The purified PCR fragment is labeled with ³²P-dCT? (Amersham Pharmacia Biotech catalogue no. AA0005/250) by random priming using “Ready-to-go DNA Labeling Beads” (Amersham Pharmacia Biotech cat. no. 27-9240-01). Hybridization is carried out on a human multi-tissue Northern blot from Clonitech according to the manufacturer's protocol.

After overnight exposure on a Molecular Dynamics PHOSPHORIMAGER screen (cat. no. MD146-814), bands of about 1.35 kb are visualized. As discussed previously, one may use a hybridization probe that detects both gp286 and gp286a mRNAs, or may use variant-specific hybridization probes that specifically hybridize to either gp286 or gp286a mRNAs.

### Example 9

### Recombinant Expression of GP286 in Eukaryotic Host Cells

A. Expression of gp286 in Mammalian Cells

To produce GP286 protein, a GP286-encoding polynucleotide is expressed using recombinant techniques. For example, the GP286-encoding sequence of SEQ ID NOs:2 or 15 is subcloned into the commercial expression vector pzeoSV2 (Invitrogen). The resultant expression construct is transfected into Chinese Hamster Ovary (CHO) cells using the transfection reagent FUGENE6 (Boehringer-Mannheim) and the transfection protocol provided in the product insert. Other eukaryotic cell lines, including human embryonic kidney (HEK 293) and COS cells, are suitable as well.

Cells stably expressing GP286 are selected by growth in the presence of 100 μg/ml zeocin (Stratagene, LaJolla, Calif.). Optionally, GP286 may be purified from the cells using standard chromatographic techniques. To facilitate purification, antisera are raised against one or more synthetic peptide sequences that correspond to portions of the GP286 amino acid sequence, and the antisera are used to affinity-purify GP286. The GP286 protein also may be expressed in-frame with a tag sequence (e.g., polyhistidine, haemagglutinin, or FLAG) to facilitate purification. Moreover, it will be appreciated that many of the uses for GP286 polypeptides, such as assays described below, do not require purification of GP286 from the host cell.

B. Expression of GP286 in 293 Cells

For expression of GP286 in mammalian cells 293 (transformed human or primate embryonic kidney cells), a plasmid bearing the relevant gp286 coding sequence is prepared, using vector pSecTag2A (Invitrogen). Vector pSecTag2A contains the murine IgK chain leader sequence for secretion, the c-myc epitope for detection of the recombinant protein with the anti-myc antibody, a C-terminal polyhistidine for purification with nickel chelate chromatography, and a Zeocin-resistant gene for selection of stable transfectants. The forward primer for amplification of this gp286 cDNA is determined by routine procedures and preferably contains a 5′ extension of nucleotides to introduce the HindIII cloning site and nucleotides matching the gp286 sequence. The reverse primer is also determined by routine procedures and preferably contains a 5′ extension of nucleotides to introduce an XhoI restriction site for cloning and nucleotides corresponding to the reverse complement of the gp286 sequence. The PCR conditions are 55° C. as the annealing temperature. The PCR product is gel purified and cloned into the HindIII-XhoI sites of the vector. The DNA is purified using QIAGEN chromatography columns and transfected into 293 cells using the DOTAP transfection medium (Boehringer Mannheim). Transiently transfected cells are tested for expression at 24 hours after transfection, using Western blots probed with anti-His and anti-GP286 peptide antibodies.

Permanently transfected cells are selected with Zeocin and propagated. Production of the recombinant protein is detected from both cells and media by Western blots probed with anti-His, anti-Myc or anti-GP286 peptide antibodies.

C. Expression of GP286 in COS Cells

For expression of GP286 in COS7 cells, a polynucleotide having a sequence of SEQ ID NOs:1 or 14, or a polynucleotide encoding the amino acid sequence of SEQ ID NOs:2 or 15, for example, can be cloned into vector p3-CI. This vector is a pUC18-derived plasmid that contains the HCMV (human cytomegalovirus) promoter-intron located upstream from the bGH (bovine growth hormone) polyadenylation sequence and a multiple cloning site. In addition, the plasmid contains the dhrf (dihydrofolate reductase) gene which provides selection in the presence of the drug methotrexane (MTX) for selection of stable transformants.

The forward primer is determined by routine procedures and preferably contains a 5′ extension which introduces an XbaI restriction site for cloning, followed by nucleotides which correspond to a nucleotide sequence of SEQ ID NOs:1 or 14. The reverse primer is also determined by routine procedures and preferably contains 5′-extension of nucleotides which introduces a SalI cloning site followed by nucleotides which correspond to the reverse complement of a nucleotide sequence of SEQ ID NOs:1 or 14.

The PCR consists of an initial denaturation step of 5 min at 95° C.; 30 cycles of 30 sec denaturation at 95° C., 30 sec annealing at 58° C. and 30 sec extension at 72° C.; and followed by 5 min extension at 72° C. The PCR product is gel purified and ligated into the XbaI and SalI sites of vector p3-CI. This construct is used to transform competent E. coli cells. The plasmid DNA is then purified from the E. coli culture with QIAGEN chromatography columns and transfected into COS7 cells using the LIPOFECTAME reagent from BRL in accordance with the manufacturer's specification. Forty—eight and 72 hours after transfection, the media and the cells are tested for recombinant protein expression.

GP286 expressed from a COS cell culture can be purified by first concentrating the cell-growth media to about 10 mg protein/ml. The purification can be accomplished by, for example, chromatography.

Purified GP286 is concentrated to 0.5 mg/ml in an AMICON concentrator fitted with a YM-10 membrane and stored at −80° C.

D. Expression of GP286 in Insect Cells

For expression of GP286 in a baculovirus system, a polynucleotide having a sequence of SEQ ID NOs:1 or 14, or a polynucleotide encoding an amino acid sequence of SEQ ID NOs:2 or 15, is amplified by PCR The forward primer is determined by routine procedures and preferably contains a 5′ extension which adds the NdeI cloning site, followed by nucleotides which correspond to a nucleotide sequence of SEQ ID NOs:1 or 14. The reverse primer is also determined by routine procedures and preferably contains a 5′ extension which introduces the KpnI cloning site, followed by nucleotides which correspond to the reverse complement of a nucleotide sequence of SEQ ID NOs:1 or 14.

The PCR product is gel purified, digested with NdeI and KpnI, and cloned into the corresponding sites of expression vector pAcHTL-A (Pharmingen, San Diego, Calif.). The pAcHIL vector contains the strong polyhedrin promoter of the Autographa californica nuclear polyhedrosis virus (AcNPMV), and a 6×His tag upstream from the multiple cloning site. Nucleic acid sequences encoding a protein kinase site for phosphorylation and a thrombin site for excision of the recombinant protein precede the multiple cloning site.

Of course, many other baculovirus vectors, such as pAc373, pVL941 and pAcIM1, can be used in place of pAcHTL-A Other suitable vectors for the expression of GP286 polypeptides can be also used, provided that the vector construct includes appropriately located signals for transcription, translation, and trafficking, such as an in-frame AUG and a signal peptide, as required. Such vectors are described in, e.g., Luckow et al., Virology 170:31-39 (1989).

The virus is grown and isolated using standard baculovirus expression methods, such as those described in Summers et al., A MANUAL OF METHODS FOR BACULOVIRUS VECTORS AND INSECT CELL CULTURE PROCEDURES, Texas Agricultural Experimental Station Bulletin No. 1555 (1987). In preferred embodiments, pAcHLT-A containing the gp286 gene is introduced into baculovirus using the BACULOGOLD transfection kit (Pharmingen). Individual virus isolates are analyzed for protein production by radiolabeling infected cells with ³⁵S-methionine at 24 hours post infection. Infected cells are harvested at 48 hours post infection, and the labeled proteins are visualized by SDS-PAGE. Viruses exhibiting high expression levels can be isolated and used for scaled up expression.

For expression of a GP286 polypeptide in a Sf9 cells, a polynucleotide having the sequence of SEQ ID NO:1 can be amplified by PCR using the methods described above for baculovirus expression. The gp286 cDNA is cloned into vector pAcHLT-A (Pharmingen) for expression in Sf9 insect cells. The insert is cloned into the NdeI and KpnI sites, after elimination of an internal NdeI site (using the same primers described above for expression in baculovirus). DNA is purified with QIAGEN chromatography columns and expressed in Sf9 cells. Preliminary Western blot experiments from non-purified plaques are tested for the presence of a recombinant protein of the expected size using a GP286-specific antibody. The results are confirmed after further purification and expression optimization in HiG5 cells.

### Example 10

### Interaction Trap/Two-Hybrid System

In order to assay for GP286-interacting proteins, the interaction trap/two-hybrid library screening method can be used. This assay was first described in Fields et al., Nature 340:245 (1989). A protocol is published in Ausubel et al., SHORT PROTOCOLS IN MOLECULAR BIOLOGY, John Wiley & Sons, NY (1999) and Ausubel, F. M. et al. CURRENT PROTOCOLS IN MOLECULAR BIOLOGY, fourth edition, Greene and Wiley-interscience, NY (1992). Kits are commercially available from, e.g., Clontech (MATCHMAKER Two-Hybrid System 3).

A fusion of the nucleotide sequences encoding all or partial GP286 and the DNA-binding domain (DNA-BD) of yeast transcription factor GAL4 is constructed using an appropriate vector (i.e., pGBKT7). Preferably, fusions are produced for both GP286 and GP286a. Similarly, a GAL4 active domain (AD) fusion library is constructed in a second plasmid (i.e., pGADT7) from cDNA of potential GP286-binding proteins. For protocols on making cDNA libraries, see, e.g., Sambrook et al. MOLECULAR CLONING: A LABORATORY MANUAL, second edition, Cold Spring Harbor Press, Cold Spring Harbor, N.Y. (1989).

The DNA-BD/GP286 fusion construct is verified by sequencing, and tested for autonomous reporter gene activation and cell toxicity, both of which would prevent a successful two-hybrid analysis. Similar controls are performed with the AD/library fusion construct to ensure expression in host cells and lack of transcriptional activity. Yeast cells are transformed (ca. 105 transformants/mg of DNA) with both the GP286 and library fusion plasmids according to standard procedure (Ausubel, et al., supra). In vivo binding of DNA-BD/GP286 with AD/library proteins results in transcription of specific yeast plasmid reporter genes (i.e., lacZ, HIS3, ADE2, LEU2). Yeast cells are plated on nutrient-deficient media to screen for expression of reporter genes. Colonies are dually assayed for b-galactosidase activity upon growth in Xgal (5-bromo-4-chloro-3-indolyl-b-D-galactoside) supplemented media (filter assay for β-galactosidase activity is described in Breeden et al., Cold Spring Harb. Symp. Quant. Biol., 50:643 (1985). Positive AD-library plasmids are rescued from transformants and reintroduced into the original yeast strain as well as other strains containing unrelated DNA-BD fusion proteins to confirm specific GP286/library protein interactions. Insert DNA is sequenced to verify the presence of an open reading frame fused to GAL4 AD and to determine the identity of the GP286-binding protein.

### Example 11

### Antibodies To GP286 Polypeptides

Standard techniques are employed to generate polyclonal or monoclonal antibodies to GP286, and to generate useful antigen-binding fragments thereof or variants thereof, including “humanized” variants and fully human antibodies. Such protocols can be found, for example, in Ausubel et al., 1992 and 1999, supra, Harlow et al. Eds.), ANTIBODIES, A LABORATORY MANUAL, Cold Spring Harbor Laboratory, Cold Spring Harbor, N.Y. (1990) and Harlow et al. (Eds.), ANTIBODIES, A LABORATORY MANUAL, 2d Ed., Cold Spring Harbor Laboratory, Cold Spring Harbor, N.Y. (1999). In some embodiments, recombinant GP286 polypeptides (or cells or cell membranes containing such polypeptides) are used as antigen to generate the antibodies. In some embodiments, one or more peptides having amino acid sequences corresponding to an immunogenic portion of GP286 (e.g., 6, 7, 8, 9, 10, 11, 12, 13, 14, 15, 16, 17, 18, 19, 20, or more amino acids) are used as antigen. Peptides corresponding to extracellular portions of GP286, especially hydrophilic extracellular portions, are preferred. The antigen may be mixed with an adjuvant or linked to a hapten to increase antibody production. In preferred embodiments, antibodies that are specific for the two splice variants, GP286 and GP286a, are prepared by using splice-variant specific amino acid sequences, e.g., using those parts of the intracellular domains that have different amino acid sequences.

A. Polyclonal or Monoclonal Antibodies

In one exemplary protocol, recombinant GP286 or a synthetic fragment thereof is used to immunize a mouse to generate monoclonal antibodies, or to immunize a larger mammal, such as a rabbit, for polyclonal antibodies. The antibodies may be designed to be a “pan-specific” antibody that recognizes both splice variants, GP286 and GP286a, or may be an antibody that recognizes only one of the splice variants. To increase antigenicity, peptides can be conjugated to keyhole limpet hemocyanin commercially available from, e.g., Pierce. For an initial injection, the antigen is emulsified with Freund's Complete Adjuvant and injected subcutaneously. At intervals of two to three weeks, additional aliquots of GP286 antigen are emulsified with Freund's Incomplete Adjuvant and injected subcutaneously. Prior to the final booster injection, a serum sample is taken from the immunized mice and assayed by Western blot to confirm the presence of antibodies that immunoreact with GP286. Sera from the immunized animals may be used as polyclonal antisera or used to isolate polyclonal antibodies that recognize GP286.

Alternatively, the mice are sacrificed and their spleen removed for generation of monoclonal antibodies. To generate monoclonal antibodies, the spleens are placed in 10 ml of serum-free RPMI 1640, and single cell suspensions are formed by grinding the spleens in serum-free RPMI 1640 supplemented with 2 mM 1-glutamine, 1 mM sodium pyruvate, 100 units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a feeder layer. NS-1 myeloma cells, kept in log phase in RPMI with 10% fetal bovine serum (FBS) (Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) is stirred into the pellet, followed by the addition of serum-free RPMI. Thereafter, the cells are centrifuged, resuspended in RPMI containing 15% FBS, 100 μM sodium hypoxanthine, 0.4 μM aminopterin, 16 μM thymidine (HAT) (Gibco), 25 units/ml IL-6 (Boehringer-Mannheim) and 1.5×10⁶ thymocytes/ml, and plated into 10 flat-bottom 96-well tissue culture plates.

On days 2, 4, and 6 after the fusion, 100 pi of medium is removed from the wells of the tissue culture plates and replaced with fresh medium. On day 8, the fusions are screened by ELISA, testing for the presence of mouse IgG that binds to GP286. Cells from selected wells are further cloned by dilution until monoclonal cultures producing anti-GP286 antibodies are obtained.

B. Humanization of Anti-GP286 Monoclonal Antibodies

The expression pattern of GP286 as reported herein and the potential of GP286 as targets for therapeutic intervention suggest therapeutic indications for GP286 inhibitors (antagonists). GP286-neutralizing antibodies comprise one class of therapeutics useful as GP286 antagonists. The following are protocols to improve the utility of anti-GP286 monoclonal antibodies as therapeutics in humans by “humanizing” the monoclonal antibodies. Humanized and fully human antibodies have improved serum half-life and are less immunogenic in humans. The principles of antibody humanization have been described in the literature. For instance, to minimize potential binding to complement, a humanized and fully human antibodies are preferred to be of the IgG₄ subtype.

One level of humanization can be achieved by generating chimeric antibodies comprising the variable domains of a non-human antibody of interest and the constant domains of a human antibody. See, e.g., Morrison et al., Adv. Immunol., 44:65-92 (1989). The variable domains of anti-GP286 antibodies can be cloned from the genomic DNA of an appropriate B-cell hybridoma or from cDNA derived from the hybridoma. The V region gene fragments are linked to exons encoding human antibody constant domains. The resultant construct is expressed in suitable mammalian host cells (e.g., myeloma or CHO cells).

To achieve an even greater level of humanization, only those portions of the variable region gene fragments that encode antigen-binding complementarity determining regions (CDRs) of the non-human monoclonal antibody are cloned into human antibody sequences. See, e.g., Jones et al., Nature 321:522-525 (1986); Riechmann et al., Nature 332:323-327 (1988); Verhoeyen et al., Science 239:1534-36 (1988); and Tempest et al., Bio/Technology 9:266-71 (1991). If necessary, the β-sheet framework of the human antibody surrounding the CDR3 region is also modified (i.e., “back-mutated”) to more closely mirror the three dimensional structure of the antigen-binding site of the original monoclonal antibody. See Kettleborough et al., Protein Engin. 4:773-783 (1991); and Foote et al., J. Mol. Biol. 224:487499 (1992).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Mol. Immunol., 28(4/5):489-98 (1991).

The foregoing approaches are employed using anti-GP286 monoclonal antibodies and the hybridomas that produce them. The humanized anti-GP286 antibodies are useful as therapeutics to treat or palliate conditions wherein GP286 expression or ligand-mediated GP286 signaling is undesirable.

C. Human GP286-Neutralizing Antibodies from Phage Display

Anti-GP286 antibodies can be also generated by phage display techniques such as those described in Aujame et al., Human Antibodies 8(4):155-168 (1997); Hoogenboom, TIBTECH 15:62-70 (1997); and Rader et al., Curr. Opin. Biotechnol. 8:503-508 (1997). For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is screened for GP286-specific phage-antibodies using labeled or immobilized GP286 as antigen-probe.

D. Human GP286-Specific Antibodies from Transgenic Mice

Human GP286-specific antibodies are generated in transgenic mice essentially as described in Brüggemann et al., Immunol. Today 17(8):391-97 (1996) and Brüggemann et al., Curr. Opin. Biotechnol. 8:455-58 (1997). Transgenic mice carrying human V-gene segments in germline configuration and that express these transgenes in their lymphoid tissue are immunized with a GP286 composition using conventional immunization protocols. Hybridomas are generated using B cells from the immunized mice using conventional protocols and screened to identify hybridomas secreting anti-GP286 human antibodies (e.g., as described above).

### Example 12

### Assays to Identify Modulators of GP286 Activity

Set forth below are several non-limiting assays for identifying modulators (agonists and antagonists) of GP286 activity. The assays may be performed on only one of the splice variants of GP286, but in preferred embodiments, the assays are performed in parallel on both splice variants of GP286—GP286 and GP286a. Among the modulators that can be identified by these assays are natural ligands of the receptor; synthetic analogs and derivatives of the natural ligands; antibodies and/or antibody-like compounds derived from natural antibodies or from antibody-like combinatorial libraries; and/or synthetic compounds identified by high-throughput screening of libraries; and the like.

All modulators that bind GP286 are useful for identifying GP286 in tissue samples (e.g., for diagnostic purposes or therapeutic purposes). Agonist and antagonist modulators are useful for up-regulating and down-regulating GP286 activity, respectively, so as to treat GP286-mediated diseases. The assays may be performed using single putative modulators, and/or may be performed using a known agonist in combination with candidate antagonists (or visa versa).

A. cAMP Assays

In one type of assay, levels of cyclic adenosine monophosphate (cAMP) are measured in gp286-transfected cells that have been exposed to candidate modulator compounds. Protocols for cAMP assays have been described in the literature. See, e.g., Sutherland et al., Circulation 37:279 (1968); Frandsen et al., Life Sciences 18:529-541 (1976); Dooley et al., J. of Pharmacol. Exp. Therap. 283(2): 73541 (1997); and George et al., J. of Biomol. Screening 2(4):235-40 (1997). An exemplary protocol for such an assay, using an Adenylyl Cyclase Activation FLASHPLATE Assay from NEN Life Science Products, is set forth below.

Briefly, a GP286-encoding sequence is subcloned into an expression vector, such as pzeoSV2 (Invitrogen). CHO cells are transiently transfected with the resultant expression construct using known methods, such as the transfection protocol provided by Boehringer-Mannheim when supplying the FUGENE 6 transfection reagent. Transfected CHO cells are seeded into 96-well microplates from the FLASHPLATE assay kit, which are coated with solid scintillant to which antisera to cAMP have been bound. For a control, some wells are seeded with untransfected CHO cells. Other wells in the plate receive various amounts of a cAMP standard solution for use in creating a standard curve. One or more test compounds are added to the cells in each well, with compound-free medium or buffer as control. After treatment, cAMP is allowed to accumulate in the cells for exactly 15 minutes at room temperature. The assay is terminated by the addition of lysis buffer containing [¹²⁵I]-cAMP, and the plate is counted using a Packard TOPCOUNT 96-well microplate scintillation counter. Unlabeled cAMP from the lysed cells or from standards and fixed amounts of [¹²⁵I]-cAMP compete for antibody bound to the plate. A standard curve is constructed, and cAMP values for the unknowns are obtained by interpolation. Changes in intracellular cAMP levels of cells in response to exposure to a test compound are indicative of GP286 modulating activity. Modulators that act as agonists of receptors which couple to the Gs subtype of G proteins will stimulate production of cAMP, leading to a measurable (e.g., 3-10) fold increase in cAMP levels. Agonists of receptors which couple to the Gi/o subtype of G proteins will inhibit forskolin-stimulated cAMP production, leading to a measurable decrease (e.g., 50-100%) in cAMP levels. Modulators that act as inverse agonists will reverse these effects at receptors that are either constitutively active or activated by known agonists.

B. Aequorin Assays

In another assay, cells (e.g., CHO cells) are transiently co-transfected with a gp286 expression construct and a construct that encodes the photoprotein apoaquorin. In the presence of the cofactor coelenterazine, apoaquorin will emit a measurable luminescence that is proportional to the amount of cytoplasmic free calcium. See generally, Cobbold, et al. “Aequorin measurements of cytoplasmic free calcium,” In: McCormack J. G. and Cobbold P. H., eds., CELLULAR CALCIUM: A PRACTICAL APPROACH. Oxford:IRL Press (1991); Stables et al., Anal. Biochem. 252:115-26 (1997); and Haugland, HANDBOOK OF FLUORESCENT PROBES AND RESEARCH CHEMICALS, Sixth edition, Eugene Oreg. (1996).

In one exemplary assay, a gp286 coding sequence is subcloned into pzeoSV2 (Invitrogen). CHO cells are transiently co-transfected with the resultant expression construct and a construct that encodes the photoprotein apoaquorin (Molecular Probes) using the transfection reagent FUGENE 6 (Boehringer-Mannheim) and the transfection protocol provided in the product insert.

The cells are cultured for 24 hours at 37° C. in MEM (Gibco/BRL, Gaithersburg, Md.) supplemented with 10% fetal bovine serum, 2 mM glutamine, 10 U/ml penicillin and 10 μg/ml streptomycin. Then the culture medium is changed to serum-free MEM containing 5 μM coelenterazine (Molecular Probes). Culturing is continued for two more hours at 37° C. Subsequently, the cells are detached from the plate using VERSEN (Gibco/BRL), washed, and resuspended at 2×10⁵ cells/ml in serum-free MEM.

Dilutions of candidate GP286 modulator compounds are prepared in serum-free MEM and dispensed into wells of an opaque 96-well assay plate at 50 μl/well. The plate is then loaded onto an MLX microtiter plate luminometer (Dynex Technologies, Inc., Chantilly, Va.). The instrument is programmed to dispense 50 μl cell suspensions into each well, one well at a time, and immediately read luminescence for 15 seconds. Dose-response curves for the candidate modulators are constructed using the area under the curve for each light signal peak. Data are analyzed with SLIDEWRITE, using the equation for a one-site ligand, and EC50 values are obtained. Changes in luminescence caused by the compounds are considered indicative of modulatory activity. Modulators that act as agonists at receptors which couple to the Gq subtype of G proteins give an increase in luminescence of up to 100 fold. Modulators that act as inverse agonists will reverse this effect at receptors that are either constitutively active or activated by known agonists.

C. Luciferase Reporter Gene Assay

The photoprotein luciferase provides another useful tool for identifying GP286 modulators. Cells (e.g., CHO cells or COS7 cells) are transiently co-transfected with a gp286 expression construct and a reporter construct which includes a gene for the luciferase protein downstream from a transcription factor binding site, such as the cAMP-response element (CRE), AP-1, or NF-kappa B.

Expression levels of luciferase reflect the activation status of the signaling events. See generally, George et al., J. Biomol. Screening 2(4):235-240 (1997); and Stratowa et al., Curr. Opin. Biotechnol. 6:574-581 (1995). Luciferase activity may be quantitatively measured using, e.g., luciferase assay reagents that are available from Promega (Madison, Wis.).

In one exemplary assay, CHO cells are plated in 24well culture plates at a density of 10⁵ cells/well one day prior to transfection, and cultured at 37° C. in MEM (Gibco/BRL) supplemented with 10% fetal bovine serum, 2 mM glutamine, 10 U/ml penicillin and 10 μg/ml streptomycin. Cells are transiently co-transfected with a gp286 expression construct and a reporter construct containing the luciferase gene. The reporter plasmid constructs CRE-luciferase, AP-1-luciferase and NF-kappaB-luciferase may be purchased from Stratagene (LaJolla, Calif.). Transfections are performed using the FUGENE 6 transfection reagent (Boehringer-Mannheim) according to the supplier's instructions. Cells transfected with the reporter construct alone are used as a control.

Twenty-four hours after transfection, the cells are washed once with PBS pre-warmed to 37° C. Serum-free MEM is then added to the cells either alone (control) or with one or more candidate modulators. The cells are then incubated at 37° C. for five hours. Thereafter, the cells are washed once with ice-cold PBS and lysed by the addition of 100 μl of lysis buffer per well from the luciferase assay kit supplied by Promega. After incubation for 15 minutes at room temperature, 15 μl of the lysate is mixed with 50 μl of substrate solution (Promega) in an opaque-white, 96-well plate, and the luminescence is read immediately on a Wallace model 1450 MICROBETA scintillation and luminescence counter (Wallace Instruments, Gaithersburg, Md.).

Differences in luminescence in the presence versus the absence of a candidate modulator compound are indicative of modulatory activity. Receptors that are either constitutively active or activated by agonists typically give a 3-fold to 20-fold stimulation of luminescence compared to cells transfected with the reporter gene alone. Modulators that act as inverse agonists will reverse this effect.

D. Intracellular Calcium Measurement Using FLIPR

Changes in intracellular calcium levels are another recognized indicator of receptor activity, and such assays can be employed to screen for modulators of GP286 activity. For example, CHO cells stably transfected with a gp286 expression vector are plated at a density of 4×10⁴ cells/well in Packard black-walled, 96-well plates specially designed to discriminate fluorescence signals emanating from the various wells on the plate. The cells are incubated for 60 minutes at 37° C. in modified Dulbecco's PBS (D-PBS) containing 36 mg/L pyruvate and 1 g/L glucose with the addition of 1% fetal bovine serum and one of four calcium indicator dyes (FLUO-3 AM, FLUO-4 AM, CALCIUM GREEN-1 AM, or OREGON GREEN 488 BAPTA-1 AM), each at a concentration of 4 μM. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

A calcium response is initiated by the addition of one or more candidate receptor agonist compounds, calcium ionophore A23187 (10 μM; positive control), or ATP (4 μM; positive control). Fluorescence is measured by Molecular Device's FLIPR with an argon laser (excitation at 488 nm). See, e.g., Kuntzweiler et al., Drug Dev. Res. 44(1):14-20 (1998). The F-stop for the detector camera is set at 2.5 and the length of exposure is 0.4 milliseconds. Basal fluorescence of cells is measured for 20 seconds prior to addition of a candidate agonist, ATP, or A23187. The basal fluorescence level is subtracted from the response signal. The calcium signal is measured for approximately 200 seconds, taking readings every two seconds. Calcium ionophore A23187 and ATP typically increase the calcium signal about 200% above baseline levels. In general, activated GP286s increase the calcium signal at least about 10-15% above baseline signal.

E. Mitogenesis Assay

In a mitogenesis assay, the ability of candidate modulators to induce or inhibit gp286-mediated cell division is determined. See, e.g., Lajiness et al., J. Pharmacol. and Exp. Therap. 267(3):1573-1581 (1993). For example, CHO cells stably expressing GP286 are seeded into 96-well plates at a density of 5000 cells/well and grown at 37° C. in MEM with 10% fetal calf serum for 48 hours, at which time the cells are rinsed twice with serum-free MEM. After rinsing, 80 Ill of fresh MEM, or MEM containing a known mitogen, is added along with 20 μl MEM containing varying concentrations of one or more test compounds diluted in serum-free medium. As controls, some wells on each plate receive serum-free medium alone, and some receive medium containing 10% fetal bovine serum. Untransfected cells or cells transfected with vector alone also may serve as controls.

After culture for 16-18 hours, 1 μCi of [³H]-thymidine (2 Ci/mmol) is added to the wells and cells are incubated for an additional 2 hours at 37° C. The cells are trypsinized and collected on filter mats with a cell harvester (Tomtec); the filters are then counted in a Betaplate counter. The incorporation of [³H]-thymidine in serum-free test wells is compared to the results achieved in cells stimulated with serum (positive control). Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC50; D is the concentration of the compound; and G is the maximal effect. Parameters B, C and G are determined by Simplex optimization.

Agonists that bind to the receptor are expected to increase [³H]-thymidine incorporation into cells, showing up to 80% of the response to serum. Antagonists that bind to the receptor will inhibit the stimulation seen with a known agonist by up to 100%.

F. [³⁵S]GTPgS Binding Assay

It is possible to evaluate whether GP286 signals through a G protein-mediated pathway. G protein-coupled receptors signal through intracellular G proteins whose activities involve GT? binding and hydrolysis to yield bound GDP. Thus, measurement of binding of the non-hydrolyzable GTP analog [³⁵S]GTgS in the presence and absence of candidate modulators provides another assay for modulator activity. See, e.g., Kowal et al., Neuropharmacology 37:179-187 (1998).

In one exemplary assay, cells stably transfected with a gp286 expression vector are grown in 10 cm tissue culture dishes to subconfluence, rinsed once with 5 ml of ice-cold Ca²⁺/M²⁺-free phosphate-buffered saline, and scraped into 5 ml of the same buffer. Cells are pelleted by centrifugation (500×g, 5 minutes), resuspended in TEE buffer (25 mM Tris, pH 7.5, 5 mM EDTA, 5 mM EGTA), and frozen in liquid nitrogen. After thawing, the cells are homogenized using a Dounce homogenizer (1 ml LEE per plate of cells), and centrifuged at 1,000×g for 5 minutes to remove nuclei and unbroken cells.

The homogenate supernatant is centrifuged at 20,000×g for 20 minutes to isolate the membrane fraction, and the membrane pellet is washed once with TEE and resuspended in binding buffer (20 mM HEPES, pH 7.5, 150 mM NaCl, 10 mM MgCl₂, 1 mM EDTA). The resuspended membranes can be frozen in liquid nitrogen and stored at −70° C. until use.

Aliquots of cell membranes prepared as described above and stored at −70° C. are thawed, homogenized, and diluted into buffer containing 20 mM HEPES, 10 mM MgCl2, 1 mM EDTA, 120 mM NaCl, 10 μM GDP, and 0.2 mM ascorbate, at a concentration of 10-50 μg/ml. In a final volume of 90 μl, homogenates are incubated with varying concentrations of candidate modulator compounds or 100 μM GTP for 30 minutes at 30° C. and then placed on ice. To each sample, 10 μl guanosine 5′-O-(3[³⁵S]thio) triphosphate (NEN, 1200 Ci/mmol; [³⁵S]-GTPgS), was added to a final concentration of 100-200 μM. Samples are incubated at 30° C. for an additional 30 minutes, 1 ml of 10 mM HEPES, pH 7.4, 10 mM MgCl2, at 4° C. is added and the reaction is stopped by filtration.

Samples are filtered over Whatman GF/B filters and the filters are washed with 20 ml ice-cold 10 mM HEPES, pH 7.4, 10 mM MgCl₂. Filters are counted by liquid scintillation spectroscopy. Nonspecific binding of [³⁵S]-GTPgS is measured in the presence of 100 μM GTP and subtracted from the total. Compounds are selected that modulate the amount of [³⁵S]-GTPgS binding in the cells, compared to untransfected control cells. Activation of receptors by agonists gives up to a five-fold increase in [³⁵S]-GTPgS binding. This response is blocked by antagonists.

G. MAP Kinase Activity Assay

Evaluation of MAP kinase activity in cells expressing GP286 provides another assay to identify modulators of GP286 activity. See, e.g., Lajiness et al., J. Pharmacol. Exp. Therap. 267(3):1573-1581 (1993) and Boulton et al., Cell 65:663-675 (1991). In some embodiments, CHO cells stably transfected with gp286 are seeded into 6-well plates at a density of 7×10⁴ cells/well 48 hours prior to the assay. During this 48 hour period, the cells are cultured at 37° C. in MEM medium supplemented with 10% fetal bovine serum, 2 mM glutamine, 10 U/ml penicillin and 10 μg/ml streptomycin. The cells are serum-starved for 1-2 hours prior to the addition of stimulants.

For the assay, the cells are treated with medium alone or medium containing either a candidate agonist or 200 nM Phorbol ester-myristoyl acetate (i.e., PMA, a positive control), and the cells are incubated at 37° C. for various amounts of time. To stop the reaction, the plates are placed on ice, the medium is aspirated, and the cells are rinsed with 1 ml of ice-cold PBS containing 1 mM EDTA Thereafter, 200 μL of cell lysis buffer (12.5 mM MOPS, pH 7.3, 12.5 mM glycerophosphate, 7.5 mM MgCl₂, 0.5 mM EGTA, 0.5 mM sodium vanadate, 1 mM benzamidine, 1 mM dithiothreitol, 10 μg/ml leupeptin, 10 μg/ml aprotiin, 2 μg/ml pepstatin A, and 1 μM okadaic acid) is added to the cells. The cells are scraped from the plates and homogenized by 10 passages through a 23¾ G needle, and the cytosol fraction is prepared by centrifugation at 20,000×g for 15 minutes.

Aliquots (5-10 μl containing 1-5 μg protein) of cytosol are mixed with 1 mM MAPK Substrate Peptide (APRTPGGRR (SEQ ID NO:9), Upstate Biotechnology, Inc., NY) and 50 μM [g-3P]ATP (NEN, 3000 Ci/mmol), diluted to a final specific activity of about 2000 cpm/pmol, in a total volume of 25 μl. The samples are incubated for 5 minutes at 30° C., and reactions are stopped by spotting 20 μl on 2 cm² squares of Whatman P81 phosphocellulose paper. The filter squares are washed in 4 changes of 1% H₃PO₄, and the squares are subjected to liquid scintillation spectroscopy to quantitate bound label. Equivalent cytosolic extracts are incubated without MAPK substrate peptide, and the bound labels from these samples are subtracted from the matched samples with the substrate peptide. The cytosolic extract from each well is used as a separate point. Protein concentrations are determined by a dye binding protein assay (Bio-Rad Laboratories). Agonist activation of the receptor is expected to result in up to a five-fold increase in MAPK enzyme activity. This increase is blocked by antagonists.

H. [³H]Arachidonic Acid Release

The activation of GP286s may also potentiate arachidonic acid release in cells, providing yet another useful assay for modulators of GP286 activity. See, e.g., Kanterman et al., Molecular Pharmacology 39:364-369 (1991). For example, CHO cells that are stably transfected with a GP286 expression vector are plated in 24-well plates at a density of 1.5×10⁴ cells/well and grown in MEM medium supplemented with 10% fetal bovine serum, 2 mM glutamine, 10 U/ml penicillin and 10 μg/ml streptomycin for 48 hours at 37° C. before use. Cells of each well are labeled by incubation with [³H]-arachidonic acid (Amersham Corp., 210 Ci/mmol) at 0.5 μCi/ml in 1 ml MEM supplemented with 10 mM HEPES, pH 7.5, and 0.5% fatty-acid-free bovine serum albumin for 2 hours at 37° C. The cells are then washed twice with 1 ml of the same buffer. Candidate compounds are added in 1 ml of the same buffer, either alone or with 10 μM ATP, and the cells are incubated at 37° C. for 30 minutes. Buffer alone and mock-transfected cells are used as controls. Samples (0.5 ml) from each well are counted by liquid scintillation spectroscopy. Agonists which activate the receptor will lead to potentiation of the ATP-stimulated release of [³H]-arachidonic acid. This potentiation is blocked by antagonists.

I. Extracellular Acidification Rate

In yet another assay, the effects of candidate modulators of GP286 activity are assayed by monitoring extracellular changes in pH induced by the test compounds. See, e.g., Dunlop et al., J. Pharmacol. Toxicol. Meth. 40(1):47-55 (1998). In some embodiments, CHO cells transfected with a GP286 expression vector are seeded into 12 mm capsule cups (Molecular Devices Corp.) at 4×10⁵ cells/cup in MEM supplemented with 10% fetal bovine serum, 2 mM L-glutamine, 10 U/ml penicillin, and 10 μg/ml streptomycin. The cells are incubated in this medium at 37° C. in 5% CO₂ for 24 hours.

Extracellular acidification rates are measured using a CYTOSENSOR MICROPHYSIOMETER (Molecular Devices Corp.). The capsule cups are loaded into the sensor chambers of the MICROPHYSIOMETER and the chambers are perfused with running buffer (bicarbonate-free MEM supplemented with 4 mM L-glutamine, 10 units/ml penicillin, 10 μg/ml streptomycin, 26 mM NaCl) at a flow rate of 100 μl/min. Candidate agonists or other agents are diluted into the running buffer and perfused through a second fluid path. During each 60-second pump cycle, the pump is run for 38 seconds and is off for the remaining 22 seconds. The pH of the running buffer in the sensor chamber is recorded during the cycle from 43-58 seconds, and the pump is re-started at 60 seconds to start the next cycle. The rate of acidification of the running buffer during the recording time is calculated by the Cytosoft program. Changes in the rate of acidification are calculated by subtracting the baseline value (the average of 4 rate measurements immediately before addition of a modulator candidate) from the highest rate measurement obtained after addition of a modulator candidate. The selected instrument detects 61 mV/pH unit. Modulators that act as agonists of the receptor result in an increase in the rate of extracellular acidification compared to the rate in the absence of agonist. This response is blocked by modulators which act as antagonists of the receptor.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

What is claimed is:

1. An isolated polynucleotide comprising a nucleotide sequence selected from the group consisting of:
(a) SEQ ID NO: 1, and allelic variants thereof;
(b) a fragment of (a), consisting of at least 17 nucleotides;
(c) a nucleotide sequence that encodes SEQ ID NO:2, an allelic variant thereof or fragments of either consisting of at least six amino acid residues;
(d) a nucleotide sequence that is at least 65% identical to SEQ ID NO: 1;
(e) a nucleotide sequence that encodes a protein that is at least 80% homologous to SEQ ID NO:2;
(f) a nucleotide sequence that hybridizes to SEQ ID NO: 1 under high stringency conditions; and
(g) a complementary sequence of any of (a) through (f);
(h) SEQ ID NO: 14, and allelic variants thereof
(i) a fragment of (h), consisting of at least 17 nucleotides;
(j) a nucleotide sequence that encodes SEQ ID NO: 15, an allelic variant thereof or fragments of either consisting of at least six amino acid residues;
(j) a nucleotide sequence that is at least 65% identical to SEQ ID NO:14;
(l) a nucleotide sequence that encodes a protein that is at least 80% homologous to SEQ ID NO:15;
(m) a nucleotide sequence that hybridizes to SEQ ID NO: 14 under high stringency conditions; and
(n) a complementary sequence of any of (h) through (m).

2. The polynucleotide of claim 1, wherein said polynucleotide comprises a nucleotide sequence selected from SEQ ID NO:1 or SEQ ID NO:14, or an allelic variant of SEQ ID NO: 1 or SEQ ID NO:14.

3. The polynucleotide of claim 1, wherein said polynucleotide comprises a nucleotide sequence selected from any one of SEQ ID NOs: 3, 4, 5, 16, 18, 20, 22, 24, 26, 28, 30 or 32, or an allelic variant of any one of SEQ ID NOs: 3, 4, 5, 16, 18, 20, 22, 24, 26, 28, 30 or 32.

4. The polynucleotide of claim 1, wherein said polynucleotide comprises a nucleotide sequence encoding an amino acid sequence selected from SEQ ID NO:2 or SEQ ID NO:15, or an allelic variant of SEQ ID NO:2 or SEQ BD NO:15.

5. The polynucleotide of claim 1, wherein said polynucleotide comprises a nucleotide sequence encoding an amino acid sequence selected from SEQ ID NOs: 17, 19, 21, 23, 25, 27, 29, 31 or 33, or an allelic variant of SEQ ID NOs: 17, 19, 21, 23, 25, 27, 29, 31 or 33.

6. The polynucleotide of claim 1, further comprising a transcription regulatory sequence operatively linked to any one of (a) through (m).

7. The polynucleotide of claim 1, further comprising a nucleotide sequence encoding a heterologous polypeptide.

8. The polynucleotide of claim 1, comprising a nucleotide sequence that is complementary to any one of (i) SEQ ID NOs:1, 3 or 14 or (ii) a fragment of any one of SEQ ID NOs: 1, 3 or 14 consisting of at least 17 nucleotides.

9. A vector comprising a polynucleotide of claim 1.

10. The vector of claim 9, which is a plasmid vector.

11. The vector of claim 9, which is a viral vector.

12. Vector of claim 11, which is a baculoviral vector.

13. The vector of claim 11, selected from the group consisting of adenoviruses, parvoviruses, herpesviruses, poxyiruses, adeno-associated viruses, Semliki Forest viruses, vaccinia viruses, lentiviruses and retroviruses.

14. A host cell containing the polynucleotide of claim 1.

15. The host cell of claim 14, wherein the host cell is selected from the groups consisting of a bacterial cell, insect cell, yeast cell, plant cell and mammalian cell.

16. The host cell of claim 14, wherein the host cell is a human cell.

17. An isolated polypeptide encoded by the polynucleotide of claim 1.

18. The polypeptide of claim 17, comprising (i) SEQ ID NO:2 or SEQ ID NO: 15, or an allelic variant of SEQ ID NO:2 or SEQ ID NO: 15, or (ii) a fragment of (i) consisting of at least six amino acid residues.

19. The polypeptide of claim 17, comprising SEQ ID NOs: 17, 19, 21, 23, 25, 27, 29, 31 or 33, or an allelic variant of SEQ ID NOs: 17, 19, 21, 23, 25, 27, 29, 31 or 33.

20. The polypeptide of any one of claims 17-19, further comprising a heterologous sequence.

21. A composition comprising the polynucleotide of claim 1 and a pharmaceutically acceptable carrier.

22. A composition comprising the polypeptide of any one of claims 17-19 and a pharmaceutically acceptable carrier.

23. An antibody that binds to an epitope on the polypeptide of any one of claims 17-19.

24. The antibody of claim 23, wherein the antibody is a monoclonal antibody.

25. The antibody of claim 24, wherein the antibody is a humanized or fully human antibody.

26. The antibody of claim 23, which is specific for the gp286 splice variant.

27. The antibody of claim 23, which is specific for the gp286a splice variant.

28. A composition comprising the antibody of any one of claims 23-27 and a pharmaceutically acceptable carrier.

29. A method of producing a polypeptide, comprising the steps of:
culturing the host cell of claim 14 in a medium under conditions in which said polynucleotide is expressed, and
recovering the polypeptide from the cell or from the culture medium.

30. A method of determining the presence of a gp286-encoding sequence in a sample, comprising the steps of:
contacting the sample with the isolated polynucleotide of claim 1 under high stringency hybridization conditions, and
detecting hybridization of said isolated polynucleotide to a polynucleotide in the sample, wherein the occurrence of said hybridization indicates the presence of a gp286-encoding sequence in the sample.

31. The method according to claim 30, wherein said polynucleotide is specific for the gp286 splice variant.

32. The method according to claim 30, wherein said polynucleotide is specific for the gp286a splice variant.

33. A method of determining the presence of a GP286 protein in a sample, comprising the steps of
contacting the sample with the antibody of any one of claim 23-27; and
detecting specific binding of said antibody to an antigen, wherein the occurrence of said specific binding indicates the presence of a GP286 protein in the sample.

34. The method according to claim 33, wherein said antibody is specific for the gp286 splice variant.

35. The method according to claim 33, wherein said antibody is specific for the gp286a splice variant.

36. A method of identifying a compound that binds a GP286 protein, comprising the steps of:
contacting a GP286 protein with a test compound; and
detecting a complex formed by said GP286 protein and said test compound, wherein the presence of said complex indicates that said test compound binds to said GP286 protein.

37. A method of identifying a compound that modulates the activity of a GP286 protein, comprising the steps of:
contacting said GP286 protein with a test compound; and
determining the effect of the test compound on the activity of said GP286 protein, whereas a change of said activity after the contacting step indicates that said test compound modulates the activity of said GP286 protein.

38. A method of identifying a homolog of a human gp286 gene, comprising the steps of screening a nucleotide database with a query sequence consisting of SEQ ID NO:1 or SEQ ID NO:14 or a portion of SEQ ID NO:1 or SEQ ID NO:14 consisting of 300 or more nucleotides,
wherein a nucleotide sequence in said database that is at least 65% but less than 100% identical to SEQ ID NO: 1 or SEQ ID NO: 14 or said portion of SEQ ID NO:1 or SEQ ID NO: 14, if found, is a homolog of a human gp286 gene.

39. A method of identifying a homolog of a human gp286 gene, comprising the steps of:
hybridizing a polynucleotide library with a polynucleotide probe under high stringency hybridization conditions comprising SEQ ID NO:1 or SEQ ID NO:14, or a portion of SEQ ID NO:1 or SEQ ID NO:14 consisting of at least 17 nucleotides; and
determining whether said polynucleotide probe hybridizes to a polynucleotide in the library,
wherein the polynucleotide so hybridized is a homolog of a human gp286 gene.

40. A method of diagnosing a disease condition in a subject, comprising the step of comparing the amount or activity of a GP286 protein in a tissue sample from said subject to the amount or activity of the GP286 polypeptide in a control sample,
wherein a significant difference in the amount or activity of said GP286 polypeptide in said tissue sample relative to the amount or activity of said GP286 polypeptide in said control sample indicates that the subject has a disease condition.

41. The method of claim 40, wherein the disease condition relates to the immune system.

42. The method of claim 41, wherein the disease condition relates to T cells.

43. A method of diagnosing a disease condition in a subject, comprising the step of comparing the amount of a gp286 mRNA in a tissue sample from the subject to the amount of said gp286 mRNA in a control sample,
wherein a significant difference in the amount of said mRNA in said tissue sample relative to the amount of said mRNA in said control sample indicates that the subject has a disease condition.

44. The method of claim 43, wherein the disease condition relates to the immune system.

45. The method of claim 44, wherein the disease condition relates to T cells.

46. A diagnostic assay for identifying in a test cell the presence or absence of a genetic lesion or mutation characterized by at least one of: (i) aberrant modification or mutation of a gene encoding a GP286 protein; (ii) mis-regulation of a gene encoding a GP286 protein; and (iii) aberrant post-translational modification of a GP286 protein; comprising the steps of
separately hybridizing polynucleotides from the test cell and from a reference cell that lacks said genetic lesion or mutation with a polynucleotide probe comprising SEQ ID NO: 1, SEQ ID NO: 14 or a portion thereof consisting of at least 17 nucleotides, under high stringency hybridization conditions; and
separately washing said polynucleotide hybrids under high stringency wash conditions to allow dissociation of the hybrids; and
determining whether said polynucleotide probe dissociates more readily from the polynucleotides of the test cell compared to the polynucleotides of the reference cell.

47. The use of a composition of claims 21 or 22 for the treatment of a disease condition that relates to the immune system.

48. The use of claim 47, wherein the disease condition is selected from the group consisting of: a transplantation disorder, an autoimmune disease, cancer, multiple sclerosis, graft versus host disease, Kawasaki syndrome, an immunodeficiency disorder and an inflammatory disorder.

49. The use according to claim 48, wherein said autoimmune disease is selected from the group consisting of: rheumatoid arthritis, systemic lupus erythematosus, psoriasis, Sjogren's Syndrome, thyroiditis, Graves' disease, pulmonary fibrosis, bronchiolitis obliterans, hemolytic anemia and Wegener's granulomatosis.

50. The use according to claim 48, wherein said cancer is leukemia or lymphoma.

51. The use according to claim 48, wherein said immundeficiency disorder is AIDS.

52. The use according to claim 48, wherein said inflammatory disorder is selected from the group consisting of: asthma, allergies, adult respiratory distress syndrome and acute pancreatitis and chronic pancreatitis.

53. The use of a composition of claims 21 or 22 for the treatment of a disease condition that relates to T cells.


SEQUENCE LISTING (34 sequences)

SEQ ID NO: 1 - DNA, 973 bp, Homo sapiens
agcggccgcttttttattcccgggcatggtgctgggcctcctggtgcagatctgggccctgcaagaagcctcaagcctgagcgtgcagcaggggcccaacttgccgcaggtgaggcagggcagtcaggcgaccctggtctgccaggtggaccaggccacagcctgggaacggctccgtgttaagtggacaaaggctggggccatcctgtgtcaaccgtacatcaccaacggcagcctcagcctgggggtctgcgggccccagggacggctctcctggcaggcacccagccatctcaccctgcagctggaccctatgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgttgggtgcctggttctggggccgccgcagctgccagcaaagggactcaggtaacagcccaggtaagggagggtagggcggaggaggtgggaactgcagctgcttgacaaagacccactgcatctgtgctcccgcctgggggaaggatggaggagggatgaagggaggagaagcctggctcctatatcctatgctcatgtttttcagaaatagagacaaagcccctagcctgtgcaacctagcaagatcccatctctacaaaaaaattacaaattgctgggcgcggtggctcatgcccgtcatcccagcattttgggaggccgaggtgggcgaatcacctgaggtcaacctggacaatatggtgaaaccttgtatctactaaaaatacaaaaaattagctaggtgtggtggcagacgcctgtaataccagctactttgggaggctgaggcaggaggatcgctt

SEQ ID NO: 2 - protein, 186 aa, Homo sapiens
MVLGLLVQIWALQEASSLSVQQGPNLPQVRQGSQATLVCQVDQATAWERLRVKWTKAGAILCQPYITNGSLSLGVCGPQGRLSWQAPSHLTLQLDPMSLNHSGAYVCWAAVEIPELEEAEGNITRLFVDPDDPTQNRNRIASFPGFLFVLLGVGSMGVAAIVLGAWFWGRRSCQQRDSGNSPGKGG

SEQ ID NO: 3 - DNA, 232 bp, Homo sapiens
acatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaag

SEQ ID NO: 4 - DNA, 450 bp, Homo sapiens
ccatcctaatacgactcactatagggctcgagcggccgcccgggcaggtctagaattcagcggccgcttttttattcccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaaca

SEQ ID NO: 5 - DNA, 550 bp, Homo sapiens
ccacacagaacagaaaccggatcgcaagcttcccaggattcctcttcgtgctgctgggggtgggaagcatgggtgtggctgcgatcgtgttgggtgcctggttctggggccgccgcagctgccagcaaagggactcaggtaacagcccaggtaagggagggtagggcggaggaggtgggaactgcagctgcttgacaaagacccactgcatctgtgctcccgcctgggggaaggatggaggagggatgaagggaggagaagcctggctcctatatcctatgctcatgtttttcagaaatagagacaaagcccctagcctgtgcaacctagcaagatcccatctctacaaaaaattacaaattgctgggcgcggtggctcatgcccgtcatcccagcattttgggaggccgaggtgggcgaatcacctgaggtcaacctggacaatatggtgaaaccttgtatctactaaaaatacaaaaaattagctaggtgtggtggcagacgcctgtaataccagctactttgggaggctgaggcaggaggatcgctt

SEQ ID NO: 6 - protein, 139 aa, Mus sp.
MERNLVAVLGILWVQICWVSGDQVKQSPSALSLQEGTSSALRCNFSIATTTVQWFLQNSRGSLMNLFYLVPGTKENGRLKSTFNSKESYSTLHIRDAQLEDSGTYFCAAEVEGTGSKLSFGKGAKLTVSPDIQNPEPAV

SEQ ID NO: 7 - DNA, 21 bp, Artificial Sequence
  Description of Artificial Sequence Primer
acatcaccaacggcagcctca

SEQ ID NO: 8 - DNA, 21 bp, Artificial Sequence
  Description of Artificial Sequence Primer
cttgcgatccggtttctgttc

SEQ ID NO: 9 - DNA, 26 bp, Artificial Sequence
  Description of Artificial Sequence Primer
tgttgccctcagcctcctccaactca

SEQ ID NO: 10 - DNA, 27 bp, Artificial Sequence
  Description of Artificial Sequence Primer
accccacacagaacagaaaccggatcg

SEQ ID NO: 11 - DNA, 27 bp, Artificial Sequence
  Description of Artificial Sequence Primer
ccatcctaatacgactcactatagggc

SEQ ID NO: 12 - protein, 9 aa, Artificial Sequence
  Description of Artificial Sequence Synthetic Substrate Peptide
APRTPGGRR

SEQ ID NO: 13 - DNA, 10500 bp, Homo sapiens
tggggaaactgaggcacgaggtggcaggaagtggagcccccagcaggctggaacaccggacgtctgttgtctggccctgggtgttgcatctgcttctgcagcgggaggcggagattgtggcaggaagtctgtcaactgggagggggagaggggggtgatgggccaggaatggggtccccgggcatggtgctgggcctcctggtgcagatctggggtgagtatctgggcctccctcccctctgaaccccactcttgctgtcatccccagctgcctgccccccagctcagggcccctctaaacttcagtctctctactgtcagataaggcccatctccctccgagactcagaacgtgatgggggctggaggattaaggaaggatcctctgagggcccctaggatacaatctaaaccttgccctacataccagcctggcctggcctctctcatctccaccctccgcagtgcctttgctccctccctcttgctctagcctggggacttttctttctctgtttccgcaatgggaaggctggatctggccttcgagcctttgcacctgctgttccagctgcctaatacccttctccagatctcatgtctctaccctcctctgcaattgcatctcaaatgccaggaacttcacataacctcaacttctccgggggtctaacagacacgtcagacttctcacagctaaaagcagactttctactccggcagcagctccctctgtgtttccaattaaggacaaaggctgctgcccttcacacatttcctggggcgaaaacctccattcctctttccctctcctctgattccagccgcttgcacttttttgttgttcatttgtgtttttttgtttgtttgttttgttttgttttgagaaggagtcttgctctgtcacccaggctagagtgcagtgacgcgatctcggttgactgcaacctctgcctcccaggttcaagagattctcctgcctcaggctcccgagtagctgggactgcaggcgcgcgccactgcgcctggctaattttttttgtatttttagtagagacggggtttcgccatctcggccaggtgggtctcgaactcctgacctcatgatccacccaccttggcctcccaaagtgctgggattacaggcgtgagccaccgtgcccagtcatttttgttttttagagatgggtctcactatgttgcccaggctggtcttgaactcctggcctcaagcaatcacccagtcttggccacccaaagggctgggataacaggcatgagccaccacacccaaccaaggtctttttattaaaacttaaattggagactgaggcaggtggatcacttgaggtcaggagttccagaccagcctggccatgttggtgaaaccccatctctaccaaaaatacaaaaattagctgggcgtggtagcgagtgcctgtagtcccagctactcaagaggctgagccacaagaatcacttgaacctgggaggcagaggttgcaatgagccgagatcacaccactgcactcccacctgggcaacagagtgagaccccatctcaaaacaaaaacaaaaacaaaaacaaaaacttcaattggactatttcttccctgctttctgattttccatcacacttacattaaaacataaactgcttaacttccttcacctccccacaccaccccaaggctcacaggccctgcccaacccttcctccctctctccctgtcctcactctgctccagccacacgggcctcctcgctgttcctccaacacgccaggcacagtcctgccccagggcctttgcatgggctgtgcactgcctggaatgctcttaccccccaataccttccttctcacactggttatacctttccattaagatccctggtcaggctgagcacagtggcatgtacttgtaacaccagtaatttgggaggctgaggtgggaggatcacatgagcccaggaatgttttgttttgttttttattttgagacacagtctcgctctgtcgccaggctggagtgcagtggcgcgatctcagctcactgcaacctccgcctcccgggttcaagtgattctcccgcctcagcctcccgagtagctgggactacgggtacatgccaccacacccagctaatttttgtatttttagtagagacggggtttcaccatgttgggcaggatggtcttgatctcctgacctcgtgatccgcccgcctctgcctcccaaagtgctgggattataggcgtgagccactgcacccggccttgttttgttttgtttgagacagagcctcgctctgtcacccaggctggagtgcagtggcacgatctcaggtcactgcaacctccacctcccgggttcaagcgattctcctgcctcagcctcccaagtagctgggattacaggcacccgccaccatgcccagctaatttttgtagtttaagtagagacagggtttcaccatgttggccaggctggtcttgaactcctgacctcaggtgatccacccgcctcggcctcccaaagtgctgggattacaggcgtgagccactgcacccggcccatgaacccaggaggttgatcctgcagtgagctatgaaggtgccactgcactccagactgggcaatagaatgagatggagtctttttttctttgctatggagtctcgctctgttgcccaagctagagtgcagtggcagggtctcagctcactgcaacctctgcctctcggattcaagtgattctcctgcctcagcctcccatgtagctgggattccaggagcccgccaccacactggctaatttttgtatttttagtagagatggagtttcaccacgttggccagacgggtctcgaactcctgacctcaggtgatccaccggccttgacttcccaaagtacagggattacaggcatgagtcacagcacccagcccatgagcccaagcggctgaggctgcaatgaactttgatggtgccactgcgctccagactgggcaacagagtgagatggagtctttttttcttatatatggagtctcactctgttgcccaggctagagtgcagtggcgtggtcttaagaaaaaaaaaaaggccggtcacagtggctcacatctacaatcccagcactttgggaggcgttagcggggaggatcacttgagcccaggaattcaacaccagcctgggcaacacagtgagaccccaatctctacaaaaccaaaaaaattagccaggcatagtggttacatgcctgtagtcccagctacttgggaggctgaggtaggaggattactttagcccaggagttcagggctgccgtgagctgtgatggtgccactgcactccagcctggactatagagccagaccccaactcaaaaaaaattaaaaaaataaaaaatcaccacctcaaggaggccttccctgattactctcaaggaatagatgctccctgacctcactggtgtctgctgaggccactgccattcctttcccccactgcagttctcatttcacgacttcctgtgtctctcttgccctagattgtcagcttctgaagggcagggattttcatctgttttggtcatgcctgtgtctgtgttccccacacccagcatagggcctggcatatactaggcggtatttgtgtagctgagtactggagactatctcagaattccacaagctctgagcgacttatacccatcttacagatggggttgttgagcctcagcaagacatgaccttccaaaggtcatacagacagtgaggttctttggttttttttttatgttaaatttttagagacagggtgttgctttgtcacccaggctggagtgcagtggtgcaatcatagctcaccgcagcctcgaacgcctgggctcaagcgatcccttcacttagtctcttaaatagctgggcccacggtgcacgccaccatacccagctactttttaaatatttattgtagagatgaggtcttgctatgttcccaggctggtctggaactcctgggctcaagcaatcctcccacctcgggcttccaaagtgctgggactacaggcgtgagccactgcacccgtcttagacaagtgaggtttcaaacccagactgcgggctcactaggggagtgagtcacacagcctcacatgcgcacagtcagaaaggtcgcctctacctcttctgtcctccagccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccaggtacgggagccagcggggagagggaggggcagtggggaggatcctagaatcttaagacttttgagtcttaaactcctagaaacaaaaaactctaaattcttcatatcaaacttttttttttttttttaagagacagagtcttgctctgtcgtccaggccttggagtgcaggggcgcgatctcggctcactgcaacctccgcctcctaggttcaagcgattctcccgcctcagcctcccaagtagctgggattacaggcacgtgccagcacgcccagctaatttttgtatttttagtagagacgaggtttcaccatgttggccaggatagtctcgatctcttgacctcgtgatctgcctgcctcagcctcccaaagtactgggattacaggcctgagccaccatgcccggcccataatccaagaattttaggatacaaaattgaaagtcatcagccaggcatagtggctgatgcttgtaatcccagcattttgggaggctaaggcaagaggatcacctgagcccaggagctggaggctgcagtgagctatgagtgcaccactgcactccagcctgggcgacagagcgagaccctgtctctaaataaaataaagccatcagctgggcgcggtagctcatccctgtaatcctagcactttggaaggccaaggtgggtggatcacctgaggtcaggggttcgagaccagcctggccaacatgatgaaaaccccctctctactaaaaatacaaaaagtagctgggcatggtggtgtgagcctgtaattccagctactcaggaggctgaggcaggagaatccgggagggggaggttgcagtgagctcagattgtaccactgcactccagcctgggctacagagcaagactctgtctcaaaaaaaaaaaaaaaaaaaagccacagactctcaaagttggaagaaaccctccagatagctttccaggtcagcaacgccagctgcctcactttacagatgagccaggtgaaccagagagggccaagacatatctagcgttatgcaccaggcaggggtgcaccctatgcactgtcccctttgccatgtcccccacttatggacccatcctgggcagaatcgggaaacccaaggggatgcaggctttgcctcctgctctgtgagaggtgcctgtgggccccgggcctctgtccagccccctctggcagaatgaagcacccagctctcagcccctagagatgcctgttcctctccagctggtaggaccagcctgctggcgggggacacgtgcctgcctcaagggaggggtgtgttatggctgtgttacccacctcttcccacccctctgatgtcaaaggggccaattagcaattagaggaggcggggtggtggaggcgccaggctgtgcgttggcagctgtgagagagcagggagtgggcgcgggtccaaccccacacagctggcttccccacaccccaccaatcctgaacagcagcttggcagtccccagggatcccctgccaacctcagaggctgcaaaagaagggggttggggtgctggaccttatcttaattgcggcattcacagcctttagctggggttcctgtaaattatacaacctggattcaatcctctgtttcatttacttgctgtgtgaccttgagcaagtgacttgacctctctgtgccagttccctctgctgtcaaaatacagtgaatagtttttcctcatcaggtcgggtgactttagagcagttatagccaaagtgcttgctataatccaagtgcagtggctcacgcctgatatcccagcactttgggaggccaaggtgggaggattgtttgaggccaggaggtcgagaccagcttggccaacacagcaagacctcatctctacaagaaattctaaaattagccaggcatggtggcatatacccatagtcccagctacttgagaggttgaggcgggaggatcacttgagcccaggagttggagactgcagtgagctacgatcacgccactgcactccagcctgggtgacacagagcgaccctgtctccagaaaaacaaacaacaacaacaaaacaatgcttagcacagggcctgagacatagtaagttctcaaggaacttcagtgttaggatctccgagctgaggtgagacagggctttgtccaaagctggttcctgctgccgcctgggtaggggaccctaggcaagtgacttaggctctctgagcctcggtttccccgtctataaaatgggcgtggcatggagtagaggctcaaacaaaaacaaaaacaaccaaacaggatctggagagtgatagatcccaggataaagatggcggggctcatgatgacaaatcccagttggcagtttttccacaatgacccaacaagacagggacgatgagcctcatttttctttttttttttcgagacagtctcgctctgtcgcccaggctggagtgcagtggctcgatcttggctcactgcaagctccgcctcccgggttcacaccattctcctgcctcagcctcccgagtagctgggactacaggtgcccaccaccatggccggctaattttttgtatttttagtagagacggggtttcactgtgttagccaggatggtctcgatcgcctgaccttgtgatccgcctacctcggtctcccaaagtgctaagattacaggtgtgagccactgcgcctggccttctttttttttttttttgaggcagagtcttgctctgtcacccaggctggagcgtagtgacacgatctcggctcactgcaacctccccctcctggattcaagcgattctcctgcctcagcctcctgagtagttgggattaaaggcgccaccaccatgcctggctaatttttatatttttttgtagagatggggtttcaccatgttggccaggctggtctcgaactcctgacctcgagtgatcctcctgcctcagcctcccagagtgctcggattacaggcctgagccaccgcgtcctgctgagcctcatttttcagaagaagaaacagaggttcagagtgaacacttgcccaaatccacaaagcctggcaggaggaggaacacttaggtccccccaagtgagctctgtccttggagggtggagaagcctggcacccccgtgacatagatttcttctctgcagatgaccccacacagaacagaaaccggatcgcaagcttcccaggtgagccctgccctgtgttcctcccccagcgtcttccacccgcatcaccaccccttcccctcctgattagaccatctcttctccccctacccaggattcctcttcgtgctgctgggggtgggaagcatgggtgtggctgcgatcgtgtggggtgcctggttctggggccgccgcagctgccagcaaagggactcaggtaacagcccaggtaagggagggtagggcggaggaggtgggaactgcagctgcttgacaaagacccactgcatctgtgctcccgcctgggggaaggatggaggagggatgaagggaggagaagcctggctcctatatcctatgctcatgtttttcagaaatagagacaaagcccctagcctgtgcaacctagcaagatcccatctctacaaaaaaattacaaattgctgggcgcggtggctcatgcctgtcatcccagcattttgggaggccgaggtgggcgaatcacctgaggtcagcctggacaatatggtgaaaccttgtatctactaaaaatacaaaaaattagctaggtgtggtggcagacgcctgtaataccagctacttgggaggctgaggcaggaggatcgcttgaacccgggaggcagaggttgcagtgagccaagatcgcgccactgaactctagcctgggtgacagagcaagacgcggtctcaaaaaaaaaaaaatttaaaaattaggctgtgcatggtgtctcatgcctgtcatcccagcactttgggaggctgaggcgggtggatcacttgaggtcaggagtttgagaccagcctgggcgacatggtgaaacccagtctctactaaaaatacaaacattacccaggcgtggtggcctgtgcctgtagtcccagctgcttgggaggttgaggcaggagaatcactggaacccaggaggcagagattgcagtgagcccagactgcctcactacactccagcctgggcgacacagccagactccatctcaacaacaacaaaaaaattagcctggcttggtggcacacgcctgtggtcccagctacttgggaggctgaggtgtgaggattgcttgaacctgggaggcggaggctgcagtgagctatagtttccccactgtactccagcctgggtgacagagagagaccctgtctcaaaaaacaaacaaacaaacaaaaagacagtttgaaattaaaaaaaaaaaaaaattggccgggtgcagtggctcatgcatgtaatcccagcattttgggaggccacggcgggcgaatcacgaggccaggagttcgagaccagcctggccaacgtggtgaaaccccatctctgctaaaaatacaaaaaattagccagacgtagtagcgggggcttgtaatcccagctactagggagactgaggcaggagaatcacttgaacctgggaggtggagactgcagtgagctgagatcgcaccgctgctacactccagcccaggtgacagagtaagactgtctcaaaaaaaaaaaaaaaaaggccgggcgcggtggctcacgcctgtaatcccagcactttgggaggccgaggcgggtggatcacgaggtcaggagatcaagaccatcctggctaacagtgaaaccccgtctctactaaaaatacaaaaaatcagccaggcatggtggcgggagcctgtagtcccagctagttgggaggctgaggcaggagaatggcgtgaacccaggaggcgaagcttgcagtgagccgagattgcgccactgcactccagcctgggcgacagagtgagactccgtctcacagaaaaaaaaaaaaagagacagatcccccagaggtcagctggagagggcaggactctcttaagtgatcctcttggtctgtgtcatccatcctaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtgagagatcccaggagacctcaacaggaccccacccataggtacacacaaaaaaggggggatcgaggccagacacggtggctcacgcctgtaatcccagcagtttgggaagccgaggcgggtggaacacttgaggtcaggggtttgagaccagcctggcttgaacctgggaggcggaggttgcagtgagccgagattgcgccactgcactccagcctgggcgacagagtgagactccgtctcaaaaaaaacaaaaagcaggaggattgggagcctgtcagccccatcctgagaccccgtcctcatttctgtaatgatggatctcgctcccactttcccccaagaacctaataaaggcttgtgaagaaaaagcaaagctggtgtttgtggcgatttgggagtgctaaaaagatctgaagaaatcgggtgcggtggctcacgcctctaatcccagcactttgggaggctgcggtggaaggatcacttgaagccaagagtttgagaccagcctgggcaatagagcaagaagactccatttctacagaaaat

SEQ ID NO: 14 - DNA, 1065 bp, Homo sapiens
  CDS (10)..(843)
gggccaggaatggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtgagagatcccaggagacctcaacaggaccccacccataggtacacacaaaaaaggggggatcgaggccagacacggtggctcacgcctgtaatcccagcagtttgggaagccgaggcgggtggaacacttgaggtcaggggtttgagaccagcctggcttgaacctgggaggcggaggttgcagtgagccgagattgcgccactgcactccagcctgggcg

SEQ ID NO: 15 - protein, 278 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 16 - DNA, 417 bp, Homo sapiens
atggtgctgggcctcctggtgcagatctgggccctgcaagaagcctcaagcctgagcgtgcagcaggggcccaacttgccgcaggtgaggcagggcagtcaggcgaccctggtctgccaggtggaccaggccacagcctgggaacggctccgtgttaagtggacaaaggctggggccatcctgtgtcaaccgtacatcaccaacggcagcctcagcctgggggtctgcgggccccagggacggctctcctggcaggcacccagccatctcaccctgcagctggaccctatgagcctcaaccacagcggggcgtacgtgtgctgggcggccgtagagattcctgagttggaggaggctgagggcaacataacaaggctctttgtggacccagatgaccccacacagaacagaaaccgg

SEQ ID NO: 17 - protein, 139 aa, Homo sapiens
MVLGLLVQIWALQEASSLSVQQGPNLPQVRQGSQATLVCQVDQATAWERLRVKWTKAGAILCQPYITNGSLSLGVCGPQGRLSWQAPSHLTLQLDPMSLNHSGAYVCWAAVEIPELEEAEGNITRLFVDPDDPTQNRNR

SEQ ID NO: 18 - DNA, 417 bp, Homo sapiens
atggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccgg

SEQ ID NO: 19 - protein, 124 aa, Homo sapiens
MGSPGMVLGLLVQIWALQEASSLSVQQGPNLLQVRQGSQATLVCQVDQATAWERLRVLGVCGPQGRLSWQAPSHLTLQLDPVSLNHSGAYVCWAAVEIPELEEAEGNITRLFVDPDDPTQNRNR

SEQ ID NO: 20 - DNA, 237 bp, Homo sapiens
ggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggcc

SEQ ID NO: 21 - protein, 59 aa, Homo sapiens
GSQATLVCQVDQATAWERLRVLGVCGPQGRLSWQAPSHLTLQLDPVSLNHSGAYVCWAA

SEQ ID NO: 22 - DNA, 81 bp, Homo sapiens
atcgtgttgggtgcctggttctggggccgccgcagctgccagcaaagggactcaggtaacagcccaggtaagggagggtag

SEQ ID NO: 23 - protein, 26 aa, Homo sapiens
IVLGAWFWGRRSCQQRDSGNSPGKGG

SEQ ID NO: 24 - DNA, 342 bp, Homo sapiens
atcgtgtggggtgcctggttctggggccgccgcagctgccagcaaagggactcaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 25 - protein, 113 aa, Homo sapiens
IVWGAWFWGRRSCQQRDSGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 26 - DNA, 324 bp, Homo sapiens
ggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccgg

SEQ ID NO: 27 - protein, 88 aa, Homo sapiens
GSQATLVCQVDQATAWERLRVLGVCGPQGRLSWQAPSHLTLQLDPVSLNHSGAYVCWAAVEIPELEEAEGNITRLFVDPDDPTQNRNR

SEQ ID NO: 28 - DNA, 468 bp, Homo sapiens
ggcagtcaggcgaccctggtctgccaggtggaccaggccacagcctgggaacggctccgtgttaagtggacaaaggctggggccatcctgtgtcaaccgtacatcaccaacggcagcctcagcctgggggtctgcgggccccagggacggctctcctggcaggcacccagccatctcaccctgcagctggaccctatgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgttgggtgcctggttctggggccgccgcagctgccagcaaagggactcaggtaacagcccaggtaagggagggtag

SEQ ID NO: 29 - protein, 155 aa, Homo sapiens
GSQATLVCQVDQATAWERLRVKWTKAGAILCQPYITNGSLSLGVCGPQGRLSWQAPSHLTLQLDPMSLNHSGAYVCWAAVEIPELEEAEGNITRLFVDPDDPTQNRNRIASFPGFLFVLLGVGSMGVAAIVLGAWFWGRRSCQQRDSGNSPGKGG

SEQ ID NO: 30 - DNA, 729 bp, Homo sapiens
ggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtga

SEQ ID NO: 31 - protein, 242 aa, Homo sapiens
GSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 32 - DNA, 63 bp, Homo sapiens
atcgcaagcttcccaggattcctcttcgtgctgctgggggtgggaagcatgggtgtggctgcg

SEQ ID NO: 33 - protein, 21 aa, Homo sapiens
IASFPGFLFVLLGVGSMGVAA

SEQ ID NO: 34 - DNA, 550 bp, Unknown Organism
  Description of Unknown Organism EST Assembly with template identification number 230543.1
gtgggggtgatgggccagtgaatggggtccccgggcatggtgctgggcctactggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgca